SEQUENCE LISTING (260 sequences)

SEQ ID NO: 1 - DNA, 2274 bp, Homo sapiens
atgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtga

SEQ ID NO: 2 - protein, 757 aa, Homo sapiens
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 3 - DNA, 2298 bp, Mus musculus
atgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgtga

SEQ ID NO: 4 - protein, 765 aa, Mus musculus
MLLRLLLAWVAAVPALGQVPWTPEPRAACGPSSCYALFPRRRTFLEAWRACRELGGNLATPRTPEEAQRVDSLVGVGPANGLLWIGLQRQARQCQPQRPLRGFIWTTGDQDTAFTNWAQPATEGPCPAQRCAALEASGEHRWLEGSCTLAVDGYLCQFGFEGACPALPLEVGQAGPAVYTTPFNLVSSEFEWLPFGSVAAVQCQAGRGASLLCVKQPSGGVGWSQTGPLCPGTGCGPDNGGCEHECVEEVDGAVSCRCSEGFRLAADGHSCEDPCAQAPCEQQCEPGGPQGYSCHCRLGFRPAEDDPHRCVDTDECQIAGVCQQMCVNYVGGFECYCSEGHELEADGISCSPAGAMGAQASQDLRDELLDDGEEGEDEEEPWEDFDGTWTEEQGILWLAPTHPPDFGLPYRPNFPQDGEPQRLHLEPTWPPPLSAPRGPYHSSVVSATRPMVISATRPTLPSAHKTSVISATRPPLSPVHPPAMAPATPPAVFSEHQIPKIKANYPDLPFGHKPGITSATHPARSPPYQPPIISTNYPQVFPPHQAPMSPDTHTITYLPPVPPHLDPGDTTSKAHQHPLLPDAPGIRTQAPQLSVSALQPPLPTNSRSSVHETPVPAANQPPAFPSSPLPPQRPTNQTSSISPTHSYSRAPLVPREGVPSPKSVPQLPSVPSTAAPTALAESGLAGQSQRDDRWLLVALLVPTCVFLVVLLALGIVYCTRCGSHAPNKRITDCYRWVTHAGNKSSTEPMPPRGSLTGVQTCRTSV

SEQ ID NO: 5 - DNA, 2361 bp, Artificial Sequence
  Description of Artificial Sequence Synthetic polynucleotide
atgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtctagagggcccttcgaaggtaagcctatccctaaccctctcctcggtctcgattctacgcgtaccggtcatcatcaccatcaccattga

SEQ ID NO: 6 - DNA, 363 bp, Artificial Sequence
  Description of Artificial Sequence Synthetic polynucleotide
gcccagtcggtggaggagtccgggggagacctggtcaggccgggggcgtccctgacactcacctgctcagcttctggattcaccatcaataggaactactggatatgctgggtccgccaggctccagggagggggccggagtggatcgcttgtatttatggtggtagtagtggtaccacttactacacgaactgggcgaaaggccggttcaccatctccaaagccgcgtcgaccacggtgactctgcaaatgaccagtctgacagtcgcggacacggccacctatttctgtgcgagagttgataatggtggtgattggtatttcaggttgtggggcccaggcaccctggtcaccatctcctca

SEQ ID NO: 7 - DNA, 66 bp, Artificial Sequence
  Description of Artificial Sequence Synthetic oligonucleotide
gcccagtcggtggaggagtccgggggagacctggtcaggccgggggcgtccctgacactcacctgc

SEQ ID NO: 8 - DNA, 33 bp, Artificial Sequence
  Description of Artificial Sequence Synthetic oligonucleotide
tcagcttctggattcaccatcaataggaactac

SEQ ID NO: 9 - DNA, 51 bp, Artificial Sequence
  Description of Artificial Sequence Synthetic oligonucleotide
tggatatgctgggtccgccaggctccagggagggggccggagtggatcgct

SEQ ID NO: 10 - DNA, 54 bp, Artificial Sequence
  Description of Artificial Sequence Synthetic oligonucleotide
tgtatttatggtggtagtagtggtaccacttactacacgaactgggcgaaaggc

SEQ ID NO: 11 - DNA, 93 bp, Artificial Sequence
  Description of Artificial Sequence Synthetic oligonucleotide
cggttcaccatctccaaagccgcgtcgaccacggtgactctgcaaatgaccagtctgacagtcgcggacacggccacctatttctgtgcgaga

SEQ ID NO: 12 - DNA, 33 bp, Artificial Sequence
  Description of Artificial Sequence Synthetic oligonucleotide
gttgataatggtggtgattggtatttcaggttg

SEQ ID NO: 13 - DNA, 33 bp, Artificial Sequence
  Description of Artificial Sequence Synthetic oligonucleotide
tggggcccaggcaccctggtcaccatctcctca

SEQ ID NO: 14 - protein, 121 aa, Artificial Sequence
  Description of Artificial Sequence Synthetic polypeptide
AQSVEESGGDLVRPGASLTLTCSASGFTINRNYWICWVRQAPGRGPEWIACIYGGSSGTTYYTNWAKGRFTISKAASTTVTLQMTSLTVADTATYFCARVDNGGDWYFRLWGPGTLVTISS

SEQ ID NO: 15 - protein, 22 aa, Artificial Sequence
  Description of Artificial Sequence Synthetic peptide
AQSVEESGGDLVRPGASLTLTC

SEQ ID NO: 16 - protein, 11 aa, Artificial Sequence
  Description of Artificial Sequence Synthetic peptide
SASGFTINRNY

SEQ ID NO: 17 - protein, 17 aa, Artificial Sequence
  Description of Artificial Sequence Synthetic peptide
WICWVRQAPGRGPEWIA

SEQ ID NO: 18 - protein, 18 aa, Artificial Sequence
  Description of Artificial Sequence Synthetic peptide
CIYGGSSGTTYYTNWAKG

SEQ ID NO: 19 - protein, 31 aa, Artificial Sequence
  Description of Artificial Sequence Synthetic polypeptide
RFTISKAASTTVTLQMTSLTVADTATYFCAR

SEQ ID NO: 20 - protein, 11 aa, Artificial Sequence
  Description of Artificial Sequence Synthetic peptide
VDNGGDWYFRL

SEQ ID NO: 21 - protein, 11 aa, Artificial Sequence
  Description of Artificial Sequence Synthetic peptide
WGPGTLVTISS

SEQ ID NO: 22 - DNA, 330 bp, Artificial Sequence
  Description of Artificial Sequence Synthetic polynucleotide
gagctcgatctgacccagactccagcctctgtggaggtagctgtgggaggcacagtcaccatcaagtgccaggccagtcagagcattagtagctacttagcctggtatcagcagaaaccaggacagcctcccaagctcctgatctacagggcatccacgctggcatctggggtctcatcgcggttcaaaggcagtggatctgggacagagttcactctcaccatcagcggtgtgcagtgtgacgatgctgccacttactactgtcaacagggtgtgcgtgtggttgatattgataattctttcggcggagggaccgaactggtggtcaaa

SEQ ID NO: 23 - DNA, 69 bp, Artificial Sequence
  Description of Artificial Sequence Synthetic oligonucleotide
gagctcgatctgacccagactccagcctctgtggaggtagctgtgggaggcacagtcaccatcaagtgc

SEQ ID NO: 24 - DNA, 33 bp, Artificial Sequence
  Description of Artificial Sequence Synthetic oligonucleotide
caggccagtcagagcattagtagctacttagcc

SEQ ID NO: 25 - DNA, 45 bp, Artificial Sequence
  Description of Artificial Sequence Synthetic oligonucleotide
tggtatcagcagaaaccaggacagcctcccaagctcctgatctac

SEQ ID NO: 26 - DNA, 21 bp, Artificial Sequence
  Description of Artificial Sequence Synthetic oligonucleotide
agggcatccacgctggcatct

SEQ ID NO: 27 - DNA, 96 bp, Artificial Sequence
  Description of Artificial Sequence Synthetic oligonucleotide
ggggtctcatcgcggttcaaaggcagtggatctgggacagagttcactctcaccatcagcggtgtgcagtgtgacgatgctgccacttactactgt

SEQ ID NO: 28 - DNA, 36 bp, Artificial Sequence
  Description of Artificial Sequence Synthetic oligonucleotide
caacagggtgtgcgtgtggttgatattgataattct

SEQ ID NO: 29 - DNA, 30 bp, Artificial Sequence
  Description of Artificial Sequence Synthetic oligonucleotide
ttcggcggagggaccgaactggtggtcaaa

SEQ ID NO: 30 - protein, 110 aa, Artificial Sequence
  Description of Artificial Sequence Synthetic polypeptide
ELDLTQTPASVEVAVGGTVTIKCQASQSISSYLAWYQQKPGQPPKLLIYRASTLASGVSSRFKGSGSGTEFTLTISGVQCDDAATYYCQQGVRVVDIDNSFGGGTELVVK

SEQ ID NO: 31 - protein, 23 aa, Artificial Sequence
  Description of Artificial Sequence Synthetic peptide
ELDLTQTPASVEVAVGGTVTIKC

SEQ ID NO: 32 - protein, 11 aa, Artificial Sequence
  Description of Artificial Sequence Synthetic peptide
QASQSISSYLA

SEQ ID NO: 33 - protein, 15 aa, Artificial Sequence
  Description of Artificial Sequence Synthetic peptide
WYQQKPGQPPKLLIY

SEQ ID NO: 34 - protein, 7 aa, Artificial Sequence
  Description of Artificial Sequence Synthetic peptide
RASTLAS

SEQ ID NO: 35 - protein, 32 aa, Artificial Sequence
  Description of Artificial Sequence Synthetic polypeptide
GVSSRFKGSGSGTEFTLTISGVQCDDAATYYC

SEQ ID NO: 36 - protein, 12 aa, Artificial Sequence
  Description of Artificial Sequence Synthetic peptide
QQGVRVVDIDNS

SEQ ID NO: 37 - protein, 10 aa, Artificial Sequence
  Description of Artificial Sequence Synthetic peptide
FGGGTELVVK

SEQ ID NO: 38 - DNA, 363 bp, Artificial Sequence
  Description of Artificial Sequence Synthetic polynucleotide
gcccagtcgttggaggagtccgggggagacctggtcaagcctgagggatccctgacactcacctgcacagcctctggattctccttcagtagcagctactggggatgctgggtccgccaggctccagggaaggggcctgagtggatcgcatgcatttatggtggtagtagtggtaccacttattacccgaactgggcgaaaggccgattctccatctccaaaacctcgtcgaccacggtgactctgcaaatggccagtctgacagccgcggacacggccacctatttctgtgcgagagtgactaatggtggtgattgggattttaaattgtggggcccaggcaccctggtcaccatctcttca

SEQ ID NO: 39 - DNA, 75 bp, Artificial Sequence
  Description of Artificial Sequence Synthetic oligonucleotide
gcccagtcgttggaggagtccgggggagacctggtcaagcctgagggatccctgacactcacctgcacagcctct

SEQ ID NO: 40 - DNA, 33 bp, Artificial Sequence
  Description of Artificial Sequence Synthetic oligonucleotide
ggattctccttcagtagcagctactggggatgc

SEQ ID NO: 41 - DNA, 42 bp, Artificial Sequence
  Description of Artificial Sequence Synthetic oligonucleotide
tgggtccgccaggctccagggaaggggcctgagtggatcgca

SEQ ID NO: 42 - DNA, 54 bp, Artificial Sequence
  Description of Artificial Sequence Synthetic oligonucleotide
tgcatttatggtggtagtagtggtaccacttattacccgaactgggcgaaaggc

SEQ ID NO: 43 - DNA, 33 bp, Artificial Sequence
  Description of Artificial Sequence Synthetic oligonucleotide
cgattctccatctccaaaacctcgtcgaccacg

SEQ ID NO: 44 - DNA, 93 bp, Artificial Sequence
  Description of Artificial Sequence Synthetic oligonucleotide
gtgactctgcaaatggccagtctgacagccgcggacacggccacctatttctgtgcgagagtgactaatggtggtgattgggattttaaattg

SEQ ID NO: 45 - DNA, 33 bp, Artificial Sequence
  Description of Artificial Sequence Synthetic oligonucleotide
tggggcccaggcaccctggtcaccatctcttca

SEQ ID NO: 46 - protein, 121 aa, Artificial Sequence
  Description of Artificial Sequence Synthetic polypeptide
AQSLEESGGDLVKPEGSLTLTCTASGFSFSSSYWGCWVRQAPGKGPEWIACIYGGSSGTTYYPNWAKGRFSISKTSSTTVTLQMASLTAADTATYFCARVTNGGDWDFKLWGPGTLVTISS

SEQ ID NO: 47 - protein, 25 aa, Artificial Sequence
  Description of Artificial Sequence Synthetic peptide
AQSLEESGGDLVKPEGSLTLTCTAS

SEQ ID NO: 48 - protein, 11 aa, Artificial Sequence
  Description of Artificial Sequence Synthetic peptide
GFSFSSSYWGC

SEQ ID NO: 49 - protein, 14 aa, Artificial Sequence
  Description of Artificial Sequence Synthetic peptide
WVRQAPGKGPEWIA

SEQ ID NO: 50 - protein, 18 aa, Artificial Sequence
  Description of Artificial Sequence Synthetic peptide
CIYGGSSGTTYYPNWAKG

SEQ ID NO: 51 - protein, 11 aa, Artificial Sequence
  Description of Artificial Sequence Synthetic peptide
RFSISKTSSTT

SEQ ID NO: 52 - protein, 31 aa, Artificial Sequence
  Description of Artificial Sequence Synthetic polypeptide
VTLQMASLTAADTATYFCARVTNGGDWDFKL

SEQ ID NO: 53 - protein, 11 aa, Artificial Sequence
  Description of Artificial Sequence Synthetic peptide
WGPGTLVTISS

SEQ ID NO: 54 - DNA, 330 bp, Artificial Sequence
  Description of Artificial Sequence Synthetic polynucleotide
gagctcgtgctgacccagactccagcctctgtggaggtagctgtgggaggcacagtcaccatcaagtgccaggccagtcagaccattaataactacttgacctggtatcagcagaaaccaggacagcctcccaagctcctgatctacagggcatccactctggcatctggggtcccatcgcggttcaaaggcagtggatctgggacagagttcactctcaccatcagcgacctggagtgtgccgatgctgccacttactactgtcaacagactgttcgtgtggttgatgttgataatagtttcggcggagggaccgaggtggtggtcaaa

SEQ ID NO: 55 - DNA, 69 bp, Artificial Sequence
  Description of Artificial Sequence Synthetic oligonucleotide
gagctcgtgctgacccagactccagcctctgtggaggtagctgtgggaggcacagtcaccatcaagtgc

SEQ ID NO: 56 - DNA, 33 bp, Artificial Sequence
  Description of Artificial Sequence Synthetic oligonucleotide
caggccagtcagaccattaataactacttgacc

SEQ ID NO: 57 - DNA, 45 bp, Artificial Sequence; identical to SEQ ID NO: 25
  Description of Artificial Sequence Synthetic oligonucleotide

SEQ ID NO: 58 - DNA, 21 bp, Artificial Sequence
  Description of Artificial Sequence Synthetic oligonucleotide
agggcatccactctggcatct

SEQ ID NO: 59 - DNA, 96 bp, Artificial Sequence
  Description of Artificial Sequence Synthetic oligonucleotide
ggggtcccatcgcggttcaaaggcagtggatctgggacagagttcactctcaccatcagcgacctggagtgtgccgatgctgccacttactactgt

SEQ ID NO: 60 - DNA, 36 bp, Artificial Sequence
  Description of Artificial Sequence Synthetic oligonucleotide
caacagactgttcgtgtggttgatgttgataatagt

SEQ ID NO: 61 - DNA, 30 bp, Artificial Sequence
  Description of Artificial Sequence Synthetic oligonucleotide
ttcggcggagggaccgaggtggtggtcaaa

SEQ ID NO: 62 - protein, 110 aa, Artificial Sequence
  Description of Artificial Sequence Synthetic polypeptide
ELVLTQTPASVEVAVGGTVTIKCQASQTINNYLTWYQQKPGQPPKLLIYRASTLASGVPSRFKGSGSGTEFTLTISDLECADAATYYCQQTVRVVDVDNSFGGGTEVVVK

SEQ ID NO: 63 - protein, 23 aa, Artificial Sequence
  Description of Artificial Sequence Synthetic peptide
ELVLTQTPASVEVAVGGTVTIKC

SEQ ID NO: 64 - protein, 11 aa, Artificial Sequence
  Description of Artificial Sequence Synthetic peptide
QASQTINNYLT

SEQ ID NO: 65 - protein, 15 aa, Artificial Sequence
  Description of Artificial Sequence Synthetic peptide
WYQQKPGQPPKLLIY

SEQ ID NO: 66 - protein, 7 aa, Artificial Sequence
  Description of Artificial Sequence Synthetic peptide
RASTLAS

SEQ ID NO: 67 - protein, 32 aa, Artificial Sequence
  Description of Artificial Sequence Synthetic polypeptide
GVPSRFKGSGSGTEFTLTISDLECADAATYYC

SEQ ID NO: 68 - protein, 12 aa, Artificial Sequence
  Description of Artificial Sequence Synthetic peptide
QQTVRVVDVDNS

SEQ ID NO: 69 - protein, 10 aa, Artificial Sequence
  Description of Artificial Sequence Synthetic peptide
FGGGTEVVVK

SEQ ID NO: 70 - DNA, 363 bp, Artificial Sequence
  Description of Artificial Sequence Synthetic polynucleotide
cagcagcagctgatggagtccgggggagacctggtcaagcctgagggatccctgacactcacctgcacagcctctggattctccttcagtagcagctactggggatgctgggtccgccaggctccagggaaggggcctgagtggatcgcatgcatttatggtggtagtagtggtaccacttattacccgaactgggcgaaaggccgattctccatctccaaaacctcgtcgaccacggtgactctgcaaatggccagtctgacagccgcggacacggccacctatttctgtgcgagagtgactaatggtggtgattgggattttaaattgtggggcccaggcaccctggtcaccgtctcctca

SEQ ID NO: 71 - DNA, 75 bp, Artificial Sequence
  Description of Artificial Sequence Synthetic oligonucleotide
cagcagcagctgatggagtccgggggagacctggtcaagcctgagggatccctgacactcacctgcacagcctct

SEQ ID NO: 72 - DNA, 33 bp, Artificial Sequence; identical to SEQ ID NO: 40
  Description of Artificial Sequence Synthetic oligonucleotide

SEQ ID NO: 73 - DNA, 42 bp, Artificial Sequence; identical to SEQ ID NO: 41
  Description of Artificial Sequence Synthetic oligonucleotide

SEQ ID NO: 74 - DNA, 54 bp, Artificial Sequence; identical to SEQ ID NO: 42
  Description of Artificial Sequence Synthetic oligonucleotide

SEQ ID NO: 75 - DNA, 33 bp, Artificial Sequence; identical to SEQ ID NO: 43
  Description of Artificial Sequence Synthetic oligonucleotide

SEQ ID NO: 76 - DNA, 93 bp, Artificial Sequence; identical to SEQ ID NO: 44
  Description of Artificial Sequence Synthetic oligonucleotide

SEQ ID NO: 77 - DNA, 33 bp, Artificial Sequence
  Description of Artificial Sequence Synthetic oligonucleotide
tggggcccaggcaccctggtcaccgtctcctca

SEQ ID NO: 78 - protein, 121 aa, Artificial Sequence
  Description of Artificial Sequence Synthetic polypeptide
QQQLMESGGDLVKPEGSLTLTCTASGFSFSSSYWGCWVRQAPGKGPEWIACIYGGSSGTTYYPNWAKGRFSISKTSSTTVTLQMASLTAADTATYFCARVTNGGDWDFKLWGPGTLVTVSS

SEQ ID NO: 79 - protein, 25 aa, Artificial Sequence
  Description of Artificial Sequence Synthetic peptide
QQQLMESGGDLVKPEGSLTLTCTAS

SEQ ID NO: 80 - protein, 11 aa, Artificial Sequence
  Description of Artificial Sequence Synthetic peptide
GFSFSSSYWGC

SEQ ID NO: 81 - protein, 14 aa, Artificial Sequence
  Description of Artificial Sequence Synthetic peptide
WVRQAPGKGPEWIA

SEQ ID NO: 82 - protein, 18 aa, Artificial Sequence
  Description of Artificial Sequence Synthetic peptide
CIYGGSSGTTYYPNWAKG

SEQ ID NO: 83 - protein, 11 aa, Artificial Sequence
  Description of Artificial Sequence Synthetic peptide
RFSISKTSSTT

SEQ ID NO: 84 - protein, 31 aa, Artificial Sequence; identical to SEQ ID NO: 52
  Description of Artificial Sequence Synthetic polypeptide

SEQ ID NO: 85 - protein, 11 aa, Artificial Sequence
  Description of Artificial Sequence Synthetic peptide
WGPGTLVTVSS

SEQ ID NO: 86 - DNA, 331 bp, Artificial Sequence
  Description of Artificial Sequence Synthetic polynucleotide
gagctcgtgatgacccagactccagcctctgtggaggtagctgtgggaggcacagtcaccatcaagtgccaggccagtcagagcattaatacctacttagcctggtatcagcagaaaccagggcagcctcccaagctcctgatctacagggcatccactctggcatctggggtcccatcgcggttcaaaggcagtggatctgggacagagttcactctcaccattagcgacctggagtgtgccgatgctgccacttactactgtcaacagagtgttcgtgttattgatgttgataatactttcggcggagggaccgaggtggtcgtcaaaa

SEQ ID NO: 87 - DNA, 69 bp, Artificial Sequence
  Description of Artificial Sequence Synthetic oligonucleotide
gagctcgtgatgacccagactccagcctctgtggaggtagctgtgggaggcacagtcaccatcaagtgc

SEQ ID NO: 88 - DNA, 33 bp, Artificial Sequence
  Description of Artificial Sequence Synthetic oligonucleotide
caggccagtcagagcattaatacctacttagcc

SEQ ID NO: 89 - DNA, 45 bp, Artificial Sequence
  Description of Artificial Sequence Synthetic oligonucleotide
tggtatcagcagaaaccagggcagcctcccaagctcctgatctac

SEQ ID NO: 90 - DNA, 21 bp, Artificial Sequence
  Description of Artificial Sequence Synthetic oligonucleotide
agggcatccactctggcatct

SEQ ID NO: 91 - DNA, 96 bp, Artificial Sequence
  Description of Artificial Sequence Synthetic oligonucleotide
ggggtcccatcgcggttcaaaggcagtggatctgggacagagttcactctcaccattagcgacctggagtgtgccgatgctgccacttactactgt

SEQ ID NO: 92 - DNA, 36 bp, Artificial Sequence
  Description of Artificial Sequence Synthetic oligonucleotide
caacagagtgttcgtgttattgatgttgataatact

SEQ ID NO: 93 - DNA, 31 bp, Artificial Sequence
  Description of Artificial Sequence Synthetic oligonucleotide
ttcggcggagggaccgaggtggtcgtcaaaa

SEQ ID NO: 94 - protein, 110 aa, Artificial Sequence
  Description of Artificial Sequence Synthetic polypeptide
ELVMTQTPASVEVAVGGTVTIKCQASQSINTYLAWYQQKPGQPPKLLIYRASTLASGVPSRFKGSGSGTEFTLTISDLECADAATYYCQQSVRVIDVDNTFGGGTEVVVK

SEQ ID NO: 95 - protein, 23 aa, Artificial Sequence
  Description of Artificial Sequence Synthetic peptide
ELVMTQTPASVEVAVGGTVTIKC

SEQ ID NO: 96 - protein, 11 aa, Artificial Sequence
  Description of Artificial Sequence Synthetic peptide
QASQSINTYLA

SEQ ID NO: 97 - protein, 15 aa, Artificial Sequence
  Description of Artificial Sequence Synthetic peptide
WYQQKPGQPPKLLIY

SEQ ID NO: 98 - protein, 7 aa, Artificial Sequence
  Description of Artificial Sequence Synthetic peptide
RASTLAS

SEQ ID NO: 99 - protein, 32 aa, Artificial Sequence; identical to SEQ ID NO: 67
  Description of Artificial Sequence Synthetic polypeptide

SEQ ID NO: 100 - protein, 12 aa, Artificial Sequence
  Description of Artificial Sequence Synthetic peptide
QQSVRVIDVDNT

SEQ ID NO: 101 - protein, 10 aa, Artificial Sequence
  Description of Artificial Sequence Synthetic peptide
FGGGTEVVVK

SEQ ID NO: 102 - DNA, 378 bp, Artificial Sequence
  Description of Artificial Sequence Synthetic polynucleotide
caaccagccatggcccagtcggtggaggagtccgggggaggcctggtcaagcctgagggatccctgacactctcctgcaaagcctctggattctccttcagtaacaactcctacatatgctgggtccgccaggctccagggaagggtctggagtggatcgcatgcattgagtttggtcgtggtaactcacactacgcgagctgggcgaaaggccgattcaccatcaccagaagcaccagcctaaacacggtgactctgcaactgaacagtctgacagccgcggacacggccacctatttctgtgcgagaggatctcgtggtgttggttatggttatggcttgtggggcccaggcaccctggtcaccatctcttcaaag

SEQ ID NO: 103 - DNA, 87 bp, Artificial Sequence
  Description of Artificial Sequence Synthetic oligonucleotide
caaccagccatggcccagtcggtggaggagtccgggggaggcctggtcaagcctgagggatccctgacactctcctgcaaagcctct

SEQ ID NO: 104 - DNA, 33 bp, Artificial Sequence
  Description of Artificial Sequence Synthetic oligonucleotide
ggattctccttcagtaacaactcctacatatgc

SEQ ID NO: 105 - DNA, 42 bp, Artificial Sequence
  Description of Artificial Sequence Synthetic oligonucleotide
tgggtccgccaggctccagggaagggtctggagtggatcgca

SEQ ID NO: 106 - DNA, 51 bp, Artificial Sequence
  Description of Artificial Sequence Synthetic oligonucleotide
tgcattgagtttggtcgtggtaactcacactacgcgagctgggcgaaaggc

SEQ ID NO: 107 - DNA, 96 bp, Artificial Sequence
  Description of Artificial Sequence Synthetic oligonucleotide
cgattcaccatcaccagaagcaccagcctaaacacggtgactctgcaactgaacagtctgacagccgcggacacggccacctatttctgtgcgaga

SEQ ID NO: 108 - DNA, 33 bp, Artificial Sequence
  Description of Artificial Sequence Synthetic oligonucleotide
ggatctcgtggtgttggttatggttatggcttg

SEQ ID NO: 109 - DNA, 36 bp, Artificial Sequence
  Description of Artificial Sequence Synthetic oligonucleotide
tggggcccaggcaccctggtcaccatctcttcaaag

SEQ ID NO: 110 - protein, 121 aa, Artificial Sequence
  Description of Artificial Sequence Synthetic polypeptide
AQSVEESGGGLVKPEGSLTLSCKASGFSFSNNSYICWVRQAPGKGLEWIACIEFGRGNSHYASWAKGRFTITRSTSLNTVTLQLNSLTAADTATYFCARGSRGVGYGYGLWGPGTLVTISS

SEQ ID NO: 111 - protein, 25 aa, Artificial Sequence
  Description of Artificial Sequence Synthetic peptide
AQSVEESGGGLVKPEGSLTLSCKAS

SEQ ID NO: 112 - protein, 11 aa, Artificial Sequence
  Description of Artificial Sequence Synthetic peptide
GFSFSNNSYIC

SEQ ID NO: 113 - protein, 14 aa, Artificial Sequence
  Description of Artificial Sequence Synthetic peptide
WVRQAPGKGLEWIA

SEQ ID NO: 114 - protein, 17 aa, Artificial Sequence
  Description of Artificial Sequence Synthetic peptide
CIEFGRGNSHYASWAKG

SEQ ID NO: 115 - protein, 32 aa, Artificial Sequence
  Description of Artificial Sequence Synthetic polypeptide
RFTITRSTSLNTVTLQLNSLTAADTATYFCAR

SEQ ID NO: 116 - protein, 11 aa, Artificial Sequence
  Description of Artificial Sequence Synthetic peptide
GSRGVGYGYGL

SEQ ID NO: 117 - protein, 11 aa, Artificial Sequence
  Description of Artificial Sequence Synthetic peptide
WGPGTLVTISS

SEQ ID NO: 118 - DNA, 342 bp, Artificial Sequence
  Description of Artificial Sequence Synthetic polynucleotide
gagctcgatatgacccagactccatcccctgtgtctgcagctgtgggaggcacagtcaccatcaactgccaggccagtcagagtgtttacagtaacagccgcttatcctggtatcagcagaaaccagggcagcctcccaagcaactgatctattctgcatccactctggcatctggggtcccatcgcggttcaagggcagtggatctgggacacatttcactctcaccctcagcggcgtacagtgtgacgatgctgccacttactactgtcaaggcgaatttgcttgtagtagtgctgattgtagtgctttcggcggagggaccgagctggagatcctaaag

SEQ ID NO: 119 - DNA, 69 bp, Artificial Sequence
  Description of Artificial Sequence Synthetic oligonucleotide
gagctcgatatgacccagactccatcccctgtgtctgcagctgtgggaggcacagtcaccatcaactgc

SEQ ID NO: 120 - DNA, 39 bp, Artificial Sequence
  Description of Artificial Sequence Synthetic oligonucleotide
caggccagtcagagtgtttacagtaacagccgcttatcc

SEQ ID NO: 121 - DNA, 45 bp, Artificial Sequence
  Description of Artificial Sequence Synthetic oligonucleotide
tggtatcagcagaaaccagggcagcctcccaagcaactgatctat

SEQ ID NO: 122 - DNA, 21 bp, Artificial Sequence
  Description of Artificial Sequence Synthetic oligonucleotide
tctgcatccactctggcatct

SEQ ID NO: 123 - DNA, 96 bp, Artificial Sequence
  Description of Artificial Sequence Synthetic oligonucleotide
ggggtcccatcgcggttcaagggcagtggatctgggacacatttcactctcaccctcagcggcgtacagtgtgacgatgctgccacttactactgt

SEQ ID NO: 124 - DNA, 39 bp, Artificial Sequence
  Description of Artificial Sequence Synthetic oligonucleotide
caaggcgaatttgcttgtagtagtgctgattgtagtgct

SEQ ID NO: 125 - DNA, 33 bp, Artificial Sequence
  Description of Artificial Sequence Synthetic oligonucleotide
ttcggcggagggaccgagctggagatcctaaag

SEQ ID NO: 126 - protein, 114 aa, Artificial Sequence
  Description of Artificial Sequence Synthetic polypeptide
ELDMTQTPSPVSAAVGGTVTINCQASQSVYSNSRLSWYQQKPGQPPKQLIYSASTLASGVPSRFKGSGSGTHFTLTLSGVQCDDAATYYCQGEFACSSADCSAFGGGTELEILK

SEQ ID NO: 127 - protein, 23 aa, Artificial Sequence
  Description of Artificial Sequence Synthetic peptide
ELDMTQTPSPVSAAVGGTVTINC

SEQ ID NO: 128 - protein, 13 aa, Artificial Sequence
  Description of Artificial Sequence Synthetic peptide
QASQSVYSNSRLS

SEQ ID NO: 129 - protein, 15 aa, Artificial Sequence
  Description of Artificial Sequence Synthetic peptide
WYQQKPGQPPKQLIY

SEQ ID NO: 130 - protein, 7 aa, Artificial Sequence
  Description of Artificial Sequence Synthetic peptide
SASTLAS

SEQ ID NO: 131 - protein, 32 aa, Artificial Sequence
  Description of Artificial Sequence Synthetic polypeptide
GVPSRFKGSGSGTHFTLTLSGVQCDDAATYYC

SEQ ID NO: 132 - protein, 13 aa, Artificial Sequence
  Description of Artificial Sequence Synthetic peptide
QGEFACSSADCSA

SEQ ID NO: 133 - protein, 11 aa, Artificial Sequence
  Description of Artificial Sequence Synthetic peptide
FGGGTELEILK

SEQ ID NO: 134 - DNA, 23 bp, Artificial Sequence
  Description of Artificial Sequence Synthetic primer
cccagtcacgacgttgtaaaacg

SEQ ID NO: 135 - DNA, 23 bp, Artificial Sequence
  Description of Artificial Sequence Synthetic primer
agcggataacaatttcacacagg

SEQ ID NO: 136 - DNA, 42 bp, Artificial Sequence
  Description of Artificial Sequence Synthetic primer
gctgcccaaccagccatggcccagtcggtggaggagtccrgg

SEQ ID NO: 137 - DNA, 42 bp, Artificial Sequence
  Description of Artificial Sequence Synthetic primer
gctgcccaaccagccatggcccagtcggtgaaggagtccgag

SEQ ID NO: 138 - DNA, 42 bp, Artificial Sequence
  Description of Artificial Sequence Synthetic primer
gctgcccaaccagccatggcccagtcgytggaggagtccggg

SEQ ID NO: 139 - DNA, 44 bp, Artificial Sequence
  Description of Artificial Sequence Synthetic primer
gctgcccaaccagccatggcccagsagcagctgrtggagtccgg

SEQ ID NO: 140 - DNA, 24 bp, Artificial Sequence
  Description of Artificial Sequence Synthetic primer
tgargagayggtgaccagggtgcc

SEQ ID NO: 141 - DNA, 38 bp, Artificial Sequence
  Description of Artificial Sequence Synthetic primer
gggcccaggcggccgagctcgtgmtgacccagactcca

SEQ ID NO: 142 - DNA, 38 bp, Artificial Sequence
  Description of Artificial Sequence Synthetic primer
gggcccaggcggccgagctcgatmtgacccagactcca

SEQ ID NO: 143 - DNA, 38 bp, Artificial Sequence
  Description of Artificial Sequence Synthetic primer
gggcccaggcggccgagctcgtgatgacccagactgaa

SEQ ID NO: 144 - DNA, 21 bp, Artificial Sequence
  Description of Artificial Sequence Synthetic primer
taggatctccagctcggtccc

SEQ ID NO: 145 - DNA, 22 bp, Artificial Sequence
  Description of Artificial Sequence Synthetic primer
ttttgatttccacattggtgcc

SEQ ID NO: 146 - DNA, 22 bp, Artificial Sequence
  Description of Artificial Sequence Synthetic primer
ttttgacsaccacctcggtccc

SEQ ID NO: 147 - DNA, 1359 bp, Artificial Sequence
  Description of Artificial Sequence Synthetic polynucleotide
gaggtgcagctggtggagtctggcggaggcctggtgcagccaggcggaagcctgaggctgtcctgcgccgcctcctccgcctctggcttcacaatcaaccggaactactgggtgcggcaggcccctggcaagggcctggagtgggtgagcagcatctacggcggctcctctggaaccacatactacaccaactgggctaagggccgcttcaccatctctagggacaactctaagaacaccctgtacctgcagatgaacagcctgagggccgaggacaccgccgtgtattactgcgccagggtggacaacggaggcgattggtacttcaggctgtggggccagggcacccccgtgacagtgtcttccgcttctaccaagggcccttccgtgttccctctggccccttcctccaagtccacctccggcggcaccgccgctctgggctgcctggtgaaggactacttccctgagcccgtgaccgtgagctggaactctggcgccctgaccagcggagtgcacaccttccctgccgtgctgcagtcctccggcctgtactccctgtcctccgtggtgaccgtgccttcctcctccctgggcacccagacctacatctgcaacgtgaaccacaagccttccaacaccaaggtggacaagaaggtggagcctaagtcctgcgacaagacccacacctgccctccctgccctgcccctgagctgctgggcggaccctccgtgttcctgttccctcctaagcctaaggacaccctgatgatctcccggacccctgaggtgacctgcgtggtggtggacgtgtcccacgaggatcctgaggtgaagttcaattggtacgtggacggcgtggaggtgcacaacgccaagaccaagcctcgggaggaacagtacaactccacctaccgggtggtgtctgtgctgaccgtgctgcaccaggactggctgaacggcaaggaatacaagtgcaaggtctccaacaaggccctgcctgcccccatcgaaaagaccatctccaaggccaagggccagcctcgcgagcctcaggtgtacaccctgcccccctcccgggacgagctgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtctcctggcaagtga

SEQ ID NO: 148 - DNA, 366 bp, Artificial Sequence
  Description of Artificial Sequence Synthetic polynucleotide
gaggtgcagctggtggagtctggcggaggcctggtgcagccaggcggaagcctgaggctgtcctgcgccgcctcctccgcctctggcttcacaatcaaccggaactactgggtgcggcaggcccctggcaagggcctggagtgggtgagcagcatctacggcggctcctctggaaccacatactacaccaactgggctaagggccgcttcaccatctctagggacaactctaagaacaccctgtacctgcagatgaacagcctgagggccgaggacaccgccgtgtattactgcgccagggtggacaacggaggcgattggtacttcaggctgtggggccagggcacccccgtgacagtgtcttcc

SEQ ID NO: 149 - DNA, 75 bp, Artificial Sequence
  Description of Artificial Sequence Synthetic oligonucleotide
gaggtgcagctggtggagtctggcggaggcctggtgcagccaggcggaagcctgaggctgtcctgcgccgcctcc

SEQ ID NO: 150 - DNA, 33 bp, Artificial Sequence
  Description of Artificial Sequence Synthetic oligonucleotide
tccgcctctggcttcacaatcaaccggaactac

SEQ ID NO: 151 - DNA, 42 bp, Artificial Sequence
  Description of Artificial Sequence Synthetic oligonucleotide
tgggtgcggcaggcccctggcaagggcctggagtgggtgagc

SEQ ID NO: 152 - DNA, 54 bp, Artificial Sequence
  Description of Artificial Sequence Synthetic oligonucleotide
agcatctacggcggctcctctggaaccacatactacaccaactgggctaagggc

SEQ ID NO: 153 - DNA, 96 bp, Artificial Sequence
  Description of Artificial Sequence Synthetic oligonucleotide
cgcttcaccatctctagggacaactctaagaacaccctgtacctgcagatgaacagcctgagggccgaggacaccgccgtgtattactgcgccagg

SEQ ID NO: 154 - DNA, 33 bp, Artificial Sequence
  Description of Artificial Sequence Synthetic oligonucleotide
gtggacaacggaggcgattggtacttcaggctg

SEQ ID NO: 155 - DNA, 33 bp, Artificial Sequence
  Description of Artificial Sequence Synthetic oligonucleotide
tggggccagggcacccccgtgacagtgtcttcc

SEQ ID NO: 156 - protein, 452 aa, Artificial Sequence
  Description of Artificial Sequence Synthetic polypeptide
EVQLVESGGGLVQPGGSLRLSCAASSASGFTINRNYWVRQAPGKGLEWVSSIYGGSSGTTYYTNWAKGRFTISRDNSKNTLYLQMNSLRAEDTAVYYCARVDNGGDWYFRLWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 122 aa, Artificial Sequence
  Description of Artificial Sequence Synthetic polypeptide
EVQLVESGGGLVQPGGSLRLSCAASSASGFTINRNYWVRQAPGKGLEWVSSIYGGSSGTTYYTNWAKGRFTISRDNSKNTLYLQMNSLRAEDTAVYYCARVDNGGDWYFRLWGQGTPVTVSS

SEQ ID NO: 158 - protein, 25 aa, Artificial Sequence [sequence omitted]

SEQ ID NO: 159 - protein, 11 aa, Artificial Sequence
  Description of Artificial Sequence Synthetic peptide
SASGFTINRNY

SEQ ID NO: 160 - protein, 14 aa, Artificial Sequence
  Description of Artificial Sequence Synthetic peptide
WVRQAPGKGLEWVS

SEQ ID NO: 161 - protein, 18 aa, Artificial Sequence
  Description of Artificial Sequence Synthetic peptide
SIYGGSSGTTYYTNWAKG

SEQ ID NO: 162 - protein, 32 aa, Artificial Sequence
  Description of Artificial Sequence Synthetic polypeptide
RFTISRDNSKNTLYLQMNSLRAEDTAVYYCAR

SEQ ID NO: 163 - protein, 11 aa, Artificial Sequence
  Description of Artificial Sequence Synthetic peptide
VDNGGDWYFRL

SEQ ID NO: 164 - protein, 11 aa, Artificial Sequence
  Description of Artificial Sequence Synthetic peptide
WGQGTPVTVSS

SEQ ID NO: 165 - DNA, 654 bp, Artificial Sequence
  Description of Artificial Sequence Synthetic polynucleotide
gacatccagatgacacagtccccctccaccctgtctgccagcgtgggcgatagggtgaccatcacatgtcaggccagccagtctatctctagctacctggcctggtaccagcagaagcctggcaaggctcccaagctgctgatctatagggctagcacactggcctctggagtgcctagccggttctctggctccggcagcggaaccgagtttaccctgaccatctcctctctgcagcctgacgacttcgccacctactattgccagcagggcgtgagggtggtggacatcgacaactctttcggccagggaaccaaggtggagatcaagaggaccgtggctgcccccagcgtgttcatcttccccccctctgacgagcagctgaagtccggcacagctagcgtggtgtgcctgctgaacaatttttaccccagagaggctaaggtgcagtggaaggtggacaatgccctgcagagcggcaacagccaggagagcgtgaccgagcaggactccaaggacagcacctactccctgtcttccaccctgacactgagcaaggccgactacgagaagcacaaggtgtacgcttgcgaggtgacccaccagggcctgtcttctcccgtgaccaagtctttcaacaggggcgagtgctga

SEQ ID NO: 166 - DNA, 330 bp, Artificial Sequence
  Description of Artificial Sequence Synthetic polynucleotide
gacatccagatgacacagtccccctccaccctgtctgccagcgtgggcgatagggtgaccatcacatgtcaggccagccagtctatctctagctacctggcctggtaccagcagaagcctggcaaggctcccaagctgctgatctatagggctagcacactggcctctggagtgcctagccggttctctggctccggcagcggaaccgagtttaccctgaccatctcctctctgcagcctgacgacttcgccacctactattgccagcagggcgtgagggtggtggacatcgacaactctttcggccagggaaccaaggtggagatcaag

SEQ ID NO: 167 - DNA, 69 bp, Artificial Sequence
  Description of Artificial Sequence Synthetic oligonucleotide
gacatccagatgacacagtccccctccaccctgtctgccagcgtgggcgatagggtgaccatcacatgt

SEQ ID NO: 168 - DNA, 33 bp, Artificial Sequence
  Description of Artificial Sequence Synthetic oligonucleotide
caggccagccagtctatctctagctacctggcc

SEQ ID NO: 169 - DNA, 45 bp, Artificial Sequence
  Description of Artificial Sequence Synthetic oligonucleotide
tggtaccagcagaagcctggcaaggctcccaagctgctgatctat

SEQ ID NO: 170 - DNA, 21 bp, Artificial Sequence
  Description of Artificial Sequence Synthetic oligonucleotide
agggctagcacactggcctct

SEQ ID NO: 171 - DNA, 96 bp, Artificial Sequence
  Description of Artificial Sequence Synthetic oligonucleotide
ggagtgcctagccggttctctggctccggcagcggaaccgagtttaccctgaccatctcctctctgcagcctgacgacttcgccacctactattgc

SEQ ID NO: 172 - DNA, 36 bp, Artificial Sequence
  Description of Artificial Sequence Synthetic oligonucleotide
cagcagggcgtgagggtggtggacatcgacaactct

SEQ ID NO: 173 - DNA, 36 bp, Artificial Sequence; identical to SEQ ID NO: 172
  Description of Artificial Sequence Synthetic oligonucleotide

SEQ ID NO: 174 - protein, 217 aa, Artificial Sequence
  Description of Artificial Sequence Synthetic polypeptide
DIQMTQSPSTLSASVGDRVTITCQASQSISSYLAWYQQKPGKAPKLLIYRASTLASGVPSRFSGSGSGTEFTLTISSLQPDDFATYYCQQGVRVVDIDNSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 111 aa, Artificial Sequence
  Description of Artificial Sequence Synthetic polypeptide
DIQMTQSPSTLSASVGDRVTITCQASQSISSYLAWYQQKPGKAPKLLIYRASTLASGVPSRFSGSGSGTEFTLTISSLQPDDFATYYCQQGVRVVDIDNSFGQGTKVEIKR

SEQ ID NO: 176 - protein, 23 aa, Artificial Sequence
  Description of Artificial Sequence Synthetic peptide
DIQMTQSPSTLSASVGDRVTITC

SEQ ID NO: 177 - protein, 11 aa, Artificial Sequence
  Description of Artificial Sequence Synthetic peptide
QASQSISSYLA

SEQ ID NO: 178 - protein, 15 aa, Artificial Sequence
  Description of Artificial Sequence Synthetic peptide
WYQQKPGKAPKLLIY

SEQ ID NO: 179 - protein, 7 aa, Artificial Sequence
  Description of Artificial Sequence Synthetic peptide
RASTLAS

SEQ ID NO: 180 - protein, 32 aa, Artificial Sequence
  Description of Artificial Sequence Synthetic polypeptide
GVPSRFSGSGSGTEFTLTISSLQPDDFATYYC

SEQ ID NO: 181 - protein, 12 aa, Artificial Sequence
  Description of Artificial Sequence Synthetic peptide
QQGVRVVDIDNS

SEQ ID NO: 182 - protein, 11 aa, Artificial Sequence
  Description of Artificial Sequence Synthetic peptide
FGQGTKVEIKR

SEQ ID NO: 183 - DNA, 2796 bp, Artificial Sequence
  Description of Artificial Sequence Synthetic polynucleotide
atgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccgggttaacgacgacgacgacaaagagcccagcggaccaatttcaacaatcaacccctctcctccatccaaggagtctcacaaaagcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagcycagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatga

SEQ ID NO: 184 - DNA, 1470 bp, Artificial Sequence
  Description of Artificial Sequence Synthetic polynucleotide
atgggatggagctgtatcatcctcttcttggtagcaacagctacaggtgtccactccgaggtgcagctggtggagtctgggggaggcttggtccagcctggggggtccctgagactctcctgtgcagcctctggattctccttcagtagcagctactggggatgccgccaggctccagggaaggggctggagtgggtctcatgcatttatggtggtagtagtggtaccacttattacccgaactgggcgaaaggcagattcaccatctccagagacaattccaagaacacgctgtatcttcaaatgaacagcctgagagccgaggacacggctgtgtattactgtgcgagagtgactctgcaaatggccagtctgacagccgcggacacggccacctatttctgtgcgagagtgactaatggtggtgattgggattttaaattgtggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 185 - protein, 489 aa, Artificial Sequence
  Description of Artificial Sequence Synthetic polypeptide
MGWSCIILFLVATATGVHSEVQLVESGGGLVQPGGSLRLSCAASGFSFSSSYWGCRQAPGKGLEWVSCIYGGSSGTTYYPNWAKGRFTISRDNSKNTLYLQMNSLRAEDTAVYYCARVTLQMASLTAADTATYFCARVTNGGDWDFKLWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - DNA, 711 bp, Artificial Sequence
  Description of Artificial Sequence Synthetic polynucleotide
atgggatggagctgtatcatcctcttcttggtagcaacagctacaggtgtccactccgacatccagatgacccagtctccatcctccctgtctgcatctgtaggagacagagtcaccatcacttgccaggccagtcagaccattaataactacttgacctggtatcagcagaaaccagggaaagttcctaagctcctgatctatagggcatccactctggcatctggggtcccatctcggttcagtggcagtggatctgggacagatttcactctcaccatcagcagcctgcagcctgaagatgttgcaacttattactgtcaacagactgttcgtgtggttgatgttgataatagtttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 187 - protein, 236 aa, Artificial Sequence
  Description of Artificial Sequence Synthetic polypeptide
MGWSCIILFLVATATGVHSDIQMTQSPSSLSASVGDRVTITCQASQTINNYLTWYQQKPGKVPKLLIYRASTLASGVPSRFSGSGSGTDFTLTISSLQPEDVATYYCQQTVRVVDVDNSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - DNA, 1476 bp, Artificial Sequence
  Description of Artificial Sequence Synthetic polynucleotide
atgggatggagctgtatcatcctcttcttggtagcaacagctacaggtgtccactccgaggtgcagctggtggagtctgggggaggcttggtccagcctggggggtccctgagactctcctgtgcagcctctggattctccttcagtagcagctactggggatgctgggtccgccaggctccagggaaggggctggagtgggtctcatgcatttatggtggtagtagtggtaccacttattacccgaactgggcgaaaggcagattcaccatctccagagacaattccaagaacacgctgtatcttcaaatgaacagcctgagagccgaggacacggctgtgtattactgtgcgagagtgactctgcaaatggccagtctgacagccgcggacacggccacctatttctgtgcgagagtgactaatggtggtgattgggattttaaattgtggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 189 - protein, 491 aa, Artificial Sequence
  Description of Artificial Sequence Synthetic polypeptide
MGWSCIILFLVATATGVHSEVQLVESGGGLVQPGGSLRLSCAASGFSFSSSYWGCWVRQAPGKGLEWVSCIYGGSSGTTYYPNWAKGRFTISRDNSKNTLYLQMNSLRAEDTAVYYCARVTLQMASLTAADTATYFCARVTNGGDWDFKLWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - DNA, 711 bp, Artificial Sequence
  Description of Artificial Sequence Synthetic polynucleotide
atgggatggagctgtatcatcctcttcttggtagcaacagctacaggtgtccactccgacatccagatgacccagtctccatcctccctgtctgcatctgtaggagacagagtcaccatcacttgccaggccagtcagagcattaatacctacttagcctggtatcagcagaaaccagggaaagttcctaagctcctgatctatagggcatccactctggcatctggggtcccatctcggttcagtggcagtggatctgggacagatttcactctcaccatcagcagcctgcagcctgaagatgttgcaacttattactgtcaacagagtgttcgtgttattgatgttgataatactttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 191 - protein, 236 aa, Artificial Sequence
  Description of Artificial Sequence Synthetic polypeptide
MGWSCIILFLVATATGVHSDIQMTQSPSSLSASVGDRVTITCQASQSINTYLAWYQQKPGKVPKLLIYRASTLASGVPSRFSGSGSGTDFTLTISSLQPEDVATYYCQQSVRVIDVDNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - DNA, 420 bp, Artificial Sequence
  Description of Artificial Sequence Synthetic polynucleotide
gaggtgcagctggtggagtctgggggaggcttggtccagcctggggggtccctgagactctcctgtgcagcctctggattctccttcagtagcagctactggggatgccgccaggctccagggaaggggctggagtgggtctcatgcatttatggtggtagtagtggtaccacttattacccgaactgggcgaaaggcagattcaccatctccagagacaattccaagaacacgctgtatcttcaaatgaacagcctgagagccgaggacacggctgtgtattactgtgcgagagtgactctgcaaatggccagtctgacagccgcggacacggccacctatttctgtgcgagagtgactaatggtggtgattgggattttaaattgtggggccaagggaccccggtcaccgtctcctca

SEQ ID NO: 193 - DNA, 75 bp, Artificial Sequence
  Description of Artificial Sequence Synthetic oligonucleotide
gaggtgcagctggtggagtctgggggaggcttggtccagcctggggggtccctgagactctcctgtgcagcctct

SEQ ID NO: 194 - DNA, 30 bp, Artificial Sequence
  Description of Artificial Sequence Synthetic oligonucleotide
ggattctccttcagtagcagctactgggga

SEQ ID NO: 195 - DNA, 39 bp, Artificial Sequence
  Description of Artificial Sequence Synthetic oligonucleotide
tgccgccaggctccagggaaggggctggagtgggtctca

SEQ ID NO: 196 - DNA, 54 bp, Artificial Sequence; identical to SEQ ID NO: 42
  Description of Artificial Sequence Synthetic oligonucleotide

SEQ ID NO: 197 - DNA, 156 bp, Artificial Sequence
  Description of Artificial Sequence Synthetic polynucleotide
agattcaccatctccagagacaattccaagaacacgctgtatcttcaaatgaacagcctgagagccgaggacacggctgtgtattactgtgcgagagtgactctgcaaatggccagtctgacagccgcggacacggccacctatttctgtgcgaga

SEQ ID NO: 198 - DNA, 33 bp, Artificial Sequence
  Description of Artificial Sequence Synthetic oligonucleotide
gtgactaatggtggtgattgggattttaaattg

SEQ ID NO: 199 - DNA, 33 bp, Artificial Sequence
  Description of Artificial Sequence Synthetic oligonucleotide
tggggccaagggaccccggtcaccgtctcctca

SEQ ID NO: 200 - protein, 140 aa, Artificial Sequence
  Description of Artificial Sequence Synthetic polypeptide
EVQLVESGGGLVQPGGSLRLSCAASGFSFSSSYWGCRQAPGKGLEWVSCIYGGSSGTTYYPNWAKGRFTISRDNSKNTLYLQMNSLRAEDTAVYYCARVTLQMASLTAADTATYFCARVTNGGDWDFKLWGQGTPVTVSS

SEQ ID NO: 201 - protein, 25 aa, Artificial Sequence [sequence omitted]

SEQ ID NO: 202 - protein, 10 aa, Artificial Sequence
  Description of Artificial Sequence Synthetic peptide
GFSFSSSYWG

SEQ ID NO: 203 - protein, 13 aa, Artificial Sequence
  Description of Artificial Sequence Synthetic peptide
CRQAPGKGLEWVS

SEQ ID NO: 204 - protein, 18 aa, Artificial Sequence
  Description of Artificial Sequence Synthetic peptide
CIYGGSSGTTYYPNWAKG

SEQ ID NO: 205 - protein, 52 aa, Artificial Sequence
  Description of Artificial Sequence Synthetic polypeptide
RFTISRDNSKNTLYLQMNSLRAEDTAVYYCARVTLQMASLTAADTATYFCAR

SEQ ID NO: 206 - protein, 11 aa, Artificial Sequence
  Description of Artificial Sequence Synthetic peptide
VTNGGDWDFKL

SEQ ID NO: 207 - protein, 11 aa, Artificial Sequence
  Description of Artificial Sequence Synthetic peptide
WGQGTPVTVSS

SEQ ID NO: 208 - DNA, 330 bp, Artificial Sequence
  Description of Artificial Sequence Synthetic polynucleotide
gacatccagatgacccagtctccatcctccctgtctgcatctgtaggagacagagtcaccatcacttgccaggccagtcagaccattaataactacttgacctggtatcagcagaaaccagggaaagttcctaagctcctgatctatagggcatccactctggcatctggggtcccatctcggttcagtggcagtggatctgggacagatttcactctcaccatcagcagcctgcagcctgaagatgttgcaacttattactgtcaacagactgttcgtgtggttgatgttgataatagtttcggccaagggaccaaggtggaaatcaaa

SEQ ID NO: 209 - DNA, 69 bp, Artificial Sequence
  Description of Artificial Sequence Synthetic oligonucleotide
gacatccagatgacccagtctccatcctccctgtctgcatctgtaggagacagagtcaccatcacttgc

SEQ ID NO: 210 - DNA, 33 bp, Artificial Sequence; identical to SEQ ID NO: 56
  Description of Artificial Sequence Synthetic oligonucleotide

SEQ ID NO: 211 - DNA, 45 bp, Artificial Sequence
  Description of Artificial Sequence Synthetic oligonucleotide
tggtatcagcagaaaccagggaaagttcctaagctcctgatctat

SEQ ID NO: 212 - DNA, 21 bp, Artificial Sequence
  Description of Artificial Sequence Synthetic oligonucleotide
agggcatccactctggcatct

SEQ ID NO: 213 - DNA, 96 bp, Artificial Sequence
  Description of Artificial Sequence Synthetic oligonucleotide
ggggtcccatctcggttcagtggcagtggatctgggacagatttcactctcaccatcagcagcctgcagcctgaagatgttgcaacttattactgt

SEQ ID NO: 214 - DNA, 36 bp, Artificial Sequence; identical to SEQ ID NO: 60
  Description of Artificial Sequence Synthetic oligonucleotide

SEQ ID NO: 215 - DNA, 30 bp, Artificial Sequence
  Description of Artificial Sequence Synthetic oligonucleotide
ttcggccaagggaccaaggtggaaatcaaa

SEQ ID NO: 216 - protein, 110 aa, Artificial Sequence
  Description of Artificial Sequence Synthetic polypeptide
DIQMTQSPSSLSASVGDRVTITCQASQTINNYLTWYQQKPGKVPKLLIYRASTLASGVPSRFSGSGSGTDFTLTISSLQPEDVATYYCQQTVRVVDVDNSFGQGTKVEIK

SEQ ID NO: 217 - protein, 23 aa, Artificial Sequence
  Description of Artificial Sequence Synthetic peptide
DIQMTQSPSSLSASVGDRVTITC

SEQ ID NO: 218 - protein, 11 aa, Artificial Sequence
  Description of Artificial Sequence Synthetic peptide
QASQTINNYLT

SEQ ID NO: 219 - protein, 15 aa, Artificial Sequence
  Description of Artificial Sequence Synthetic peptide
WYQQKPGKVPKLLIY

SEQ ID NO: 220 - protein, 7 aa, Artificial Sequence
  Description of Artificial Sequence Synthetic peptide
RASTLAS

SEQ ID NO: 221 - protein, 32 aa, Artificial Sequence
  Description of Artificial Sequence Synthetic polypeptide
GVPSRFSGSGSGTDFTLTISSLQPEDVATYYC

SEQ ID NO: 222 - protein, 12 aa, Artificial Sequence
  Description of Artificial Sequence Synthetic peptide
QQTVRVVDVDNS

SEQ ID NO: 223 - protein, 10 aa, Artificial Sequence
  Description of Artificial Sequence Synthetic peptide
FGQGTKVEIK

SEQ ID NO: 224 - DNA, 426 bp, Artificial Sequence
  Description of Artificial Sequence Synthetic polynucleotide
gaggtgcagctggtggagtctgggggaggcttggtccagcctggggggtccctgagactctcctgtgcagcctctggattctccttcagtagcagctactggggatgctgggtccgccaggctccagggaaggggctggagtgggtctcatgcatttatggtggtagtagtggtaccacttattacccgaactgggcgaaaggcagattcaccatctccagagacaattccaagaacacgctgtatcttcaaatgaacagcctgagagccgaggacacggctgtgtattactgtgcgagagtgactctgcaaatggccagtctgacagccgcggacacggccacctatttctgtgcgagagtgactaatggtggtgattgggattttaaattgtggggccaagggaccccggtcaccgtctcctca

SEQ ID NO: 225 - DNA, 75 bp, Artificial Sequence; identical to SEQ ID NO: 193
  Description of Artificial Sequence Synthetic oligonucleotide

SEQ ID NO: 226 - DNA, 33 bp, Artificial Sequence; identical to SEQ ID NO: 40
  Description of Artificial Sequence Synthetic oligonucleotide

SEQ ID NO: 227 - DNA, 42 bp, Artificial Sequence
  Description of Artificial Sequence Synthetic oligonucleotide
tgggtccgccaggctccagggaaggggctggagtgggtctca

SEQ ID NO: 228 - DNA, 54 bp, Artificial Sequence; identical to SEQ ID NO: 42
  Description of Artificial Sequence Synthetic oligonucleotide

SEQ ID NO: 229 - DNA, 156 bp, Artificial Sequence; identical to SEQ ID NO: 197
  Description of Artificial Sequence Synthetic polynucleotide

SEQ ID NO: 230 - DNA, 33 bp, Artificial Sequence; identical to SEQ ID NO: 198
  Description of Artificial Sequence Synthetic oligonucleotide

SEQ ID NO: 231 - DNA, 33 bp, Artificial Sequence; identical to SEQ ID NO: 199
  Description of Artificial Sequence Synthetic oligonucleotide

SEQ ID NO: 232 - protein, 142 aa, Artificial Sequence
  Description of Artificial Sequence Synthetic polypeptide
EVQLVESGGGLVQPGGSLRLSCAASGFSFSSSYWGCWVRQAPGKGLEWVSCIYGGSSGTTYYPNWAKGRFTISRDNSKNTLYLQMNSLRAEDTAVYYCARVTLQMASLTAADTATYFCARVTNGGDWDFKLWGQGTPVTVSS

SEQ ID NO: 233 - protein, 25 aa, Artificial Sequence [sequence omitted]

SEQ ID NO: 234 - protein, 11 aa, Artificial Sequence
  Description of Artificial Sequence Synthetic peptide
GFSFSSSYWGC

SEQ ID NO: 235 - protein, 14 aa, Artificial Sequence
  Description of Artificial Sequence Synthetic peptide
WVRQAPGKGLEWVS

SEQ ID NO: 236 - protein, 18 aa, Artificial Sequence
  Description of Artificial Sequence Synthetic peptide
CIYGGSSGTTYYPNWAKG

SEQ ID NO: 237 - protein, 52 aa, Artificial Sequence; identical to SEQ ID NO: 205
  Description of Artificial Sequence Synthetic polypeptide

SEQ ID NO: 238 - protein, 11 aa, Artificial Sequence
  Description of Artificial Sequence Synthetic peptide
VTNGGDWDFKL

SEQ ID NO: 239 - protein, 11 aa, Artificial Sequence
  Description of Artificial Sequence Synthetic peptide
WGQGTPVTVSS

SEQ ID NO: 240 - DNA, 330 bp, Artificial Sequence
  Description of Artificial Sequence Synthetic polynucleotide
gacatccagatgacccagtctccatcctccctgtctgcatctgtaggagacagagtcaccatcacttgccaggccagtcagagcattaatacctacttagcctggtatcagcagaaaccagggaaagttcctaagctcctgatctatagggcatccactctggcatctggggtcccatctcggttcagtggcagtggatctgggacagatttcactctcaccatcagcagcctgcagcctgaagatgttgcaacttattactgtcaacagagtgttcgtgttattgatgttgataatactttcggccaagggaccaaggtggaaatcaaa

SEQ ID NO: 241 - DNA, 69 bp, Artificial Sequence; identical to SEQ ID NO: 209
  Description of Artificial Sequence Synthetic oligonucleotide

SEQ ID NO: 242 - DNA, 33 bp, Artificial Sequence; identical to SEQ ID NO: 88
  Description of Artificial Sequence Synthetic oligonucleotide

SEQ ID NO: 243 - DNA, 45 bp, Artificial Sequence; identical to SEQ ID NO: 211
  Description of Artificial Sequence Synthetic oligonucleotide

SEQ ID NO: 244 - DNA, 21 bp, Artificial Sequence
  Description of Artificial Sequence Synthetic oligonucleotide
agggcatccactctggcatct

SEQ ID NO: 245 - DNA, 96 bp, Artificial Sequence; identical to SEQ ID NO: 213
  Description of Artificial Sequence Synthetic oligonucleotide

SEQ ID NO: 246 - DNA, 36 bp, Artificial Sequence; identical to SEQ ID NO: 92
  Description of Artificial Sequence Synthetic oligonucleotide

SEQ ID NO: 247 - DNA, 30 bp, Artificial Sequence; identical to SEQ ID NO: 215
  Description of Artificial Sequence Synthetic oligonucleotide

SEQ ID NO: 248 - protein, 110 aa, Artificial Sequence
  Description of Artificial Sequence Synthetic polypeptide
DIQMTQSPSSLSASVGDRVTITCQASQSINTYLAWYQQKPGKVPKLLIYRASTLASGVPSRFSGSGSGTDFTLTISSLQPEDVATYYCQQSVRVIDVDNTFGQGTKVEIK

SEQ ID NO: 249 - protein, 23 aa, Artificial Sequence
  Description of Artificial Sequence Synthetic peptide
DIQMTQSPSSLSASVGDRVTITC

SEQ ID NO: 250 - protein, 11 aa, Artificial Sequence
  Description of Artificial Sequence Synthetic peptide
QASQSINTYLA

SEQ ID NO: 251 - protein, 15 aa, Artificial Sequence
  Description of Artificial Sequence Synthetic peptide
WYQQKPGKVPKLLIY

SEQ ID NO: 252 - protein, 7 aa, Artificial Sequence
  Description of Artificial Sequence Synthetic peptide
RASTLAS

SEQ ID NO: 253 - protein, 32 aa, Artificial Sequence; identical to SEQ ID NO: 221
  Description of Artificial Sequence Synthetic polypeptide

SEQ ID NO: 254 - protein, 12 aa, Artificial Sequence
  Description of Artificial Sequence Synthetic peptide
QQSVRVIDVDNT

SEQ ID NO: 255 - protein, 10 aa, Artificial Sequence
  Description of Artificial Sequence Synthetic peptide
FGQGTKVEIK

SEQ ID NO: 256 - DNA, 33 bp, Artificial Sequence
  Description of Artificial Sequence Synthetic primer
gtcatctagacacgctggttctgcaggtctgca

SEQ ID NO: 257 - DNA, 35 bp, Artificial Sequence
  Description of Artificial Sequence Synthetic primer
gatcgaattcaccatgctgctgcgcctgttgctgg

SEQ ID NO: 258 - DNA, 58 bp, Artificial Sequence
  Description of Artificial Sequence Synthetic primer
cagtgttaacgacgacgacgacaaagagcccagcggaccaatttcaacaatcaacccc

SEQ ID NO: 259 - DNA, 35 bp, Artificial Sequence
  Description of Artificial Sequence Synthetic primer
gggttcgaatcatttacccggagaccgggagatgg

SEQ ID NO: 260 - protein, 15 aa, Artificial Sequence
  Description of Artificial Sequence Synthetic peptide
GGGGSGGGGSGGGGS

(19) United States
(12) Patent Application Publication
(10) Pub. No.: US 2010/0260769 A1
(43) Pub. Date: Oct. 14, 2010

(54) ENDOSIALIN BINDING MOLECULES

(75) Inventors: Philip M. Sass, Audubon, PA (US); Brad Kline, Morgantown, PA (US); Nicholas Nicolaides, Glen Mills, PA (US); Luigi Grasso, Bryn Mawr, PA (US); Stephen Harley, Morgantown, PA (US)

Correspondence Address: ROPES & GRAY LLP, PATENT DOCKETING 39/361, 1211 AVENUE OF THE AMERICAS, NEW YORK, NY, 10036-8704, US

(73) Assignee: MORPHOTEK, INC., EXTON, PA (US)

(21) Appl. No.: 12/756,353

(22) Filed: Apr. 8, 2010

Related U.S. Application Data
(60) Provisional application No. 61/167,994, filed on Apr. 9, 2009.

(51) Int. Cl. C07K 16/30; C12N 5/16; C12N 9/96; G01N 33/574; C12N 15/13; C12P 21/08; C12N 15/85; A61K 39/395; A61P 35/04

(52) U.S. Cl. 424/156.1; 530/388.85; 530/387.3; 435/344.1; 530/391.3; 435/188; 435/7.23; 536/23.53; 435/320.1; 435/325; 435/69.6

(57) ABSTRACT

The invention relates to novel binding molecules that specifically bind endosialin (TEM-1) including antibodies, such as monoclonal antibodies, or antigen-binding portions of antibodies, and methods and compositions comprising such binding molecules.

This application claims priority under 35 U.S.C. §119(e) from U.S. provisional application 61/167,994, filed Apr. 9, 2009. The contents of 61/167,994 are hereby incorporated by reference in their entirety.

The instant application contains a Sequence Listing which has been submitted via EFS-Web as an ASCII text file in lieu of a paper copy and is hereby incorporated by reference in its entirety. Said ASCII text file, created on Mar. 9, 2010, is named Sequence_Listing.txt, and is 139,299 bytes in size.

### FIELD OF THE INVENTION

The present invention relates to endosialin/TEM-1 binding molecules, including antibodies and antigen-binding portions of antibodies. The invention also relates to nucleic acid molecules encoding such binding molecules, vectors and host cells comprising such nucleic acid molecules and methods of making such binding molecules. The invention further relates to compositions, kits and tissue samples comprising such binding molecules and methods of using such binding molecules and compositions.

### BACKGROUND OF THE INVENTION

Endosialin/TEM1 is a cell surface protein that was originally discovered as a human embryonic fibroblast-specific antigen and was later found to be differentially expressed in tumor stroma and endothelium. Endosialin/TEM1 is a C-type, lectin-like, integral membrane receptor exhibiting a high degree of O-linked glycosylation (Christian et al., J Biol Chem. 2001; 276: 48588-48595).

Endosialin/TEM1 overexpression has been observed in many cancers of various tissue origin, including colon, breast, pancreatic, and lung. The knockout (KO) mouse model showed the absence of endosialin/TEM1 expression reduced growth, invasion, and metastasis of human tumor xenografts. In addition, lack of endosialin/TEM1 led to an increase in small immature blood vessels and decreased numbers of medium and large tumor vessels. Cells expressing endosialin/TEM1 exhibit enhanced adhesion to Fibronectin and enhanced migration through matrigel, although these properties can be blocked by a humanized mouse antibody directed against human endosialin/TEM1.

Accordingly, there is an urgent need for endosialin/TEM-1 binding molecules with improved properties for detecting endosialin in cells or tissue in or from a subject and for antagonist endosialin binding molecules with improved properties for therapy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph depicting the growth of tumor xenografts in athymic NCr-nu/nu nude mice using A431 cells and A431 cells transformed with human endosialin/TEM1 cDNA.

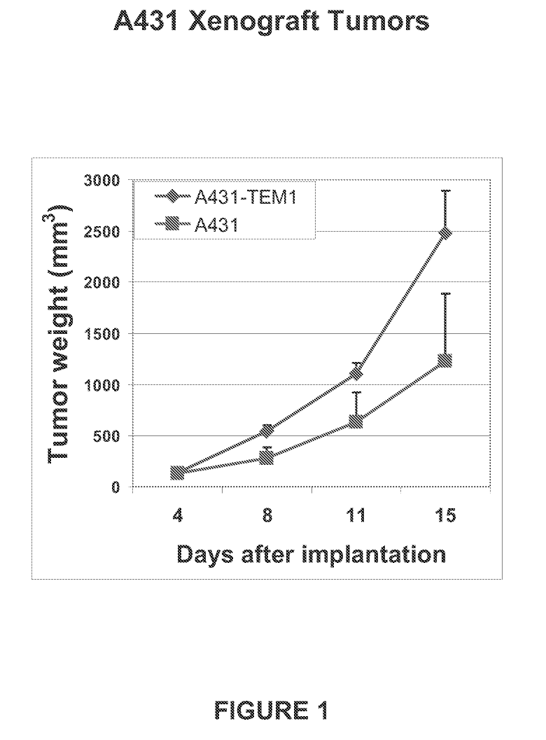

FIG. 2 provides photographs of frozen sections of tumor xenografts formed from A431 cells transformed with endosialin/TEM1 (FIGS. 2A and 2B) and untransformed A431 cells (FIGS. 2C and 2D) stained with a biotinylated humanized anti-endosialin/TEM1 antibody (MORAb-004) (FIGS. 2A and 2C) or with a biotinylated human IgG control antibody (20 μg/mL) (FIGS. 2B and 2D).

FIG. 3 provides photographs of formalin-fixed, paraffin-embedded (FFPE) of A431-TEM1 (FIGS. 3A and 3B) and A431 (FIGS. 3C and 3D) tumor xenografts stained with biotinylated rabbit monoclonal antibody 1-55-2 (20 μg/ml) or a biotinylated control rabbit antibody. FIG. 3A shows extensive staining.

FIG. 4 is a bar graph showing inhibition of endosialin/TEM1 binding to fibronectin by 3 rabbit monoclonal antibodies of the invention, MORAb-004 and an irrelevant antibody (control).

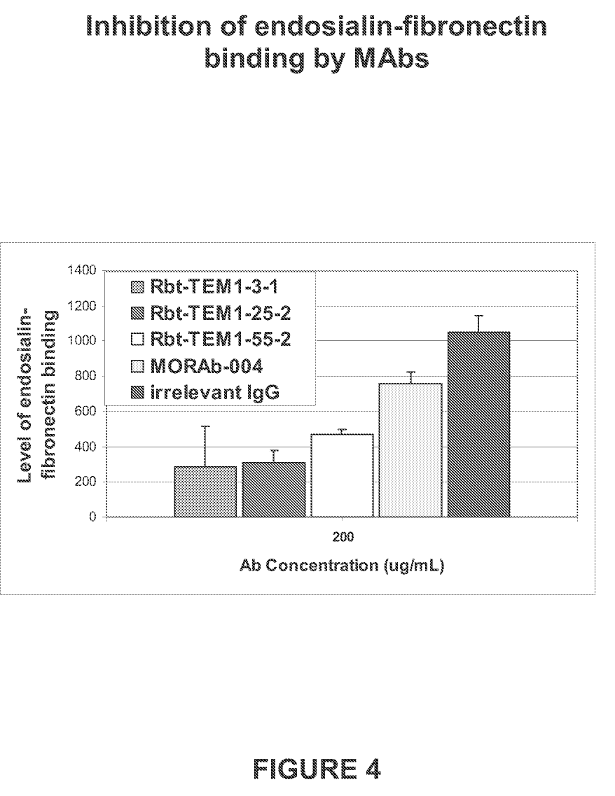

FIG. 5 is a series of graphs showing dose/response curves for inhibition of endosialin binding to fibronectin by anti-endosialin antibodies of the invention and the calculated IC₅₀s.

### SUMMARY OF THE INVENTION

In one aspect, the invention provides a rabbit monoclonal antibody or an antigen-binding portion thereof that specifically binds endosialin, compositions including pharmaceutical compositions comprising one or more of such antibodies, and methods for making and using such antibodies or antigen-binding portions. In various embodiments, such antibody or antigen-binding portion is an endosialin antagonist, reduces fibronectin binding to endosialin, in some cases with an IC₅₀ of 75 μg/mL or lower, reduces endosialin-mediated angiogenesis, reduces growth in an endosialin-expressing tumor in vitro or in vivo, reduces proliferation of human epidermoid cancer cells, reduces endosialin-mediated signaling and/or does not bind endosialin in the lectin domain. In some embodiments, antigen-binding portion is useful for immunohistochemistry (IHC) staining, including in a frozen tissue sample or in a formalin-fixed, paraffin-embedded tissue sample. In some embodiments, antigen-binding portion stains a formalin-fixed, paraffin-embedded tissue sample at least twice the visual intensity of non-specific, background control staining. Exemplary rabbit monoclonal antibodies that bind human endosialin/TEM-1 include antibody 1-55-2, antibody 1-3-1 and antibody 1-25-2. An exemplary rabbit monoclonal antibody that binds mouse endosialin/TEM-1 is clone 8.

In some embodiments, the anti-endosialin/TEM-1 antibody is a humanized antibody comprising a human immunoglobulin heavy chain constant region, wherein said constant region is selected from the group consisting of: a human IgM constant region, a human IgG1 constant region, a human IgG2 constant region, a human IgG3 constant region, a human IgG4 constant region, a human IgE constant region A1 and a human IgA2M constant region. Examples of such humanized antibodies are humanized 1-55-2 (Hu 1-55-2), Hu 1-25-2 and Hu 1-3-1.

The invention also provides an antibody or an antigen-binding portion thereof that specifically binds endosialin and binds the same epitope as, competes for binding to endosialin with and/or cross-competes for binding to endosialin with one or more of antibody 1-55-2, 1-3-1, 1-25-2, Hu 1-55-2, Hu 1-3-1, Hu 1-25-2 or clone 8.

In various embodiments, the rabbit monoclonal antibody or antigen-binding portion internalizes in endosialin-expressing cells, exhibits antibody dependent cellular cytotoxicity (ADCC), exhibits complement-dependent cytotoxicity (CDC), and/or stains a formalin-fixed, paraffin embedded tissue sample with substantially the same intensity as antibody 1-55-2, 1-3-1, 1-25-2, Hu 1-55-2, Hu 1-3-1 or Hu 1-25-2. In some embodiments, the antibody or portion is detectably labeled.

In another aspect, the invention provides an immunohistochemistry assay kit comprising an antibody or antigen-binding portion of the invention.

In another aspect, the invention provides a nucleic acid molecule that encodes one or more components of an antibody of the invention, a vector comprising such nucleic acid and host cells comprising the nucleic acid and/or vector.

### DETAILED DESCRIPTION OF THE INVENTION

The invention provides endosialin binding molecules including antibodies or antigen-binding portions of antibodies that specifically bind endosialin (or a portion of endosialin, such as the extracellular domain (ECD)) and are useful for therapy and diagnosis. In particular, the invention provides endosialin binding molecules that antagonize at least one biological activity of endosialin, especially ones that reduce angiogenesis in a tumor. Also provided are endosialin binding molecules that specifically bind endosialin on cells and in tissues, particularly in tissue samples such as formalin-fixed, paraffin-embedded (FFPE) tissues. The endosialin binding molecules of the invention are useful to detect endosialin in cells and tissues in vivo and in vitro, for diagnosing a subject with a condition characterized by unwanted endosialin expression, for identifying subjects who are candidates for therapy with an antagonist endosialin binding molecule including an antibody that specifically binds endosialin or an antigen-binding portion of such an antibody, methods for reducing one or more activity of endosialin including reducing endosialin-mediated angiogenesis in a subject in need thereof.

The term “isolated protein”, “isolated polypeptide” or “isolated antibody” is a protein, polypeptide or antibody that by virtue of its origin or source of derivation (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally-associated components by isolation, using protein purification techniques well known in the art.

Examples of isolated antibodies include an anti-endosialin antibody that has been affinity purified using endosialin or a portion thereof, an anti-endosialin antibody that has been synthesized by a hybridoma or other cell line in vitro and a humanized anti-endosialin antibody synthesized in vitro.

A protein or polypeptide is “substantially pure,” “substantially homogeneous,” or “substantially purified” when at least about 60 to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and may be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

In particular, the invention provides rabbit anti-endosialin antibodies as well as chimeric and humanized antibodies comprising one or more CDR regions from a rabbit anti-endosialin antibody. The endosialin can be any mammalian endosialin. Of particular interest, however, are specific binding molecules including antibodies that bind human endosialin (SEQ ID NO: 2). The invention also encompasses endosialin binding molecules comprising a portion of a rabbit anti-endosialin antibody, such as a CDR region on a non-immunoglobulin scaffold.

The antigen-binding domains of the anti-endosialin antibodies of the invention may be incorporated into a biocompatible non-immunoglobulin scaffold or framework (see, for example, Binz et al. (2005) Nat Biotech 23: 1257-1268; Hosse et al. (2006) Protein Science 15: 14-27), incorporated by reference in their entirety). In one embodiment, the scaffold or framework may display the antigen-binding amino acid sequence in a localized surface region. These scaffolds or frameworks may be based on fibronectin, ankyrin, lipocalin, neocarzinostain, cytochrome b, CPI zinc finger, PSTI, coiled coil, LACI-DI, Z domain and tendramisat domains (see e.g., Nygren and Uhlen (1997) Current Opinion in Structural Biology, 7: 463-469, incorporated by reference in its entirety). An antibody of the invention may be polyclonal or monoclonal. As used herein a monoclonal antibody refers to an antibody produced by a single cell or a clonal population of cells. The cell (or cells) can be a B cell or a host cell expressing the antibody. In some cases, the cell is an immortalized cell.

The invention provides complete antibodies and antigen-binding portions of antibodies. For the purposes of this application the mature light chain and heavy chain variable domain comprise three complementarity determining regions (CDR1, CDR2 and CDR3) within four framework regions (FR1, FR2, FR3 and FR4) arranged from N-terminus to C-terminus FR1, CDR1, FR2, CDR2, FR3, CDR3 and FR4. The assignment of amino acids to each domain herein is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987) or Chothia et al., Nature 342:878-883 (1989). Heavy chain constant regions comprise a CH1 domain and hinge, followed by two CH regions. Embodiments of the invention comprising an antibody heavy chain constant region may be of any isotype. Where the constant region is a human heavy chain constant region, the isotype may be any of IgM, IgD, IgG1, IgG2, IgG3, IgG4, IgE, IgA1 or IgA2. Where the constant region is a rabbit heavy chain constant region, it may be of any of IgG, IgA (including any sub-isotype, for example IgA1, IgA2, IgA3, IgA4, IgA5, IgA6, IgA7, IgA8, IgA9, IgA10, IgA11, IgA12 or IgA13), IgM or IgE. Embodiments comprising a light chain constant region may be kappa (K) or lambda (A) light chains.

An antigen-binding portion of an anti-endosialin antibody of the invention is any portion that competes with the intact antibody for specific binding. See generally, Fundamental Immunology, Ch. 7 (Paul, W., ed., second ed. Raven Press, N.Y. (1989)) (incorporated by reference in its entirety for all purposes). Antigen-binding portions may be produced by recombinant DNA techniques or by enzymatic or chemical cleavage of intact antibodies. In some embodiments, antigen-binding portions include Fab, Fab′, F(ab′)₂, Fd, Fv, domain antibodies (dAb), complementarity determining region (CDR) fragments, single-chain antibodies (scFv), diabodies and polypeptides that contain at least a portion of an antibody that is sufficient to confer specific antigen binding to the polypeptide.

An anti-endosialin antibody of the invention may be a chimeric antibody, i.e., an antibody that comprises regions from two or more different antibodies that may be of the same or of different species. A chimeric anti-endosialin antibody, thus, may have components from two or more different rabbit antibodies or may have components from one or more rabbit antibodies and components from one or more antibodies of another species, e.g., from one or more non-human primate antibodies or from one or more human antibodies or any combination of the foregoing. A chimeric anti-endosialin antibody comprising human antibody sequence is referred to herein as a humanized antibody. One type of chimeric antibody of the invention may have all or a portion of the V_{H} and/or a V_{L} domains from a rabbit anti-endosialin antibody (or from two or more rabbit anti-endosialin antibodies) and all or a portion of the constant region from a non-human primate antibody or from a human antibody.

A “human antibody” or “human sequence antibody” as used herein, means an antibody in which the variable and constant domain sequences are human sequences. The term encompasses antibodies with sequences encoded by human Ig genes or segments, but that have somatic mutations compared to the human germline sequence, or that may been changed, e.g. to decrease possible immunogenicity, increase affinity, eliminate cysteines that might cause undesirable folding, etc. The term encompasses such antibodies produced recombinantly in non-human cells, which might impart glycosylation not typical of human cells. The invention also encompasses an antigen-binding portion of an anti-endosialin antibody wherein the portion comprises rabbit and human sequences.

A particular type of chimeric antibody of the invention that is a humanized antibody is a human sequence antibody (the recipient antibody) or a fragment of such an antibody (e.g., Fv, Fab, Fab′, F(ab′)₂ or other antigen-binding subsequences) in which at least one CDR3 sequence has been replaced with a CDR3 sequence from a donor rabbit anti-endosialin antibody of the invention. In cases where the humanized antibody comprises a constant region, it is a human constant region. See, e.g., Riechmann et al. Nature, 332:323-327 (1988); U.S. Pat. Nos. 6,054,297; 5,821,337; 5,770,196; 5,766,886; 5,821,123; 5,869,619; 6,180,377; 6,013,256; 5,693,761; and 6,180,370.

Such an antibody may comprise a rabbit heavy chain CDR3 (HCDR3), a rabbit light chain CDR3 (LCDR3) or both. In some embodiments, the antibody contains six CDR regions from a rabbit anti-endosialin antibody. In any such humanized antibodies, the CDRs all may be from the same rabbit anti-endosialin antibody or CDRs from more than one rabbit anti-endosialin antibody may be combined in the chimeric antibody. For instance, a chimeric antibody may comprise a CDR1 from the light chain of a first rabbit anti-endosialin antibody, a CDR2 from the light chain of a second rabbit anti-endosialin antibody and a CDR3 from the light chain of a third rabbit anti-endosialin antibody, and CDRs from the heavy chain may be derived from one or more other anti-endosialin antibodies. Similarly, the human framework regions may be derived from a single human antibody or from two or more different human antibodies. One or more human (recipient) Fv framework region (FR) residues may sometimes be replaced by the residue(s) at the corresponding position in the non-human (donor) framework to, e.g., improve binding. Techniques for making such “back mutations” are well known to those of skill in the art. See, e.g., Queen et al. (1989) Proc. Nat. Acad. Sci. USA 86:10029; Co et al. 1991. Proc. Nat. Acad. Sci. USA 88:2869-2873; WO 90/07861; Tempest 1991. Biotechnology 9: 266-271. A humanized antibody of the invention may also comprise residues that are not found in the recipient antibody or in any imported CDR or FR sequences as a result of modifications that are made to further refine and optimize antibody performance. Methods for making humanized antibodies are well known to those of skill in the art of antibodies. See, e.g., European Patent No. 239 400; Jones et al. (1986) Nature 321:522-525; Riechmann et al. (1988) Nature 332:323-327; Verhoeyen et al. (1988) Science 239:1534-1536; Queen et al. (1989) Proc. Nat. Acad. Sci. USA 86:10029; Orlandi et al. (1989) Proc. Natl. Acad. Sci. USA 86:3833; U.S. Pat. No. 6,180,370, and European Patent No. 519 596, which describes antibody veneering of surface residues.

In one aspect, the invention provides rabbit anti-human endosialin monoclonal antibody 1-3-1 produced by the hybridoma cell line 1-3-1 (ATCC Deposit No. PTA-9653); 1-25-2 produced by the hybridoma cell line 1-25-2 (ATCC Deposit No. PTA-9650); 1-55-2 produced by the hybridoma cell line 1-55-2 (ATCC Deposit No. PTA-9652); and rabbit anti-mouse endosialin monoclonal antibody clone 8 produced by the hybridoma cell line clone 8 (ATCC Deposit No. PTA-9651). The antibodies were deposited with the American Type Culture Collection (ATCC), P.O. Box 1549, Manassas, Va. 20108, USA on Dec. 2, 2008, in accordance with the Budapest Treaty. As used herein, an antibody that is referred to by number is the same as a monoclonal antibody that is obtained from the hybridoma of the same number. For example, monoclonal antibody 1-3-1 is the same antibody as one obtained from hybridoma 1-3-1, or a subclone thereof. In another aspect, the invention provides humanized monoclonal antibodies Hu-1-3-1, Hu 1-25-2 and Hu 1-55-2, which comprise CDR sequences from rabbit monoclonal antibodies 1-3-1, 1-25-2 and 1-55-2, respectively, flanked by framework sequences from a human antibody and further comprising a human sequence constant region.

The invention encompasses an antibody that specifically binds endosialin wherein the amino acid sequence of the heavy chain comprises the amino acid sequence of the HCDR3, all three HCDRs, a portion of or the entire V_{H} or the full-length heavy chain of any of antibodies 1-3-1, 1-25-2, 1-55-2, clone 8, Hu 1-3-1, Hu 1-25-2 or Hu 1-55-2, the light chain amino acid sequence comprises the amino acid sequence of the LCDR3, all three LCDRs, a portion of or the entire V_{L} or the full-length light chain of any of antibodies 1-3-1, 1-25-2, 1-55-2, clone 8, Hu 1-3-1, Hu 1-25-2 or Hu 1-55-2, or both. The invention further encompasses an antigen-binding portion of such antibody. In embodiments comprising heavy chain components and light chain components from the specifically provided antibodies, the heavy chain components may be from the same or different source antibodies as the light chain components. By way of illustration, the invention encompasses an antibody that specifically binds endosialin and comprises one or more heavy chain components from antibody Hu 1-3-1 and one or more light chain components from antibody Hu 1-25-2. All possible combinations of heavy and light chain components are specifically encompassed to the same extent as if each individual combination were written out.

The invention further encompasses an anti-endosialin antibody that comprises a heavy chain CDR3 (HCDR3) amino acid sequence comprising the HCDR3 amino acid sequence of any one of antibodies 1-3-1 (SEQ ID NO:20); 1-25-2 (SEQ ID NO:52); 1-55-2 (SEQ ID NO:84), Hu 1-3-1 (SEQ ID NO:163), Hu 1-25-2 (SEQ ID NO: 206) or Hu 1-55-2 (SEQ ID NO: 238); a light chain amino acid sequence that comprises the light chain CDR3 (LCDR3) amino acid sequence comprising the LCDR3 amino acid sequence of any one of antibodies 1-3-1 (SEQ ID NO:36); 1-25-2 (SEQ ID NO:68); 1-55-2 (SEQ ID NO:100); Hu 1-3-1 (SEQ ID NO:181), Hu 1-25-2 (SEQ ID NO:222) or Hu 1-55-2 (SEQ ID NO:254), or an above-recited HCDR3 amino acid sequence and an above-recited LCDR3 amino acid sequence. In the last case, the heavy chain CDR3 amino acid sequence and the light chain CDR3 amino acid sequence may be from the same or from different antibodies. The invention contemplates all possible pairs of HCDR3 and LCDR3 sequences to the same extent as if all of the combinations were written out.

More specifically, the invention encompasses an anti-endosialin antibody that comprises:

(a) a heavy chain CDR3 amino acid sequence comprising the HCDR3 amino acid sequence of antibody 1-3-1 (SEQ ID NO:20) and a light chain CDR3 amino acid sequence comprising the LCDR3 amino acid sequence of antibody 1-3-1 (SEQ ID NO:36);

(b) a heavy chain CDR3 amino acid sequence comprising the HCDR3 amino acid sequence of antibody 1-25-2 (SEQ ID NO:52) and a light chain CDR3 amino acid sequence comprising the LCDR3 amino acid sequence of antibody 1-25-2 (SEQ ID NO:68);

(c) a heavy chain CDR3 amino acid sequence comprising the HCDR3 amino acid sequence of antibody 1-55-2 (SEQ ID NO:84) and a light chain CDR3 amino acid sequence comprising the LCDR3 amino acid sequence of antibody 1-55-2 (SEQ ID NO:100);

(d) a heavy chain CDR3 amino acid sequence comprising the HCDR3 amino acid sequence of antibody clone 8 (SEQ ID NO:116) and a light chain CDR3 amino acid sequence comprising the LCDR3 amino acid sequence of antibody clone 8 (SEQ ID NO:132);

(e) a heavy chain CDR3 amino acid sequence comprising the HCDR3 amino acid sequence of Hu 1-3-1 (SEQ ID NO:163) and a light chain CDR3 amino acid sequence comprising the LCDR3 amino acid sequence of Hu 1-3-1 (SEQ ID NO:181);

(f) a heavy chain CDR3 amino acid sequence comprising the HCDR3 amino acid sequence of antibody Hu 1-25-2 (SEQ ID NO:206) and a light chain CDR3 amino acid sequence comprising the LCDR3 amino acid sequence of antibody Hu 1-25-2 (SEQ ID NO:222); or

(g) a heavy chain CDR3 amino acid sequence comprising the HCDR3 amino acid sequence of antibody Hu 1-55-2 (SEQ ID NO:238) and a light chain CDR3 amino acid sequence comprising the LCDR3 amino acid sequence of antibody Hu 1-55-2 (SEQ ID NO:254).

Also encompassed by the invention are antibodies that specifically bind endosialin and comprise heavy chain CDR1, CDR2 and CDR3 amino acid sequences that are independently selected from the heavy chain CDR1, CDR2 and CDR3 amino acid sequences, respectively, of antibodies 1-3-1, 1-25-2, 1-55-2, Hu 1-3-1, Hu 1-25-2 or Hu 1-55-2, or such CDR sequences any of which may comprise less than 4 or less than 3 conservative amino acid substitutions and/or a total of three or fewer non-conservative amino acid substitutions.

By way of illustration, an anti-endosialin antibody of the invention may comprise a 1-3-1 heavy chain CDR1 amino acid sequence (SEQ ID NO: 16), a 1-25-2 heavy chain CDR2 amino acid sequence (SEQ ID NO: 50) and a 1-55-2 heavy chain CDR3 amino acid sequence (SEQ ID NO: 84). The invention contemplates binding molecules including antibodies that comprise any possible combination of provided HCDR1, HCDR2 and HCDR3 amino acid sequences to the same extent as if all such combinations were written out.

Similarly, the invention encompasses an anti-endosialin antibody light chain that comprises LCDR1, LCDR2 and LCDR3 amino acid sequences independently selected from the LCDR1, LCDR2 and LCDR3 amino acid sequences, respectively, of antibodies 1-3-1, 1-25-2, 1-55-2, Hu 1-3-1, Hu 1-25-2 or Hu 1-55-2, or such CDR sequences any of which may comprise less than 4 or less than 3 conservative amino acid substitutions and/or a total of three or fewer non-conservative amino acid substitutions, and antigen-binding portions of such antibodies. The invention contemplates all possible combinations of LCDR1, LCDR2 and LCDR3 to the same extent as if each combination were written out and further contemplates all possible combinations of HCDR sequences and LCDR sequences to the same extent as if the individual combinations were written out.

The invention further provides an antibody that specifically binds endosialin and comprises heavy chain CDR1, CDR2, and CDR3 amino acid sequences selected from: SEQ ID NOS: 16, 18, and 20, respectively; SEQ ID NOS: 48, 50, and 52, respectively; SEQ ID NOS: 80, 82, and 84, respectively; SEQ ID NOS: 112, 114, and 116, respectively; SEQ ID NOS: 159, 161, and 163, respectively, SEQ ID NOS: 202, 204 and 206, respectively; or SEQ ID NOS: 234, 236 and 238, respectively; or an antigen-binding portion of such an antibody.

Likewise, the invention provides an anti-endosialin antibody that comprises light chain CDR1, CDR2, and CDR3 amino acid sequences selected from: SEQ ID NOS: 32, 34, and 36, respectively; SEQ ID NOS: 64, 66, and 68, respectively; SEQ ID NOS: 96, 98, and 100, respectively; SEQ ID NOS: 128, 130 and 132, respectively; SEQ ID NOS: 177, 179 and 181, respectively; SEQ ID NOS: 218, 220 and 222, respectively; or SEQ ID NOS: 250, 252 and 254, respectively; or an antigen-binding portion of such an antibody.

The invention further encompasses an antibody that specifically binds endosialin and comprises heavy chain CDR1, CDR2, and CDR3 amino acid sequences (also referred to as an HCDR set) selected from: SEQ ID NOS: 16, 18, and 20, respectively; SEQ ID NOS: 48, 50, and 52, respectively; SEQ ID NOS: 80, 82, and 84, respectively; SEQ ID NOS: 159, 161, and 163, respectively; SEQ ID NOS: 202, 204 and 206, respectively; or SEQ ID NOS: 234, 236 and 238, respectively; and further comprises light chain CDR1, CDR2, and CDR3 amino acid sequences (also referred to as an LCDR set) selected from: SEQ ID NOS: 32, 34, and 36, respectively; SEQ ID NOS: 64, 66, and 68, respectively; SEQ ID NOS: 96, 98, and 100, respectively; or, SEQ ID NOS: 177, 179 and 181, respectively; SEQ ID NOS: 218, 220 and 222, respectively; or SEQ ID NOS: 250, 252 and 254, respectively; or an antigen-binding portion of such an antibody. The invention contemplates antibodies comprising any above-listed HCDR set paired with any above-listed LCDR set to the same extent as if each individual pair of CDR sets were written out.

Also within the invention is an anti-endosialin antibody comprising:

(a) heavy chain CDR1, CDR2, and CDR3 amino acid sequences of SEQ ID NOS: 16, 18 and 20, respectively, and light chain CDR1, CDR2 and CDR3 amino acid sequences of SEQ ID NOS: 32, 34 and 36, respectively;

(b) heavy chain CDR1, CDR2, and CDR3 amino acid sequences of SEQ ID NOS: 48, 50 and 52, respectively, and light chain CDR1, CDR2 and CDR3 amino acid sequences of SEQ ID NOS: 64, 66 and 68, respectively;

(c) heavy chain CDR1, CDR2, and CDR3 amino acid sequences of SEQ ID NOS: 80, 82 and 84, respectively, and light chain CDR1, CDR2 and CDR3 amino acid sequences of SEQ ID NOS: 96, 98 and 100, respectively;

(d) heavy chain CDR1, CDR2, and CDR3 amino acid sequences of SEQ ID NOS: 112, 114 and 116, respectively, and light chain CDR1, CDR2 and CDR3 amino acid sequences of SEQ ID NOS: 128, 130 and 132, respectively;

(e) heavy chain CDR1, CDR2, and CDR3 amino acid sequences of SEQ ID NOS: 159, 161 and 163, respectively, and light chain CDR1, CDR2 and CDR3 amino acid sequences of SEQ ID NOS: 177, 179 and 181, respectively;

(f) heavy chain CDR1, CDR2, and CDR3 amino acid sequences of SEQ ID NOS: 202, 204 and 206, respectively, and light chain CDR1, CDR2 and CDR3 amino acid sequences of SEQ ID NOS: 218, 220 and 222, respectively; or

(g) heavy chain CDR1, CDR2, and CDR3 amino acid sequences of SEQ ID NOS: 234, 236 and 238, respectively, and light chain CDR1, CDR2 and CDR3 amino acid sequences of SEQ ID NOS: 250, 252 and 254, respectively; or an antigen-binding portion of such an antibody.

The invention also provides an anti-endosialin antibody that comprises the heavy chain variable domain (V_{H}) amino acid sequence of antibody 1-3-1 (SEQ ID NO:14); 1-25-2 (SEQ ID NO:46); 1-55-2 (SEQ ID NO:78); clone 8 (SEQ ID NO:110); Hu 1-3-1 (SEQ ID NO:157); Hu 1-25-2 (SEQ ID NO:200) or Hu 1-55-2 (SEQ ID NO:232) or comprises a V_{H} amino acid sequence that is at least 80%, 85%, 90%, 95%, or 98% identical to one of said sequences, or an antigen binding portion of such antibody.

Likewise, the invention provides an anti-endosialin antibody that comprises the variable domain (V_{L}) amino acid sequence of antibody 1-3-1 (SEQ ID NO:30); 1-25-2 (SEQ ID NO:62); 1-55-2 (SEQ ID NO:94); clone 8 (SEQ ID NO:126); Hu 1-3-1 (SEQ ID NO:175); Hu 1-25-2 (SEQ ID NO:216) or Hu 1-55-2 (SEQ ID NO:248), or comprises a V_{L} amino acid sequence that is at least 80%, 85%, 90%, 95%, or 98% identical to one of said sequences, or an antigen binding portion of such antibody.

Still further, the invention provides an antibody that specifically binds endosialin and comprises a V_{H} amino acid sequence selected from SEQ ID NOs: 14, 46, 78, 157, 200 or 232, or a V_{H} amino acid sequence that is at least 80%, 85%, 90%, 95%, or 98% identical to one of said sequences, and further comprises a V_{L} amino acid sequence selected from SEQ ID NOS: 30, 62, 94, 175, 216 or 248, or a V_{L} amino acid sequence that is at least 80%, 85%, 90%, 95%, or 98% identical to one of said sequences, or an antigen binding portion of such antibody. The invention contemplates antibodies comprising any above-listed V_{H} sequence paired with any above-listed V_{L} sequence to the same extent as if each individual pair of V_{H} and V_{L} were written out.

In particular embodiments, the invention provides an anti-endosialin antibody comprising:

(a) a heavy chain variable domain comprising the sequence of SEQ ID NO:14 or a sequence that is at least 80%, 85%, 90%, 95%, or 98% identical to said sequence, and a light chain variable domain comprising the sequence of SEQ ID NO:30, or a sequence that is at least 80%, 85%, 90%, 95%, or 98% identical to said sequence;

(b) a heavy chain variable domain comprising the sequence of SEQ ID NO: 46 or a sequence that is at least 80%, 85%, 90%, 95%, or 98% identical to said sequence, and a light chain variable domain comprising the sequence of SEQ ID NO: 62, or a sequence that is at least 80%, 85%, 90%, 95%, or 98% identical to said sequence;

(c) a heavy chain variable domain comprising the sequence of SEQ ID NO:78 or a sequence that is at least 80%, 85%, 90%, 95%, or 98% identical to said sequence, and a light chain variable domain comprising the sequence of SEQ ID NO:94, or a sequence that is at least 80%, 85%, 90%, 95%, or 98% identical to said sequence;

(d) a heavy chain variable domain comprising the sequence of SEQ ID NO:110 or a sequence that is at least 80%, 85%, 90%, 95%, or 98% identical to said sequence, and a light chain variable domain comprising the sequence of SEQ ID NO: 126, or a sequence that is at least 80%, 85%, 90%, 95%, or 98% identical to said sequence;

(e) a heavy chain variable domain comprising the sequence of SEQ ID NO:157 or a sequence that is at least 80%, 85%, 90%, 95%, or 98% identical to said sequence, and a light chain variable domain comprising the sequence of SEQ ID NO:175, or a sequence that is at least 80%, 85%, 90%, 95%, or 98% identical to said sequence, or an antigen-binding portion of said antibody;

(f) a heavy chain variable domain comprising the sequence of SEQ ID NO:200 or a sequence that is at least 80%, 85%, 90%, 95%, or 98% identical to said sequence, and a light chain variable domain comprising the sequence of SEQ ID NO:216, or a sequence that is at least 80%, 85%, 90%, 95%, or 98% identical to said sequence, or an antigen-binding portion of said antibody; or

(g) a heavy chain variable domain comprising the sequence of SEQ ID NO:232 or a sequence that is at least 80%, 85%, 90%, 95%, or 98% identical to said sequence, and a light chain variable domain comprising the sequence of SEQ ID NO:248, or a sequence that is at least 80%, 85%, 90%, 95%, or 98% identical to said sequence, or an antigen-binding portion of said antibody.

Still further, the invention contemplates an antibody that specifically binds endosialin and that comprises the heavy chain amino acid sequence of SEQ ID NO: 156, with or without a signal sequence, or an amino acid sequence that is at least 80%, 85%, 90%, 95%, 96%, 97%, 98% or 99% identical to said sequence, the light chain amino acid sequence of SEQ ID NO: 174, with or without a signal sequence, or an amino acid sequence that is at least 80%, 85%, 90%, 95%, 96%, 97%, 98% or 99% identical to said sequence, or both; or an antigen-binding portion of said antibody.

The invention contemplates an antibody that specifically binds endosialin and that comprises the heavy chain amino acid sequence of SEQ ID NO: 185, with or without a signal sequence, or an amino acid sequence that is at least 80%, 85%, 90%, 95%, 96%, 97%, 98% or 99% identical to said sequence, the light chain amino acid sequence of SEQ ID NO: 187, with or without a signal sequence, or an amino acid sequence that is at least 80%, 85%, 90%, 95%, 96%, 97%, 98% or 99% identical to said sequence, or both; or an antigen-binding portion of said antibody.

The invention also contemplates an antibody that specifically binds endosialin and that comprises the heavy chain amino acid sequence of SEQ ID NO: 189, with or without a signal sequence, or an amino acid sequence that is at least 80%, 85%, 90%, 95%, 96%, 97%, 98% or 99% identical to said sequence, the light chain amino acid sequence of SEQ ID NO: 191, with or without a signal sequence, or an amino acid sequence that is at least 80%, 85%, 90%, 95%, 96%, 97%, 98% or 99% identical to said sequence, or both; or an antigen-binding portion of said antibody.

As applied to polypeptides, the term “substantial identity” means that two peptide sequences, when optimally aligned, such as by the programs GAP or BESTFIT using default gap weights as supplied with the programs, share at least 70%, 75% or 80% sequence identity, at least 90% or 95% sequence identity, and at least 97%, 98% or 99% sequence identity. In certain embodiments, residue positions that are not identical differ by conservative amino acid substitutions.

A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity may be adjusted upwards to correct for the conservative nature of the substitution. Means for making this adjustment are well-known to those of skill in the art. See, e.g., Pearson, Methods Mol. Biol. 243:307-31 (1994). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992), incorporated herein by reference. A “moderately conservative” replacement is any change having a nonnegative value in the PAM250 log-likelihood matrix.

Sequence identity for polypeptides is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters as specified by the programs to determine sequence homology or sequence identity between closely related polypeptides, such as homologous polypeptides from different species of organisms or between a wild type protein and a mutein thereof. See, e.g., GCG Version 6.1 (University of Wisconsin, WI). Polypeptide sequences also can be compared using FASTA using default or recommended parameters, see GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters as supplied with the programs. See, e.g., Altschul et al., J. Mol. Biol. 215:403-410 (1990); Altschul et al., Nucleic Acids Res. 25:3389-402 (1997).

One type of amino acid substitution that may be made is to change one or more cysteines in the antibody, which may be chemically reactive, to another residue, such as, without limitation, alanine or serine. In one embodiment, there is a substitution of a non-canonical cysteine. The substitution can be made in a CDR or framework region of a variable domain or in the constant domain of an antibody. In some embodiments, the cysteine is canonical.

Another type of amino acid substitution that may be made is to change any potential proteolytic sites in the antibody. Such sites may occur in a CDR or framework region of a variable domain or in the constant domain of an antibody. Substitution of cysteine residues and removal of proteolytic sites may decrease the risk of any heterogeneity in the antibody product and thus increase its homogeneity. Another type of amino acid substitution is to eliminate asparagine-glycine pairs, which form potential deamidation sites, by altering one or both of the residues. In some embodiments, amino acid substitution are used to insert or remove a glycosylation site. In some embodiments, the C-terminal lysine of the heavy chain of the anti-endosialin antibody of the invention may be proteolytically cleaved. In various embodiments of the invention, the heavy and light chains of the anti-endosialin antibodies may optionally include a signal sequence.

The invention further provides an antibody (including an anti-endosialin antibody) that comprises one or more of the heavy chain framework region (i.e., HFW1, HFW2, HFW3, and HFW4) sequences or one or more of the light chain framework region (i.e., LFW1, LFW2, LFW3, and LFW4) found in antibody 1-3-1, 1-25-2, 1-55-2 or clone 8 and listed in Table 1. Also encompassed by the invention is an antibody that comprises the one or more heavy chain framework sequences and one or more light chain framework sequences of antibody 1-3-1, 1-25-2 or 1-55-2. In some embodiments the heavy chain and light chain FW sequences are from the same reference antibody. The invention contemplates, however, all possible combinations of heavy chain FW sequences with light chain FW sequences to the same extent as if the individual combinations were written out.

A rabbit anti-endosialin antibody of the invention that comprises a heavy chain constant region can be of any isotype, i.e., IgG, IgA (including any sub-isotype, for example IgA1, IgA2, IgA3, IgA4, IgA5, IgA6, IgA7, IgA8, IgA9, IgA10, IgA11, IgA12 or IgA13), IgM or IgE. Similarly, a humanized antibody of the invention that comprises a heavy chain constant region can be of any human isotype or sub-isotype, i.e., IgG1, IgG2, IgG3, IgG4, an IgM, an IgE, an IgA1 or AgA2, or an IgD molecule. Some embodiments may be of a human IgG4 isotype (Ellison J. and Hood L. PNAS 79:1984-1988, 1982; Brusco A. et al., Eur J. Immunogenetics 25:349-355, 1998).

In the case of the light chain, embodiments comprising a light chain constant region, the light chain may be kappa or lambda light chains.

The class and subclass of anti-endosialin antibodies may be determined by any method known in the art. In general, the class and subclass of an antibody may be determined using antibodies that are specific for a particular class and subclass of antibody. Such antibodies are commercially available. The class and subclass can be determined by ELISA, or Western Blot or any other suitable techniques. Such techniques are known to those of skill in the art. Alternatively, the class and subclass may be determined by sequencing all or a portion of the constant domain of the heavy and/or light chains of the antibodies, comparing their amino acid sequences to the known amino acid sequences of various class and subclasses of immunoglobulins, and determining the class and subclass of the antibodies.

The invention encompasses a rabbit monoclonal antibody, or a humanized rabbit antibody or an antigen-binding portion of such an antibody that binds endosialin and competes or cross-competes with and/or binds the same epitope as: (a) an antibody selected from antibody 1-3-1; 1-25-2; 1-55-2; clone 8; Hu 1-3-1; Hu 1-25-2; or Hu 1-55-2; (b) an antibody that comprises a heavy chain variable domain having the amino acid sequence found in SEQ ID NO:14, SEQ ID NO:46, SEQ ID NO:78; SEQ ID NO:110; SEQ ID NO:157; SEQ ID NO:200; or SEQ ID NO:232; (c) an antibody that comprises a light chain variable domain having an amino acid sequence of the variable domain found in SEQ ID NO:30, SEQ ID NO:62, SEQ ID NO:94, SEQ ID NO:126; SEQ ID NO:175; SEQ ID NO:216; or SEQ ID NO:248; or (d) an antibody that comprises both a heavy chain variable domain as defined in (b) and a light chain variable domain as defined in (c). If two antibodies reciprocally compete with each other for binding to endosialin, they are said to cross-compete.

The term “epitope” includes any protein determinant capable of specific binding to an immunoglobulin or T-cell receptor or otherwise interacting with a molecule. Epitopic determinants generally consist of chemically-active surface groupings of molecules such as amino acids or carbohydrate or sugar side chains and generally have specific three dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another. An antibody is said to specifically bind an antigen when the dissociation constant is ≦1 mM, ≦100 nM or ≦10 nM. In certain embodiments, the K_{D} is 1 pM to 500 pM. In other embodiments, the K_{D} is between 500 pM to 1 μM, 1 μM to 100 nM or 100 mM to 10 nM. Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to endosialin, e.g., the antibodies compete for binding to the antigen. A high throughput process for “binning” antibodies based upon their cross-competition is described in International Patent Application No. WO 03/48731.

One can determine whether an antibody binds to the same epitope, competes or cross competes for binding with an anti-endosialin antibody provided herein by using methods known in the art. By way of illustration, one allows an anti-endosialin antibody exemplified herein (the reference antibody) to bind endosialin under saturating conditions and then measures the ability of a test antibody to bind endosialin. If the test antibody is able to bind endosialin at the same time as the reference anti-endosialin antibody, then the test antibody binds to a different epitope than the reference anti-endosialin antibody. However, if the test antibody is not able to bind endosialin at the same time, then the test antibody binds the same epitope, an overlapping epitope, or an epitope that is in close proximity to the epitope bound by the reference anti-endosialin antibody. To determine if a test antibody cross-competes with a reference antibody, the experiment is conducted reversing the antibodies, i.e., one allows the test antibody to bind endosialin and then measures the ability of the reference antibody to bind endosialin. These experiments can be performed using, e.g., ELISA, RIA, BIACORE®, or flow cytometry (FACS).

The endosialin binding molecules of the invention, including rabbit monoclonal antibody and humanized rabbit antibodies, that bind endosialin, may bind endosialin (or a portion such as the ECD) with a range of binding affinities, including high binding affinities. More particularly, an anti-endosialin binding molecule of the invention (e.g., an anti-endosialin antibody or an antigen-binding portion of such an antibody) may bind endosialin, or the ECD, of endosialin, with a K_{D} of about 2×10⁻⁷ M or less. The invention contemplates, thus, endosialin binding molecules that bind endosialin or endosialin ECD with a K_{D} of about 2×10⁻⁸ M, about 9×10⁻⁹ M, about 8×10⁻⁹ M, about 7×10⁻⁹ M, about 6×10⁻⁹ M, about 5×10⁻⁹ M, about 4×10⁻⁹ M, about 3×10⁻⁹ M, about 2×10⁻⁹ M, about 1×10⁻⁹ M, about 9×10⁻¹⁰ M, about 8×10⁻¹⁰ M about 7×10⁻¹⁰ M, about 6×10⁻¹⁰ M, about 5×10⁻¹⁰ M, about 4×10⁻¹⁰ M, about 3×10⁻¹⁰ M, about 2×10⁻¹⁰ M, about 1×10⁻¹⁰ M, about 9×10⁻¹¹ M, about 8×10⁻¹¹ M, about 7×10⁻¹¹ M, about 6×10⁻¹¹ M, about 5×10⁻¹¹ M, about 4×10⁻¹¹ M, about 3×10⁻¹¹ M, about 2×10⁻¹¹ M, about 1×10⁻¹¹M, about 9×10⁻¹² M, about 8×10⁻¹² M, about 7×10⁻¹² M, about 6×10⁻¹² M, about 5×10⁻¹² M, about 4×10⁻¹² M about 3×10⁻¹² M, about 2×10⁻¹² M, or about 1×10⁻¹² M, or less.

The invention further provides an endosialin binding molecule including a rabbit anti-endosialin monoclonal antibody or humanized rabbit antibody of the invention or an antigen-binding portion of the antibody, that binds to endosialin, or the ECD of endosialin, with substantially the same K_{D} as an antibody selected from antibody 1-3-1; 1-25-2; 1-55-2; clone 8; Hu 1-3-1; Hu 1-25-2; or Hu 1-55-2. More particularly, the binding to endosialin, or the ECD of endosialin, may be with substantially the same K_{D} as the binding of an antibody that comprises a heavy chain variable domain having the amino acid sequence of the V_{H} domain found in SEQ ID NO:14, SEQ ID NO:46, SEQ ID NO:78; SEQ ID NO:110; SEQ ID NO:157; SEQ ID NO:200; or SEQ ID NO:232, or that comprises the HCDR1, HCDR2 and HCDR3 amino acid sequences of a heavy chain variable domain having the amino acid sequence the V_{H} domain found in SEQ ID NO:14, SEQ ID NO:46, SEQ ID NO:78; SEQ ID NO:110, SEQ ID NO:157, SEQ ID NO: 200 or SEQ ID NO: 232. In still another embodiment, the antibody binds to endosialin, or the ECD of endosialin, with substantially the same K_{D} as an antibody that comprises a light chain variable domain having the amino acid sequence of the V_{L} domain found in SEQ ID NO:30, SEQ ID NO:62, SEQ ID NO:94, SEQ ID NO:126, SEQ ID NO:175, SEQ ID NO: 216 or SEQ ID NO: 248, or that comprises the LCDR1, LCDR2 and LCDR3 amino acid sequences of a light chain variable domain having the amino acid sequence the V_{L} domain found in SEQ ID NO:30, SEQ ID NO:62, SEQ ID NO:94, SEQ ID NO:126, SEQ ID NO:175, SEQ ID NO: 216 or SEQ ID NO: 248.

The term “K_{D}” refers to the equilibrium dissociation constant of a particular antibody-antigen interaction. One can determine whether an antibody has substantially the same K_{D} as an anti-endosialin antibody by using methods known in the art.

Still further, an endosialin binding molecule of the invention, including an anti-endosialin antibody or antigen-binding portion of such antibody includes those that have a low dissociation rate constant (k_{off}). In some embodiments, the anti-endosialin antibody may bind to endosialin, or the ECD of endosialin, with a k_{off} of 1.0×10⁻³ s⁻¹ or lower, a k_{off} of 5.0×10⁻⁴ s⁻¹ or lower or a k_{off} of 2×10⁻⁴ s⁻¹ or lower. In some embodiments, the k_{off} may be substantially the same as an antibody described herein, including an antibody selected from antibody 1-3-1; 1-25-2; 1-55-2; clone 8; Hu 1-3-1; Hu 1-25-2; or Hu 1-55-2. In some embodiments, the antibody may bind to endosialin, or the ECD of endosialin, with substantially the same k_{off} as an antibody that comprises (a) a heavy chain variable domain having the amino acid sequence of the V_{H} domain found in SEQ ID NO:14, SEQ ID NO:46, SEQ ID NO:78; SEQ ID NO:110; SEQ ID NO:157; SEQ ID NO:200; or SEQ ID NO:232, (b) a light chain variable domain having the amino acid sequence of the V_{L} domain found in SEQ ID NO:30, SEQ ID NO:62, SEQ ID NO:94, SEQ ID NO:126; SEQ ID NO:175; SEQ ID NO: 216; or SEQ ID NO:248, or (c) both (a) and (b). In still another embodiment, the antibody may bind to endosialin, or the ECD of endosialin, with substantially the same k_{off} as an antibody that comprises the HCDR set of a heavy chain variable domain having the amino acid sequence of the V_{H} domain found in SEQ ID NO:14, SEQ ID NO:46, SEQ ID NO:78; SEQ ID NO:110, SEQ ID NO:157, SEQ ID NO: 200 or SEQ ID NO: 232; the LCDR set of a light chain variable domain having the amino acid sequence of the V_{L} domain found in SEQ ID NO:30, SEQ ID NO:62, SEQ ID NO:94, SEQ ID NO:126, SEQ ID NO:175, SEQ ID NO: 216 or SEQ ID NO: 248; or both the HCDR set and the LCDR set, whether from the same of from different source antibodies.

The binding affinity and dissociation rate of an anti-endosialin antibody to endosialin can be determined by methods known in the art. The binding affinity can be measured by ELISAs, RIAs, flow cytometry, surface plasmon resonance, such as BIACORE®. The dissociation rate can be measured by surface plasmon resonance. The term “surface plasmon resonance”, as used herein, refers to an optical phenomenon that allows for the analysis of real-time biospecific interactions by detection of alterations in protein concentrations within a biosensor matrix, for example using the BIACORE® system (Pharmacia Biosensor AB, Uppsala, Sweden and Piscataway, N.J.). For further descriptions, see Jonsson U. et al., Ann. Biol. Clin. 51:19-26 (1993); Jonsson U. et al., Biotechniques 11:620-627 (1991); Jonsson B. et al., J. Mol. Recognit. 8:125-131 (1995); and Johnsson B. et al., Anal. Biochem. 198:268-277 (1991).

Some endosialin binding molecules of the invention, including anti-endosialin antibodies and antigen-binding portions of such antibodies, inhibit or reduce one or more activity of endosialin. The inhibition may be of the interaction of endosialin with an endosialin ligand. Endosialin ligands include extracellular matrix proteins, such as fibronectin, collagen (such as Collagen I or Collagen IV), and Mac-2 BP/90K (Tomkowicz et al. (2007) Proc. Natl. Acad. Sci. USA 104: 17965-17970 and Becker et al. (2008) FASEB Journal 22: 3059-3067, which are incorporated by reference in their entireties).

In particular, the invention provides rabbit and humanized rabbit anti-endosialin antibodies that reduce endosialin binding to fibronectin (see Example 7). In some cases, the antibody reduces binding of human endosialin to fibronectin with an IC₅₀ of about 20 μg/ml or less, about 25 μg/ml or less, about 50 μg/ml or less or about 75 μg/ml or less. In particular embodiments, antibodies 1-3-1, 1-25-2 and 1-55-2 inhibited endosialin binding to fibronectin with an IC₅₀ of 21.4 μg/ml, 41.5 μg/mL and 69.5 μg/mL, respectively. The IC₅₀ of an antagonist endosialin binding molecule of the invention, including a rabbit monoclonal antibody or a humanized rabbit monoclonal antibody that specifically binds endosialin or an antigen-binding portion of such an antibody, may be determined using any techniques known to those of skill in the art including competition assays and dose/response assays.

The inhibition of endosialin interaction with a ligand by an anti-endosialin antibody or an antigen-binding portion of the invention may inhibit pathways that are normally activated by this interaction. Endosialin interaction with endosialin ligands may promote cell migration, cell adhesion, and/or cell signaling. This activation of cell adhesion or cell migration may be mediated through integrins and/or matrix metalloproteinases (MMPs) (see U.S. Patent Application No. 2008/0248034, which is incorporated by reference in its entirety). Thus, in one embodiment, the inhibition of endosialin activity by an anti-endosialin antibody of the invention may inhibit or reduce the activation or expression of an integrin in the cell or in the tissue treated with the antibody. In another embodiment, the inhibition of endosialin activity by an anti-endosialin antibody of the invention may inhibit or reduce the activation or expression of one or more MMPs in the cell or in the tissue treated with the antibody. In preferred embodiments, the activation or the expression of MMP-1, MMP-2, MMP-8, MMP-9, MMP-12, MMP-13, or MMP-18 is inhibited or reduced by an antibody of the invention. Any assays known in the art for measuring the expression and activity of integrins and MMPs can be used for determining the inhibition by the anti-endosialin antibodies of the invention. For example, integrin activity assays and MMP zymography assays are described in Tomkowicz et al., supra. and U.S. Patent Application No. 2008/0248034. The inhibitory effects of an anti-endosialin antibody of the invention on cell adhesion to and cell migration through fibronectin and/or collagen may be determined by assays known in the art, such as the ones described in Tomkowicz et al., supra. and U.S. Patent Application No. 2008/0248034.

An anti-endosialin antibody or an antigen-binding portion may demonstrate both species and molecular selectivity. An anti-endosialin antibody of the invention may bind only to human endosialin, or may bind to endosialin from one or more non-human primate species. Some anti-endosialin antibodies of the invention bind to human and rabbit endosialin. In some embodiments, the anti-endosialin antibody does not bind to mouse, rat, or rabbit endosialin. Following the teachings of the specification, one may determine the species selectivity for the anti-endosialin antibody using methods well known in the art. For example, one may determine the species selectivity using Western blot, flow cytometry, ELISA, immunoprecipitation or RIA. One also may determine species specificity by assessing the ability of the antibody to inhibit endosialin functional responses (including cell adhesion, cell migration, integrin and MMP activity) using cells from that species.

An anti-endosialin antibody of the invention that comprises an Fc region may possess immune effector activity, such as antibody dependent (or Fc dependent) cellular cytotoxicity (ADCC/FcCC) activity. The ADCC activity may be mediated by Fc receptors on effector cells, which include but are not limited to cytotoxic T cells, natural killer (NK) cells, or macrophages, leading to cell lysis and/or death of the endosialin-expressing target cells. ADCC/FcCC activity of an anti-endosialin antibody of the invention may be measured using standard assays known in the art (see, e.g., U.S. Patent Application Publication No. 2006/0239911, which is incorporated by reference in its entirety). For example, an endosialin-expressing cell line may be exposed to various concentrations of an anti-endosialin antibody (or negative controls such as no antibody or control Ig) and activated effector cells, such as peripheral blood mononuclear cells (PBMCs). ADCC may be monitored by lactate dehydrogenase (LDH) release that occurs upon cell lysis of the endosialin-expressing cells. The activity of LDH may be measured by a spectrophotometric assay. An anti-endosialin antibody of the invention may elicit ADCC/FcCC activity that is at least 2-fold, at least 3-fold, at least 4-fold, at least 5-fold, at least 6-fold, at least 7-fold, at least 10-fold, at least 15-fold, at least 20-fold, at least 25-fold, at least 30-fold, or at least 50-fold higher than that occurring in the absence of antibody or with a control Ig. An anti-endosialin antibody of the invention may elicit this activity when used at concentrations such as at least 0.001 μg/ml, at least 0.01 μg/ml, at least 0.1 μg/ml, at least 1 μg/ml, or at least 5 μg/ml.

Further, an anti-endosialin antibody of the invention may possess complement dependent cytotoxicity (CDC). Antibodies that elicit such activity induce cell death of endosialin-expressing target cells through activation of the complement cascade. CDC activity of an anti-endosialin antibody of the invention may be measured using standard assays known in the art (see, e.g., U.S. Pat. No. 6,242,195 and Gazzano-Santoro et al. (1997) J Immunol Methods 202: 163-171, both of which are incorporated by reference in their entirety). For example, the assay may measure the relative number of viable cells by measuring the uptake and metabolism of ALAMARBLUE® (Invitrogen) dye. In brief, endosialin-expressing cells may be exposed to various concentrations of an anti-endosialin antibody (or negative controls such as no antibody or control Ig) and human complement. ALAMARBLUE® (Invitrogen) is then added to the cells and absorbance is measured after a period of incubation on a plate reader or fluorescence spectrophotometer or a UV-Vis spectrophotometer. Results may be expressed in relative fluorescence units (RFUs).

An anti-endosialin antibody of the invention may elicit CDC activity that is at least 2-fold, at least 3-fold, at least 4-fold, at least 5-fold, at least 6-fold, at least 7-fold, at least 10-fold, at least 15-fold, at least 20-fold, at least 25-fold, at least 30-fold, or at least 50-fold higher than that occurring in the absence of antibody or with a control Ig. An anti-endosialin antibody of the invention may elicit this activity when used at concentrations such as at least 0.001 μg/ml, at least 0.01 μg/ml, at least 0.1 μg/ml, at least 1 μg/ml, or at least 5 μg/ml.

According to the invention, an anti-endosialin antibody may internalize in a cell upon binding to endosialin on the cell surface. Such internalizing antibodies may be conjugated to chemotherapeutic agents, such as immunotoxins, radionuclides, or cytotoxic and cytostatic agents. Standard essays known in the art may be used to monitor internalization of an anti-endosialin antibody of the invention in endosialin-expressing cells (see, e.g., U.S. Patent Application Publication No. 2006/0239911, which is incorporated by reference in its entirety). For example, second immunotoxins, such as the Hum-ZAP assay (Advanced Targeting Systems, San Diego, Calif., USA), may be used to monitor internalization of anti-endosialin antibodies of the invention. Second immunotoxins are conjugates of a secondary antibody, such as a goat anti-human IgG, and the ribosome-inactivating protein, saporin. Such second immunotoxins may be selected so that they bind to an anti-endosialin antibody of the invention. If the anti-endosialin antibody is internalized, the saporin will inhibit protein synthesis and cause cell death. The cell viability of endosialin-expressing cells exposed to anti-endosialin antibodies of the invention and a second immunotoxin (or negative controls) may be measured with standard cell viability assays, such as those that read viable cell number by spectrophotometry. An anti-endosialin antibody of the invention may be considered to internalize if it reduces cell viability by at least 20%, at least 30%, at least 40%, at least 50%, at least 60%, at least 70%, at least 80%, or at least 90% in such an assay compared to a negative control.

A rabbit anti-endosialin antibody of the invention may be produced by any technique for obtaining a rabbit antibody to a desired antigen. For example, a rabbit anti-endosialin antibody of the invention may be produced by in vivo or in vitro immunization with an endosialin antigen. The endosialin antigen may be human endosialin (SEQ ID NOS: 1 and 2) or mouse endosialin (SEQ ID NOS: 3 and 4). Any suitable form of the endosialin antigen may be used, for example, full length endosialin, the extracellular domain (ECD) of endosialin, any fragment of the ECD sufficient to elicit an endosialin-specific antibody, any of the foregoing fused to an Fc region or cells expressing any of the foregoing.

According to the invention, the endosialin antigen may be administered with an adjuvant to stimulate the immune response. Any suitable adjuvant for use in rabbits or with rabbit cells may be used including complete or incomplete Freund's adjuvant, liposomes, ISCOMs (immunostimulating complexes), alum, mineral gels such as aluminum hydroxide and aluminum phosphate, lysolecithin, pluronic polyols, oil emulsions (oil-in-water, water-in-oil, etc), CpG, KLH, DNP and oil/surfactant based adjuvants such as RIBI® (muramyl dipeptides) and TITERMAX™. Other adjuvants that may protect the polypeptide from rapid dispersal by sequestering it in a local deposit, or that may contain substances that stimulate the host to secrete factors that are chemotactic for macrophages and other components of the immune system may be used according to the invention.

For in vivo immunization, rabbits may be immunized by any method known in the art. See, e.g., Harlow and Lane, Antibodies: A Laboratory Manual, New York: Cold Spring Harbor Press, 1990. Methods for immunizing non-human animals including rabbits are well known. If a polypeptide is being administered, the immunization schedule may involve two or more administrations of the polypeptide, spread out over several days or weeks. Examples 1 and 2 exemplify a method for producing anti-endosialin monoclonal antibodies in rabbits.

After immunization with an endosialin antigen, antibodies and/or antibody-producing cells may be obtained from the rabbit. Anti-endosialin antibody-containing serum may be obtained from the rabbit by bleeding or sacrificing the rabbit. The serum may be used as it is obtained from the rabbit, an immunoglobulin fraction may be obtained from the serum, or the anti-endosialin antibodies may be purified from the serum.

Also according to the invention, antibody producing cells (B lymphocytes) may be recovered from an immunized rabbit from peripheral blood, lymph nodes and/or the spleen.

The invention also encompasses production of rabbit anti-endosialin antibodies by any suitable method of in vitro immunization. See, e.g., United States Patent Application Publication 2006/0239911 which is incorporated herein by reference in its entirety for all purposes. Techniques using in vitro-primed splenocytes, “repertoire cloning” and in vitro immunization of peripheral blood mononuclear cells, e.g., are known. See, Boerner et al. (1991) J Immunol. 147: 86-95; Persson et al. (1991) Proc Nat Acad Sci USA 88: 2432-2436; Huang and Stollar (1991) J Immunol. Methods 141: 227-236; and Zafiropoulos et al. (1997) J Immunol. Methods 200: 181-190, all of which are incorporated by reference in their entirety.

Primary antibody-producing cells recovered from an immunized rabbit or in vitro immunized rabbit cells may be cultured. An antibody that specifically binds endosialin may be recovered from the cells and in the case of secreted antibodies, from culture medium. Techniques for recovering antibodies from antibody-producing cells are well-known in the art.

According to the invention, antibody-producing immortalized cell lines may be prepared from cells isolated from an immunized rabbit or from in vitro immunized cells. Any suitable method for immortalizing the antibody-producing cells may be used in the methods of the invention. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). In some cases, a rabbit cell producing an anti-endosialin antibody may be fused to a non-rabbit myeloma cell, such as a mouse myeloma cell to produce a heterohybridoma. Desirably, according to the methods of the invention, a rabbit cell producing an anti-endosialin antibody is fused to a rabbit fusion partner. See, for example, U.S. Pat. Nos. 5,675,063, 7,402,409 and 7,429,487, which are incorporated by reference in their entirety. Primary cells or immortalized cells may be screened for an antibody that specifically binds endosialin using any suitable technique including but not limited to enzyme-linked immunoassay (ELISA) or radioimmunoassay. Screening may utilize full-length endosialin, a portion thereof such as the ECD, or a cell expressing endosialin.

Anti-endosialin antibody-producing cells, e.g., hybridomas, may be selected, cloned and further screened for desirable characteristics, including robust growth, high antibody production and desirable antibody characteristics, as discussed further below. Antibody-producing cells can be expanded in vivo in syngeneic animals, in animals that lack an immune system, e.g., nude mice, or in cell culture in vitro. Methods of selecting, cloning and expanding hybridomas are well known to those of ordinary skill in the art.

Accordingly, in another aspect, the invention provides cells and cell lines (including hybridomas) that produce an anti-endosialin antibody, such as a rabbit or a humanized anti-endosialin antibody.

Also according to the invention, rabbit anti-endosialin antibodies may be selected and/or screened from a display library. For example, a rabbit anti-endosialin antibody of the invention may be isolated from phage in a phage display library prepared from nucleic acids obtained from in vitro or in vivo immunized rabbit cells. Kits for generating phage display libraries are commercially available (e.g., the Pharmacia Recombinant Phage Antibody System, catalog no. 27-9400-01; and the Stratagene SurfZAP phage display kit, catalog no. 240612). Clones from the library that produce antibodies with binding affinities of a desired magnitude for endosialin are identified and nucleic acids encoding the light and heavy chains or antigen-binding portions thereof are recovered and manipulated for standard recombinant expression. By way of example, one method for preparing the library of antibodies for use in phage display techniques comprises the steps of immunizing a rabbit with an endosialin antigen as described herein to create an immune response, extracting antibody-producing cells from the immunized rabbit; isolating RNA encoding heavy and light chains of antibodies of the invention from the extracted cells, reverse transcribing the RNA to produce cDNA, amplifying the cDNA using primers, and inserting the cDNA into a phage display vector such that antibodies are expressed on the phage.

Also within the invention are phage display libraries constructed using previously manipulated nucleotide sequences and screened in a similar fashion. For example, cDNAs encoding heavy and light chains may be independently supplied or linked to form a single chain Fv molecule (scFv) for production in the phage library. Also according to the invention, selection of rabbit anti-endosialin antibodies and antigen-binding portions of the invention may be accomplished in vitro using ribosome display (Hanes et al (1998) Proc Nat Acad Sci USA 95: 14130-14135), bacterial display (Samuelson et al (2002) J Biotechnology 96: 129-154) or yeast display (Kieke et al (1997) Protein Engineering 10: 1303-1310).

The resulting cells may be screened for immunoreactivity to endosialin. Techniques for identifying high affinity antibodies from such libraries are well known. See, e.g., Griffiths et al., (1994) EMBO J., 13:3245-3260; Nissim et al., (1994) EMBO J. 13:692-698 and by Griffiths et al., (1993) EMBO J. 12:725-734, all of which are incorporated by reference in their entirety.

An antibody that specifically binds endosialin or an antigen-binding portion of the invention may also be isolated from other types of display libraries. Methods and reagents that can be used in generating and screening antibody display libraries are known to those of skill in the art. See, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology 9:1370-1372 (1991); Hay et al., Hum. Antibod. Hybridomas 3:81-85 (1992); Huse et al., Science 246:1275-1281 (1989); McCafferty et al., Nature 348:552-554 (1990); Griffiths et al., EMBO J. 12:725-734 (1993); Hawkins et al., J. Mol. Biol. 226:889-896 (1992); Clackson et al., Nature 352:624-628 (1991); Gram et al., Proc. Natl. Acad. Sci. USA 89:3576-3580 (1992); Garrad et al., Bio/Technology 9:1373-1377 (1991); Hoogenboom et al., Nuc. Acid Res. 19:4133-4137 (1991); and Barbas et al., Proc. Natl. Acad. Sci. USA 88:7978-7982 (1991), all incorporated herein by reference.

To isolate and produce an anti-endosialin antibody with desired characteristics, a suitable anti-endosialin antibody is first used to select heavy and light chain sequences having similar binding activity toward endosialin, using the epitope imprinting methods described in PCT Publication No. WO 93/06213, incorporated herein by reference. The antibody libraries used in this method may be scFv libraries prepared and screened as described in PCT Publication No. WO 92/01047, McCafferty et al., Nature 348:552-554 (1990); and Griffiths et al., EMBO J. 12:725-734 (1993), all incorporated herein by reference.

Once initial V_{L} and V_{H} domains are selected, “mix and match” or “chain shuffling” experiments are performed, in which different pairs of the initially selected V_{L} and V_{H} segments are screened for endosialin binding to identify and recover preferred V_{L}/V_{H} pair combinations. Additionally, to further improve the quality of the antibody, the V_{L} and V_{H} segments of the preferred V_{L}/V_{H} pair(s) can be randomly mutated, preferably within the CDR3 region of V_{H} and/or V_{L}, in a process analogous to the in vivo somatic mutation process responsible for affinity maturation of antibodies during a natural immune response. This in vitro affinity maturation can be accomplished by amplifying V_{H} and V_{L} domains using PCR primers complementary to the V_{H} CDR3 or V_{L} CDR3, respectively, which primers have been “spiked” with a random mixture of the four nucleotide bases at certain positions such that the resultant PCR products encode V_{H} and V_{L} CDR3 segments into which random mutations have been introduced. These randomly mutated V_{H} and V_{L} segments can be re-screened for binding to endosialin (Wang et al., Nucleic Acids Res 30:e120 (2002), incorporated herein by reference).

Following screening and isolation of an anti-endosialin antibody of the invention from a recombinant immunoglobulin display library, nucleic acids encoding the selected antibody can be recovered from the display package (e.g., from the phage genome) and subcloned into other expression vectors by standard recombinant DNA techniques. If desired, the nucleic acid can further be manipulated to create other antibody forms of the invention, as described herein. To express a recombinant antibody isolated by screening of a combinatorial library, the DNA encoding the antibody is cloned into a recombinant expression vector and introduced into a mammalian host cells, as described herein.

In a further aspect, the present invention provides isolated nucleic acid molecules encoding a heavy chain, a V_{H} domain, a light chain, a V_{L} domain or a portion of a V_{H} or a V_{L} domain of an anti-endosialin antibody of the invention The nucleotide sequences encoding the heavy chain and light chain (or portions) may be on the same nucleic acid molecule or on separate nucleic acids.

The term “polynucleotide” as referred to herein means a polymeric form of nucleotides of at least 10 bases in length, either ribonucleotides or deoxynucleotides or a modified form of either type of nucleotide. The term includes single and double stranded forms.

The term “isolated polynucleotide” as used herein means a polynucleotide of genomic, cDNA, or synthetic origin or some combination thereof, which by virtue of its origin the “isolated polynucleotide” (1) is not associated with all or a portion of a polynucleotides with which the “isolated polynucleotide” is found in nature, (2) is operably linked to a polynucleotide to which it is not linked in nature, or (3) does not occur in nature as part of a larger sequence.

The term “naturally occurring nucleotides” as used herein includes deoxyribonucleotides and ribonucleotides. The term “modified nucleotides” as used herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al., Nucl. Acids Res. 14:9081 (1986); Stec et al., J. Am. Chem. Soc. 106:6077 (1984); Stein et al., Nucl. Acids Res. 16:3209 (1988); Zon et al., Anti-Cancer Drug Design 6:539 (1991); Zon et al., Oligonucleotides and Analogues: A Practical Approach, pp. 87-108 (F. Eckstein, Ed., Oxford University Press, Oxford England (1991)); U.S. Pat. No. 5,151,510; Uhlmann and Peyman, Chemical Reviews 90:543 (1990), the disclosures of which are hereby incorporated by reference. An oligonucleotide can include a label for detection, if desired.

The invention encompasses an isolated nucleic acid molecule comprising a nucleotide sequence that encodes at least a portion of the V_{H} amino acid sequence of an anti-endosialin antibody of the invention. The nucleic acid may comprise a nucleotide sequence that encodes a V_{H} domain of an antibody that specifically binds endosialin, wherein the nucleotide sequence encoding the V_{H} domain comprises a nucleotide sequence encoding:

(a) an HCDR3 amino acid sequence selected from the HCDR3 of antibody 1-3-1 (SEQ ID NO:20); 1-25-2 (SEQ ID NO:52); 1-55-2 (SEQ ID NO:84); clone 8 (SEQ ID NO:116); Hu 1-3-1 (SEQ ID NO:163), Hu 1-25-2 (SEQ ID NO: 206) or Hu 1-55-2 (SEQ ID NO: 238); or

(b) HCDR1, HCDR2 and HCDR3 amino acid sequences selected from the HCDR1, HCDR2 and HCDR3 amino acid sequences, respectively, of antibody 1-3-1 (SEQ ID NOS:16, 18 and 20, respectively); 1-25-2 (SEQ ID NOS:48, 50 and 52, respectively); 1-55-2 (SEQ ID NOS:80, 82 and 84, respectively); clone 8 (SEQ ID NOS:112, 114 and 116, respectively); Hu 1-3-1 (SEQ ID NOS:159, 161 and 163, respectively), Hu 1-25-2 (SEQ ID NOS: 202, 204 and 206, respectively), Hu 1-55-2 (SEQ ID NOS: 234, 236 and 238, respectively).

The invention also encompasses an isolated nucleic acid molecule comprising a nucleotide sequence that encodes a V_{H} domain amino acid sequence selected from the V_{H} amino acid sequence of antibody 1-3-1 (SEQ ID NO:14); 1-25-2 (SEQ ID NO:46); 1-55-2 (SEQ ID NO:78); clone 8 (SEQ ID NO:110); Hu 1-3-1 (SEQ ID NO:157); Hu 1-25-2 (SEQ ID NO:200); or Hu 1-55-2 (SEQ ID NO:232), a variant thereof, or said sequence having conservative amino acid mutations and/or a total of three or fewer non-conservative amino acid substitutions. In some embodiments, the nucleic acid molecule encodes a V_{H} amino acid sequence that is at least 70%, 75%, 80%, 85%, 90%, 95%, 97%, 98% or 99% identical to the V_{H} amino acid sequences of any one of V_{H} region of antibodies 1-3-1, 1-25-2, 1-55-2, clone 8, Hu 1-3-1, Hu 1-25-2 or Hu 1-55-2.

The term “percent sequence identity” in the context of nucleotide sequences means the residues in two sequences that are the same when aligned for maximum correspondence. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998); incorporated herein by reference). Unless otherwise specified, default parameters for a particular program or algorithm are used. For instance, percent sequence identity between nucleotide sequences can be determined using FASTA with its default parameters (a word size of 6 and the NOPAM factor for the scoring matrix) or using Gap with its default parameters as provided in GCG Version 6.1, incorporated herein by reference.

A reference to a nucleotide sequence encompasses its complement unless otherwise specified. Thus, a reference to a nucleic acid having a particular sequence should be understood to encompass its complementary strand, with its complementary sequence.

As used herein, the terms “percent sequence identity” and “percent sequence homology” are used interchangeably.

The term “substantial similarity” or “substantial sequence similarity,” when referring to a nucleic acid or fragment thereof, means that when optimally aligned with appropriate nucleotide insertions or deletions with another nucleic acid (or its complementary strand), there is nucleotide sequence identity in at least about 85%, at least about 90%, and at least about 95%, 96%, 97%, 98% or 99% of the nucleotide bases, as measured by any well-known algorithm of sequence identity, such as FASTA, BLAST or Gap, as discussed above.

The nucleic acid may comprise a nucleotide sequence that encodes a V_{H} domain of an antibody that specifically binds endosialin wherein the V_{H} nucleotide sequence comprises:

(a) the HCDR3 nucleotide sequence of an antibody selected from antibody 1-3-1 (SEQ ID NO:12); 1-25-2 (SEQ ID NO:44); 1-55-2 (SEQ ID NO:76); clone 8 (SEQ ID NO:108); Hu 1-3-1 (SEQ ID NO:154), Hu 1-25-2 (SEQ ID NO: 198) or Hu 1-55-2 (SEQ ID NO: 230);

(b) the HCDR1, HCDR2 and HCDR3 nucleotide sequences of an antibody selected from antibody 1-3-1 (SEQ ID NOS:8, 10 and 12, respectively); 1-25-2 (SEQ ID NOS:40, 42 and 44, respectively); 1-55-2 (SEQ ID NOS:72, 74 and 76, respectively); clone 8 (SEQ ID NOS:104, 106 and 108, respectively); Hu 1-3-1 (SEQ ID NOS:150, 152 and 154, respectively); Hu 1-25-2 (SEQ ID NOS: 194, 196 and 198, respectively) or Hu 1-55-2 (SEQ ID NOS: 226, 228 and 230, respectively); or

(c) the V_{H} domain nucleotide sequence of an antibody selected from antibody 1-3-1 (SEQ ID NO:6); 1-25-2 (SEQ ID NO:38); 1-55-2 (SEQ ID NO:70); clone 8 (SEQ ID NO:102); Hu 1-3-1 (SEQ ID NO:148); Hu 1-25-2 (SEQ ID NO:192); or Hu 1-55-2 (SEQ ID NO:224).

An isolated nucleic acid molecule of the invention also may comprise a nucleotide sequence that encodes a full-length heavy chain of an antibody selected from antibody 1-3-1, 1-25-2, 1-55-2, clone 8, Hu 1-3-1, Hu 1-25-2 or Hu 1-55-2, with or without a signal sequence, or a heavy chain comprising one or more mutations, as discussed herein. The nucleic acid may comprise a nucleotide sequence encoding SEQ ID NO: 156, with or without a signal sequence, and in particular, may comprise the nucleotide sequence of SEQ ID NO:147, with or without a signal sequence, may comprise a nucleotide sequence encoding SEQ ID NO: 185, with or without a signal sequence, in particular may comprise the nucleotide sequence of SEQ ID NO: 184, with or without a signal sequence, or may comprise a nucleotide sequence encoding SEQ ID NO: 189, with or without a signal sequence, and in particular, may comprise the nucleotide sequence of SEQ ID NO: 188, with or without a signal sequence.

The invention further provides an isolated nucleic acid molecule that encodes at least a portion of the V_{L} amino acid sequence of an anti-endosialin antibody of the invention. The nucleic acid may comprise a nucleotide sequence that encodes a V_{L} domain of an antibody that specifically binds endosialin, wherein the nucleotide sequence encoding the V_{L} domain comprises a nucleotide sequence encoding:

(a) an LCDR3 amino acid sequence selected from the LCDR3 of antibody 1-3-1 (SEQ ID NO:36); 1-25-2 (SEQ ID NO:68); 1-55-2 (SEQ ID NO:100); clone 8 (SEQ ID NO:132); Hu 1-3-1 (SEQ ID NO:181), Hu 1-25-2 (SEQ ID NO: 222), or Hu 1-55-2 (SEQ ID NO: 254); or

(b) LCDR1, LCDR2 and LCDR3 amino acid sequences selected from the HCDR1, HCDR2 and HCDR3 amino acid sequences, respectively, of antibody 1-3-1 (SEQ ID NOS:32, 34 and 36, respectively); 1-25-2 (SEQ ID NOS:64, 66 and 68, respectively); 1-55-2 (SEQ ID NOS:96, 98 and 100, respectively); clone 8 (SEQ ID NOS:128, 130 and 132, respectively); Hu 1-3-1 (SEQ ID NOS:177, 179 and 181, respectively), Hu 1-25-2 (SEQ ID NOS: 218, 220 and 222, respectively) or Hu 1-55-2 (SEQ ID NOS: 250, 252 and 254, respectively).

The invention also encompasses an isolated nucleic acid molecule comprising a nucleotide sequence that encodes a V_{L} domain amino acid sequence selected from the V_{L} amino acid sequence of antibody 1-3-1 (SEQ ID NO:30); 1-25-2 (SEQ ID NO:62); 1-55-2 (SEQ ID NO:94); clone 8 (SEQ ID NO:126); Hu 1-3-1 (SEQ ID NO:175), Hu 1-25-2 (SEQ ID NO: 216) or Hu 1-55-2 (SEQ ID NO: 248), a variant thereof, or said sequence having conservative amino acid mutations and/or a total of three or fewer non-conservative amino acid substitutions. In some embodiments, the nucleic acid molecule encodes a V_{L} amino acid sequence that is at least 70%, 75%, 80%, 85%, 90%, 95%, 97%, 98% or 99% identical to the V_{L} amino acid sequences of any one of V_{L} region of antibodies 1-3-1, 1-25-2, 1-55-2, clone 8, Hu 1-3-1, Hu 1-25-2, or Hu 1-55-2.

The nucleic acid may comprise a nucleotide sequence that encodes a V_{L} domain of an antibody that specifically binds endosialin wherein the V_{L} nucleotide sequence comprises:

(a) the LCDR3 nucleotide sequence of an antibody selected from antibody 1-3-1 (SEQ ID NO:28); 1-25-2 (SEQ ID NO:56); 1-55-2 (SEQ ID NO:88); clone 8 (SEQ ID NO:120); Hu 1-3-1 (SEQ ID NO:168), Hu 1-25-2 (SEQ ID NO: 214) or Hu 1-55-2 (SEQ ID NO: 246);

(b) the LCDR1, LCDR2 and LCDR3 nucleotide sequences of an antibody selected from antibody 1-3-1 (SEQ ID NOS:24, 26 and 28, respectively); 1-25-2 (SEQ ID NOS:56, 58 and 60, respectively); 1-55-2 (SEQ ID NOS:88, 90 and 92, respectively); clone 8 (SEQ ID NOS:120, 122 and 124, respectively); Hu 1-3-1 (SEQ ID NOS:168, 170 and 172, respectively); Hu 1-25-2 (SEQ ID NOS: 210, 212 and 214, respectively) or Hu 1-55-2 (SEQ ID NOS: 242, 244 and 246, respectively); or

(c) the V_{L} domain nucleotide sequence of an antibody selected from antibody 1-3-1 (SEQ ID NO:22); 1-25-2 (SEQ ID NO:54); 1-55-2 (SEQ ID NO:86); clone 8 (SEQ ID NO:118); Hu 1-3-1 (SEQ ID NO:166); Hu 1-25-2 (SEQ ID NO:208) or Hu 1-55-2 (SEQ ID NO:240).

A nucleic acid molecule of the invention also may comprise a nucleotide sequence that encodes a full-length light chain of an antibody selected from antibody 1-3-1, 1-25-2, 1-55-2, clone 8, Hu 1-3-1, Hu 1-25-2 or Hu 1-55-2, with or without a signal sequence, or a light chain comprising one or more mutations, as discussed herein. The nucleic acid may comprise a nucleotide sequence encoding SEQ ID NO: 174, with or without a signal sequence, and in particular, may comprise the nucleotide sequence of SEQ ID NO:165, with or without a signal sequence; may comprise a nucleotide sequence encoding SEQ ID NO: 187, with or without a signal sequence, and in particular, may comprise the nucleotide sequence of SEQ ID NO: 186, with or without a signal sequence; may comprise a nucleotide sequence encoding SEQ ID NO: 191, with or without a signal sequence, and in particular, may comprise the nucleotide sequence of SEQ ID NO: 190, with or without a signal sequence.

Also encompassed by the invention is an isolated nucleic acid molecule encoding an anti-endosialin binding molecule, including an antibody or an antigen-binding portion of an antibody, wherein the nucleic acid molecule comprises a nucleotide sequence that encodes:

(a) the HCDR3 amino acid sequence of SEQ ID NO: 20 and the LCDR3 amino acid sequence of SEQ ID NO: 36;

(b) the HCDR3 amino acid sequence of SEQ ID NO: 52 and the LCDR3 amino acid sequence of SEQ ID NO: 68;

(c) the HCDR3 amino acid sequence of SEQ ID NO: 84 and the LCDR3 amino acid sequence of SEQ ID NO: 100;

(d) the HCDR3 amino acid sequence of SEQ ID NO: 116 and the LCDR3 amino acid sequence of SEQ ID NO: 132;

(e) the HCDR3 amino acid sequence of SEQ ID NO: 163 and the LCDR3 amino acid sequence of SEQ ID NO: 181;

(f) the HCDR3 amino acid sequence of SEQ ID NO: 206 and the LCDR3 amino acid sequence of SEQ ID NO: 222; or

(g) the HCDR3 amino acid sequence of SEQ ID NO: 238 and the LCDR3 amino acid sequence of SEQ ID NO: 254.

In particular, the nucleic acid encoding the anti-endosialin binding molecule may comprise HCDR3 and LCDR3 nucleotide sequences selected from:

(a) SEQ ID NO: 12 and SEQ ID NO: 28, respectively;

(b) SEQ ID NO: 44 and SEQ ID NO: 60, respectively;

(c) SEQ ID NO: 76 and SEQ ID NO: 92, respectively;

(d) SEQ ID NO: 108 and SEQ ID NO: 124, respectively;

(e) SEQ ID NO: 154 and SEQ ID NO: 172, respectively;

(f) SEQ ID NO: 198 and SEQ ID NO: 214, respectively; or

(g) SEQ ID NO: 230 and SEQ ID NO: 246, respectively.

An isolated nucleic acid molecule of the invention encoding an anti-endosialin binding molecule, including an antibody or an antigen-biding portion of an antibody, also may be a nucleic acid molecule that comprises nucleotide sequences encoding:

(a) HCDR1, HCDR2 and HCDR3 amino acid sequences of SEQ ID NOS: 16, 18 and 20, respectively, and LCDR1, LCDR2 and LCDR3 amino acid sequences SEQ ID NOS: 32, 34 and 36, respectively;

(b) HCDR1, HCDR2 and HCDR3 amino acid sequences of SEQ ID NOS: 48, 50 and 52, respectively, and LCDR1, LCDR2 and LCDR3 amino acid sequences SEQ ID NOS:64, 66 and 68, respectively;

(c) HCDR1, HCDR2 and HCDR3 amino acid sequences of SEQ ID NOS: 80, 82 and 84, respectively, and LCDR1, LCDR2 and LCDR3 amino acid sequences SEQ ID NOS:96, 98 and 100, respectively;

(d) HCDR1, HCDR2 and HCDR3 amino acid sequences of SEQ ID NOS: 112, 114 and 116, respectively, and LCDR1, LCDR2 and LCDR3 amino acid sequences SEQ ID NOS:128, 130 and 132, respectively;

(e) HCDR1, HCDR2 and HCDR3 amino acid sequences of SEQ ID NOS: 159, 161 and 163, respectively, and LCDR1, LCDR2 and LCDR3 amino acid sequences SEQ ID NOS:177, 179 and 181, respectively;

(f) HCDR1, HCDR2 and HCDR3 amino acid sequences of SEQ ID NOS: 202, 204 and 206, respectively, and LCDR1, LCDR2 and LCDR3 amino acid sequences SEQ ID NOS: 218, 220 and 222, respectively; or

(g) HCDR1, HCDR2 and HCDR3 amino acid sequences of SEQ ID NOS: 234, 236 and 238, respectively, and LCDR1, LCDR2 and LCDR3 amino acid sequences SEQ ID NOS: 250, 252 and 254, respectively.

In particular, the nucleic acid encoding the anti-endosialin binding molecule may comprise HCDR1, HCDR2 and HCDR3 nucleotide sequences and LCDR1, LCDR2 and LCDR3 nucleotide sequences selected from:

(a) SEQ ID NOS: 8, 10 and 12, respectively, and SEQ ID NOS: 24, 26 and 28, respectively;

(b) SEQ ID NOS: 40, 42 and 44, respectively, and SEQ ID NOS: 56, 58 and 60, respectively;

(c) SEQ ID NOS: 72, 74 and 76, respectively, and SEQ ID NOS: 88, 90 and 92, respectively;

(d) SEQ ID NOS: 104, 106 and 108, respectively, and SEQ ID NOS: 120, 122 and 124, respectively;

(e) SEQ ID NOS: 150, 152 and 154, respectively, and SEQ ID NOS: 168, 170 and 172, respectively;

(f) SEQ ID NOS: 194, 196 and 198, respectively, and SEQ ID NOS: 210. 212 and 214, respectively; or

(g) SEQ ID NOS: 226, 228 and 230, respectively, and SEQ ID NOS: 242, 244 and 246, respectively.

An isolated nucleic acid molecule of the invention likewise may comprise nucleotide sequences encoding a V_{H} amino acid sequence and a V_{L} amino acid sequence selected from:

(a) the V_{H} amino acid sequence of SEQ ID NO: 14 and the V_{L} amino acid sequence of SEQ ID NO: 30, respectively;

(b) the V_{H} amino acid sequence of SEQ ID NO: 46 and the V_{L} amino acid sequence of SEQ ID NO: 62, respectively;

(c) the V_{H} amino acid sequence of SEQ ID NO: 78 and the V_{L} amino acid sequence of SEQ ID NO: 94, respectively;

(d) the V_{H} amino acid sequence of SEQ ID NO: 110 and the V_{L} amino acid sequence of SEQ ID NO: 126, respectively;

(e) the V_{H} amino acid sequence of SEQ ID NO: 157 and the V_{L} amino acid sequence of SEQ ID NO: 175, respectively;

(f) the V_{H} amino acid sequence of SEQ ID NO: 200 and the V_{L} amino acid sequence of SEQ ID NO: 216, respectively; or

(g) the V_{H} amino acid sequence of SEQ ID NO: 232 and the V_{L} amino acid sequence of SEQ ID NO: 248, respectively; or an antigen-binding portion of an above-listed V_{H} or V_{L}.

In particular, the nucleic acid may comprise a V_{H} nucleotide sequence and a V_{L} nucleotide sequence selected from:

(a) SEQ ID NO: 6 and SEQ ID NO: 22, respectively;

(b) SEQ ID NO: 38 and SEQ ID NO: 54, respectively;

(c) SEQ ID NO: 70 and SEQ ID NO: 86, respectively;

(d) SEQ ID NO: 102 and SEQ ID NO: 118, respectively;

(e) SEQ ID NO: 148 and SEQ ID NO: 166, respectively;

(f) SEQ ID NO: 192 and SEQ ID NO: 208, respectively; or

(g) SEQ ID NO: 224 and SEQ ID NO: 240, respectively.

Nucleic acids encoding the heavy or light chain of an anti-endosialin antibody or portions thereof can be isolated from any source that produces such an antibody. In various embodiments, the nucleic acid molecules are isolated from a B cell isolated from a rabbit immunized with endosialin or from an immortalized cell derived from such a B cell that expresses an anti-endosialin antibody. Methods of isolating mRNA encoding an antibody are well-known in the art. See, e.g., Sambrook et al., supra. The mRNA may be used to produce cDNA, e.g., for use in the polymerase chain reaction (PCR) or cDNA cloning of antibody genes.

In some embodiments, a nucleic acid encoding a heavy chain of an anti-endosialin antibody of the invention can comprise a nucleotide sequence encoding a V_{H} domain of the invention joined in-frame to a nucleotide sequence encoding a heavy chain constant domain from any source. Similarly, a nucleic acid molecule encoding a light chain of an anti-endosialin antibody of the invention can comprise a nucleotide sequence encoding a V_{L} domain of the invention joined in-frame to a nucleotide sequence encoding a light chain constant domain from any source. Accordingly, the invention encompasses a V_{H}, a V_{L}, or both of an anti-endosialin antibody joined in frame to a human constant region and nucleic acids encoding them.

A nucleic acid molecule encoding the variable domain of the heavy (V_{H}) and/or light (V_{L}) chains may be “converted” to a nucleic acid encoding a full-length antibody chain. For such a “conversion”, a nucleic acid molecule encoding the V_{H} or V_{L} domain is inserted into an expression vector already comprising a nucleic acid encoding a heavy chain constant (C_{H}) or light chain constant (C_{L}) domain, respectively, such that the V_{H} segment is operably linked to the C_{H} segment(s) within the vector, and/or the V_{L} segment is operably linked to the C_{L} segment within the vector. In another embodiment, a nucleic acid molecule encoding a V_{H} and/or V_{L} domain is converted into a full-length antibody gene by linking, e.g., ligating, the nucleic acid molecule encoding the V_{H} and/or V_{L} domains to a nucleic acid molecule encoding a C_{H} and/or C_{L} domain, respectively, using standard molecular biological techniques. Nucleotide sequences of human and rabbit heavy and light chain immunoglobulin constant domain genes are known in the art. See, e.g., Kabat et al., Sequences of Proteins of Immunological Interest, 5th Ed., NIH Publ. No. 91-3242, 1991 and Sohma et al. (1995) Proc. Nat. Acad. Sci. USA 92: 4937-4941, incorporated by reference in their entirety. One or more nucleic acid molecules encoding the full-length heavy and light chains may then be expressed from a cell into which they have been introduced and the anti-endosialin antibody isolated.

A nucleic acid of the invention may encode an amino acid substitution that improves a property of the antibody, for example, adding or removing a glycosylation site or encoding a substitutions that improves the stability or half-life of the antibody. The nucleic acid also may contain “silent” mutations to add or remove a restriction enzyme site, for example to facilitate cloning of the nucleic acid into a particular expression vector. Cysteine residues that are not necessary to maintain the conformation of the anti-endosialin antibodies and antigen-binding portions of the invention may be replaced (for example, with serine residues) to improve oxidative stability of the antibody or portion and prevent unnecessary crosslinking. Alternatively, cysteine residues may be added to the antibodies or portions of the invention to improve stability by the addition of cysteine bonds. In embodiments comprising heavy chain and/or light chain constant regions, the nucleotide sequence encoding the constant region may contain one or more mutations compared to a germline constant region sequence.

The nucleic acid molecules of the invention may be used to recombinantly express anti-endosialin antibodies. The nucleic acid molecules also may be used to produce chimeric antibodies, bispecific antibodies, single chain antibodies, immunoadhesins, diabodies, mutated antibodies and endosialin binding molecules comprising non-immunoglobulin scaffolds, as described herein. The rabbit nucleic acid molecules may be used for antibody humanization, also as described herein.

The invention in a further aspect provides vectors comprising any of the aforementioned nucleic acid molecules. The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “expression vectors”). The vectors may comprise nucleic acid molecules encoding a fusion protein, a modified antibody, antibody fragments, or the like.

In some embodiments, the anti-endosialin antibodies or antigen-binding portions of the invention are expressed by inserting DNAs encoding partial or full-length light and/or heavy chains, obtained as described herein, into expression vectors such that the genes are operably linked to necessary expression control sequences such as transcriptional and translational control sequences.

“Operably linked” sequences include both expression control sequences that are contiguous with the gene of interest and expression control sequences that act in trans or at a distance to control the gene of interest. The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

Expression vectors include plasmids, retroviruses, adenoviruses, adeno-associated viruses (AAV), plant viruses such as cauliflower mosaic virus or tobacco mosaic virus, cosmids, YACs, EBV derived episomes, and the like. In some instances, a nucleic acid encoding an antibody, an antibody chain or an antigen-binding portion of the invention is ligated into a vector such that transcriptional and translational control sequences within the vector serve their intended function of regulating the transcription and translation of the antibody gene. As is well-known to the skilled worker, the expression vector and expression control sequences are chosen to be compatible with the desired level of expression, the expression host cell used and the like. The nucleic acid encoding the antibody light chain or portion and the antibody heavy chain or portion can be inserted into separate vectors or into the same expression vector. The nucleic acids are inserted into the expression vector by standard methods (e.g., ligation of complementary restriction sites on the antibody gene fragment and vector, or blunt end ligation if no restriction sites are present).

In some cases, the vector is one that encodes a functionally complete C_{H} or C_{L} immunoglobulin sequence (which may be rabbit or human), with appropriate restriction sites engineered so that any V_{H} or V_{L} sequence can be inserted and expressed, as described herein. In such vectors, splicing usually occurs between the splice donor site in the inserted J region and the splice acceptor site preceding the human C domain, and also at the splice regions that occur within the human C_{H} exons. Polyadenylation and transcription termination occur at native chromosomal sites downstream of the coding regions. The recombinant expression vector also can encode a signal peptide that facilitates secretion of the antibody chain from a host cell. The nucleic acid encoding the antibody chain may be cloned into the vector such that the signal peptide is linked in-frame to the amino terminus of the immunoglobulin chain. The signal peptide can be an immunoglobulin signal peptide or a heterologous signal peptide (i.e., a signal peptide from a non-immunoglobulin protein).

In addition to the nucleic acid encoding the antibody chain or portion of the invention, the recombinant expression vectors of the invention carry regulatory sequences that control the expression of the antibody in a host cell. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615, incorporated herein by reference in their entirety. Methods for expressing antibodies in plants, including a description of promoters and vectors, as well as transformation of plants is known in the art. See, e.g., U.S. Pat. No. 6,517,529, incorporated herein by reference in its entirety. Methods of expressing polypeptides in bacterial cells, fungal cells, e.g., yeast cells, or insect cells infected with baculovirus, e.g., Spodoptera frugiperda cells, such as Sf9 or Sf21 cell lines, are also well known in the art.

The recombinant expression vectors of the invention also may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, incorporated herein by reference in their entirety). For example, typically the selectable marker gene confers resistance to drugs, such as G418, hygromycin, methotrexate or kanamycin, on a host cell into which the vector has been introduced. Selectable marker genes include the dihydrofolate reductase (DHFR) gene (for use in dhfr-host cells with methotrexate selection/amplification), the neo gene (for G418 selection), and the glutamate synthetase (GS) gene.

Nucleic acid molecules encoding endosialin binding molecules of the invention and vectors comprising these nucleic acid molecules can be used for transfection of a suitable mammalian, plant, bacterial, insect or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455, incorporated herein by reference in their entirety). Methods of transforming plant cells are well known in the art, including, e.g., Agrobacterium-mediated transformation, biolistic transformation, direct injection, electroporation and viral transformation. Methods of transforming bacterial, insect cells, and yeast cells are also well known in the art.

Mammalian cell lines available as hosts for expression are well known in the art and include many immortalized cell lines available from the American Type Culture Collection (ATCC). These include, inter alia, Chinese hamster ovary (CHO) cells, NS0 cells, SP2 cells, HEK-293T cells, NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 or Sf21 cells, Drosophila S2 cells, and Trichoplusia ni High Five™ cells (Invitrogen, Carlsbad, Calif., USA). When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Plant host cells include, e.g., Nicotiana, Arabidopsis, duckweed, corn, wheat, potato, etc. Bacterial host cells include E. coli and Streptomyces species. Yeast host cells include Schizosaccharomyces pombe, Saccharomyces cerevisiae and Pichia pastoris.

Further, expression from production cell lines can be enhanced using a number of known techniques. For example, the glutamine synthetase gene expression system (the GS system) is a common approach for enhancing expression under certain conditions. The GS system is discussed in whole or part in connection with European Patent Nos. 216 846, 256 055, 323 997 and 338 841, incorporated herein by reference in their entirety.

Host cells for producing an endosialin binding molecule of the invention such as an antibody that specifically binds endosialin or an antigen-binding portion of such an antibody, may be hypermutable cells, cell lines or transgenic non-human mammal cells, in which the endogenous mismatch repair (MMR) activity has been inhibited (see, e.g., U.S. Pat. Nos. 7,319,036, 7,235,643, 6,825,038, 6,808,894 and 6,146,894, which are incorporated herein by reference in their entirety). In one embodiment, the MMR activity may be inhibited by introduction of dominant negative alleles of MMR genes such as PMS1, PMS2, PMS2-134, PMSR2, PMSR3, MLH1, MLH2, MLH3, MLH4, MLH5, MLH6, PMSL9, MSH2 or MSH2 into a cell. In another embodiment, MMR may be inhibited using chemical inhibitors of mismatch repair (e.g., PCT Publication No. WO 02/54856, which is incorporated by reference in its entirety). Such methods of “directed evolution” may be used, for example, on the hybridomas producing the rabbit monoclonal anti-endosialin antibodies of the invention. These methods of inhibiting MMR activity can be used to generate cells with enhanced levels of anti-endosialin antibody production. Such cells, cell lines or transgenic animals also generate genetic variability in the anti-endosialin antibodies they produce and can be used to generate anti-endosialin antibodies with desired characteristics, such as increased affinity.

According to the invention, the endosialin binding molecules may be produced in bioreactors to facilitate large scale production.

The invention also contemplates nucleic acids (including ones that encode anti-endosialin antibodies or antigen-binding portions of such antibodies) that are synthesized in vitro and preparation of endosialin binding molecules of the invention by cell-free translation or synthesized in vitro using techniques that are known to those of skill in the art. For example, the TNT® Quick Coupled Transcription/Translation System (Promega) may be used for cell-free protein expression.

It is likely that antibodies expressed by different cell lines or in animals will have different glycosylation from each other. However, all antibodies encoded by the nucleic acid molecules provided herein, or comprising the amino acid sequences provided herein are part of the instant invention, regardless of the glycosylation of the antibodies.

Anti-endosialin antibodies of the invention can be produced in a non-human mammal or a plant that is transgenic for the nucleic acid(s) (such as those encoding heavy and light chain sequences or antigen-binding portions) of interest. In the case of non-human mammals, endosialin binding molecules of the invention can be produced in and recovered from the blood, milk, urine, saliva, tears, mucus and other bodily fluids of mice, rats, sheep, pigs, goats, cattle, horses or other non-human mammals. See, e.g., U.S. Pat. Nos. 5,827,690, 5,756,687, 5,750,172, and 5,741,957, incorporated herein by reference in their entirety. Methods for making proteins, such as antibodies, in plants are described, e.g., in U.S. Pat. Nos. 6,046,037 and 5,959,177, incorporated herein by reference in their entirety.

In some embodiments, non-human transgenic animals or plants are produced by introducing one or more nucleic acid molecules encoding an anti-endosialin antibody of the invention into the animal or plant by standard transgenic techniques. See Hogan et al., Manipulating the Mouse Embryo: A Laboratory Manual second ed., Cold Spring Harbor Press (1999) and U.S. Pat. No. 6,417,429. The transgenic cells used for making the transgenic animal can be embryonic stem cells or somatic cells or a fertilized egg. The transgenic non-human organisms can be chimeric, nonchimeric heterozygotes, and nonchimeric homozygotes. See, e.g., Hogan et al., Manipulating the Mouse Embryo: A Laboratory Manual second ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999), all incorporated herein by reference. According to the invention, transgenic non-human mammals may comprise and express nucleic acid molecules encoding heavy and light chains or antigen-binding portions that specifically bind to endosialin, and preferably bind to the extracellular domain of endosialin, particularly human endosialin. In some embodiments, the transgenic animals comprise nucleic acid molecules encoding a modified antibody such as a single-chain antibody, a chimeric antibody or a humanized antibody.

In another aspect, the invention provides a method for converting the class or subclass of an anti-endosialin antibody to another class or subclass. In some embodiments, a nucleic acid molecule encoding a V_{L} or V_{H} that does not include sequences encoding C_{L} or C_{H} is isolated using methods well-known in the art. The nucleic acid molecule then is operably linked to a nucleotide sequence encoding a C_{L} or C_{H} from a desired immunoglobulin class or subclass. This can be achieved using a vector or nucleic acid molecule that comprises a C_{L} or C_{H} chain, as described herein. For example, an anti-endosialin antibody that was originally IgM can be class switched to an IgG. Further, the class switching may be used to convert one IgG subclass to another, e.g., from IgG1 or IgG2 to IgG4.

A nucleic acid encoding a heavy chain or a light chain of an anti-endosialin antibody of the invention or an antigen-binding portion of such and antibody may be deimmunized to reduce its immunogenicity using the techniques such as those described in, e.g., International Patent Publication Nos. WO 98/52976 and WO 00/34317, United States Patent Publications 20030153043 and 20080206239 (incorporated herein by reference in their entirety). In some cases, the nucleic acid may be mutated to alter or remove B cell and T cell epitopes to reduce the immunogenicity of the antibody.

In another embodiment, the nucleic acid molecules, vectors and host cells may be used to make mutated anti-endosialin antibodies. The antibodies may be mutated in the variable domains of the heavy and/or light chains, e.g., to alter a binding property of the antibody. For example, a mutation may be made in one or more of the CDR regions or framework (FW) regions to increase or decrease the K_{D} of the antibody for endosialin, to increase or decrease k_{off}, or to alter the binding specificity of the antibody. Techniques in site-directed mutagenesis are well-known in the art. See, e.g., Sambrook et al. and Ausubel et al., supra. The mutations may be made in a CDR region or framework region of a variable domain, or in a constant domain. In some embodiments, one or more mutations are made at an amino acid residue in a CDR region or framework region that is changed compared to the germline sequence of a variable domain of an amino acid sequence selected from SEQ ID NO:14, SEQ ID NO:46, SEQ ID NO:78, SEQ ID NO:110, SEQ ID NO:157, SEQ ID NO: 200, SEQ ID NO: 232, SEQ ID NO:30, SEQ ID NO:62, SEQ ID NO:94, SEQ ID NO:126, SEQ ID NO:175, SEQ ID NO: 216 or SEQ ID NO: 248. In some embodiments, the mutations are made at amino acid residues outside the CDR regions.

One or more framework region residues may be mutated to the residue(s) occurring in the germline sequence. A mutation may be made in a framework region or constant domain to increase the half-life of the anti-endosialin antibody. See, e.g., International Patent Publication No. WO 00/09560, incorporated herein by reference. A mutation in a framework region or constant domain also can be made to alter the immunogenicity of the antibody, to provide a site for covalent or non-covalent binding to another molecule, to add or remove one or more glycosylation sites or to alter such properties as complement fixation, FcR binding and antibody-dependent cell-mediated cytotoxicity (ADCC). According to the invention, a single antibody may have mutations in any one or more of the CDRs or framework regions of the variable domain or in the constant domain.

In some embodiments, there are from 1 to 8, including any number in between, amino acid mutations in either the V_{H} or V_{L} domains of the mutated anti-endosialin antibody compared to the anti-endosialin antibody prior to mutation. In any of the above, the mutations may occur in one or more CDR regions. Further, any of the mutations can be conservative amino acid substitutions. In some embodiments, there are no more than 5, 4, 3, 2, or 1 amino acid changes in the constant domains.

In another aspect, the invention provides a fusion antibody or immunoadhesin may be made that comprises all or a portion of an anti-endosialin antibody of the invention linked to another (non-immunogloblulin) polypeptide. In some embodiments, only the variable domains of the anti-endosialin antibody are linked to the non-immunoglobulin polypeptide. The V_{H} domain of an anti-endosialin antibody may be linked to a first polypeptide, while the V_{L} domain of an anti-endosialin antibody is linked to a second polypeptide that associates with the first polypeptide in a manner such that the V_{H} and V_{L} domains can interact with one another to form an antigen binding site. In still other embodiments, a V_{H} domain is joined to a V_{L} domain by a linker such that the V_{H} and V_{L} domains can interact with one another. The V_{H}-linker-V_{L} antibody may then be linked to a polypeptide of interest. Such fusion antibodies are useful for directing a polypeptide to an endosialin-expressing cell or tissue. The non-immunoglobulin polypeptide may be a therapeutic agent, such as a toxin, chemokine or other regulatory protein, or may be a diagnostic agent, such as an enzyme that may be easily visualized, such as horseradish peroxidase. In addition, fusion antibodies can be created in which two (or more) single-chain antibodies are linked to one another. This is useful if one wants to create a divalent or polyvalent antibody on a single polypeptide chain, or if one wants to create a bispecific antibody.

To create a single chain antibody, (scFv) the V_{H}- and V_{L}-encoding DNA fragments are operably linked to another fragment encoding a flexible linker, e.g., encoding the amino acid sequence (Gly₄-Ser)₃ (SEQ ID NO: 260), such that the V_{H} and V_{L} sequences can be expressed as a contiguous single-chain protein, with the V_{L} and V_{H} domains joined by the flexible linker. See, e.g., Bird et al., Science 242:423-426 (1988); Huston et al., Proc. Natl. Acad. Sci. USA 85:5879-5883 (1988); McCafferty et al., Nature 348:552-554 (1990). The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to endosialin and to another molecule.

In other embodiments, other modified antibodies may be prepared using anti-endosialin antibody-encoding nucleic acid molecules. For instance, “Kappa bodies” (Ill et al., Protein Eng. 10: 949-57 (1997)), “Minibodies” (Martin et al., EMBO J. 13: 5303-9 (1994)), “Diabodies” (Holliger et al., Proc. Natl. Acad. Sci. USA 90: 6444-6448 (1993)), or “Janusins” (Traunecker et al., EMBO J. 10:3655-3659 (1991) and Traunecker et al., Int. J. Cancer (Suppl.) 7:51-52 (1992)) may be prepared using standard molecular biological techniques following the teachings of the specification.

Bispecific antibodies or antigen-binding fragments can be produced by a variety of methods including fusion of hybridomas or linking of Fab′ fragments. See, e.g., Songsivilai & Lachmann, Clin. Exp. Immunol. 79: 315-321 (1990), Kostelny et al., J. Immunol. 148:1547-1553 (1992). In addition, bispecific antibodies may be formed as “diabodies” or “Janusins.” In some embodiments, the bispecific antibody binds to two different epitopes of endosialin. In some embodiments, the bispecific antibody has a first heavy chain and a first light chain from antibodies 1-3-1, 1-25-2, 1-55-2, Hu 1-3-1, Hu 1-25-2 or Hu 1-55-2, and an additional antibody heavy chain and light chain. In some embodiments, the additional light chain and heavy chain also are from one of the above-identified monoclonal antibodies, but are different from the first heavy and light chains.

In some embodiments, the modified antibodies described herein are prepared using one or more of the variable domains or CDR regions from an anti-endosialin antibody provided herein.

According to the invention, an anti-endosialin antibody or antigen-binding portion of the invention can be derivatized or linked to another molecule (e.g., another peptide or protein). In general, the antibodies or portions thereof are derivatized such that the endosialin binding is not affected adversely by the derivatization or labeling. Accordingly, the antibodies and antibody portions of the invention are intended to include both intact and modified forms of the anti-endosialin antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antigen-binding portion of the invention may be derivatized include fluorescent compounds, including fluorescein, fluorescein isothiocyanate, rhodamine, phycoerythrin, 5-dimethylamine-1-napthalenesulfonyl chloride, lanthanide phosphors and the like. An antibody can also be labeled with enzymes that are useful for detection, such as horseradish peroxidase, β-galactosidase, luciferase, alkaline phosphatase, glucose oxidase and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable. An antibody can also be labeled with biotin, and detected through indirect measurement of avidin or streptavidin binding. An antibody can also be labeled with a predetermined polypeptide epitope recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

An anti-endosialin antibody can also be labeled with a radiolabeled amino acid. The radiolabel can be used for both diagnostic and therapeutic purposes. For instance, the radiolabel can be used to detect endosialin-expressing tumors by x-ray or other diagnostic techniques. Further, the radiolabel can be used therapeutically as a toxin for cancerous cells or tumors. Examples of labels for polypeptides include, but are not limited to, the following radioisotopes or radionuclides—³H, ¹⁴C, ¹⁵N, ³⁵S, ⁹⁰Y, ⁹⁹Tc, ¹¹¹In, ¹²⁵I, and ¹³¹I.

An anti-endosialin antibody can also be derivatized with a chemical group such as polyethylene glycol (PEG), a methyl or ethyl group, or a carbohydrate group. These groups are useful to improve the biological characteristics of the antibody, e.g., to increase serum half-life or to increase tissue binding.

In some embodiments, the anti-endosialin antibody can be labeled with a paramagnetic, radioactive or fluorogenic ion that is detectable upon imaging.

In some embodiments, the paramagnetic ion is chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). In other embodiments, the radioactive ion is iodine123, technetium99, indium111, rhenium188, rhenium186, copper67, iodine131, yttrium90, iodine125, astatine211, and gallium67. In other embodiments, the anti-endosialin antibody is labeled with an X-ray imaging agent such as lanthanum (III), gold (III), lead (II) and bismuth (III).

In a further aspect, the invention provides a composition comprising an endosialin binding molecule, such as an antibody of the invention that specifically binds endosialin or an antigen-binding portion of such an antibody. Where the binding molecule is an antagonist, the composition is useful to treat a subject with a condition in which endosialin has a role, including, but not limited to, cancer, tumor growth, conditions involving pathogenic angiogenesis, neoplastic disorders, hyperproliferative disorders, and inflammatory disease. In some embodiments, the composition may be used to treat a subject with colo-rectal cancer, non-small cell lung carcinoma, melanoma, breast cancer, sarcoma, renal cell carcinoma, ovarian cancer or endometrial cancer. In some embodiments, the subject of treatment is a human. In other embodiments, the subject is a veterinary subject.

Treatment with an antagonist endosialin binding molecule of the invention may reduce tumor growth, angiogenesis, and/or inflammation in connective tissue, cartilage, liver, lung, kidney, neural tissue including brain, spinal cord, and peripheral neural tissue, heart, blood vessels, esophagus, stomach, small intestine, large intestine, colon, prostate, pancreas, urinary tract, ovaries, breasts, uterus, testis, penis, bone, muscle, thyroid gland, adrenal gland, pituitary, adipose tissue, bone marrow, blood, thymus, spleen, lymph nodes, skin, eye, ear or nose. The tissues also may be ones having mucosal surfaces.

The composition may comprise a pharmaceutically acceptable carrier or vehicle. A “pharmaceutically acceptable carrier” may be a solvent, dispersion media, coatings, antibacterial and antifungal agents, isotonic and absorption delaying agents, and the like that are physiologically compatible. Some examples of pharmaceutically acceptable carriers merely by way of illustration, are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

A composition of this invention may be in any suitable form for administration to a subject, for example, liquid, semi-solid and solid dosage forms, such as liquid solutions (e.g., injectable and infusible solutions), dispersions or suspensions, aerosols, tablets, pills, powders, liposomes and suppositories. The form depends on the intended mode of administration and therapeutic application. Typical compositions are in the form of injectable or infusible solutions, such as compositions similar to those used for passive immunization of humans. The preferred mode of administration is parenteral (e.g., intravenous, subcutaneous, intraperitoneal, intramuscular) such as by intravenous infusion or injection but administration by intramuscular or subcutaneous injection, oral and nasal routes also is contemplated. Other modes of administration contemplated by the invention in include intrabronchial, transmucosal, intraspinal, intrasynovial, intraaortic, ocular, otic, topical and buccal, and intratumoral.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the anti-endosialin antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the active compound of the antibody compositions may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978).

Additional active compounds also can be incorporated into the compositions. In certain embodiments, an inhibitory anti-endosialin antibody or antigen-binding portion of the invention is co-formulated with and/or co-administered with one or more additional therapeutic, diagnostic, or prophylactic agents. Therapeutic agents include, without limitation, an anti-endosialin antibody with a different fine specificity, antibodies that bind other targets, photosensitizers, nonsteroidal antiinflammatory agents, antihypertensive agents, analgesic agents, antibiotics, anticancer agents, anesthetics, antiemetics, steroids, anti-allergy agents, chemotherapeutic agents, agents for smoking cessation, anti-viral agents, immunosuppresants, antineoplastic agents and cytotoxic agents.

According to the invention, an anti-endosialin antibody of the invention may be co-formulated with an antibody or other agent that is known to inhibit tumor or cancer cell proliferation, e.g., an antibody or agent that inhibits erbB2 receptor, E-selectin, EGF-R, CD20, VEGF (for example, AVASTIN® (bevacizumab), LUCENTIS® (ranibizumab) and MACUGEN® (pegaptanib)), VEGF receptor 1 (VEGFR1), VEGF receptor 2 (VEGFR2) or VEGF receptor 3 (VEGFR3).

Examples of chemotherapeutic agents include, without limitation, GLEEVEC® (imatinib), ERBITUX® (cetuximab), L-asparaginase, IRESSA® (gefitinib), TARCEVA® (erlotinib) and VELCADE® (bortezomib) and the like.

More specifically, the anti-endosialin antibody of the invention may be co-formulated with alkylating agents. Examples of useful alkylating agents include, without limitation, altretamine (hexamethylmelamine), busulfan, carboplatin, carmustine (BCNU), chlorambucil, cisplatin, CYTOXAN® (cyclophosphamide), dacarbazine (DTIC), ifosfamide, lomustine, mechlorethamine (nitrogen mustard), melphalan, oxalaplatin, streptozocin, TEMODAR® (temozolomide) and thiotepa and the like.

The anti-endosialin antibody of the invention may be co-formulated with antimetabolites. Examples of useful antimetabolites include, without limitation, 5-fluorouracil (5-FU), 6-mercaptopurine (6-MP), XELODA® (capecitabine), ARA-C® (cytarabine), fludarabine, GEMZAR® (gemcitabine), methotrexate and ALIMTA® (pemetrexed) and the like.

The anti-endosialin antibody of the invention may be co-formulated with topoisomerase I and II inhibitors, including, without limitation, CAMPTOSAR® (irinotecan HCl), SN-38, camptothecin, HYCAMTIN® (topotecan), etoposide, teniposide, ELLENCE® (epirubicin), ADRIAMYCIN® (doxorubicin), idarubicin, mitoxantrone, lamellarin D and HU-331 (Kogan et al. (2007) Molecular Cancer Therapeutics 6: 173-183, incorporated herein by reference) and the like.

In some embodiments, the anti-endosialin antibody of the invention may be co-formulated with anti-tumor antibiotics, such as actinomycin-D, bleomycin, and mitomycin-C and the like.

In some embodiments, the anti-endosialin antibody of the invention may be co-formulated with mitotic inhibitors. Non-limiting examples of useful mitotic inhibitors include EMCYT® (estramustine), IXEMPRA® (ixabepilone), TAXOTERE® (docetaxel), TAXOL® (paclitaxel), VELBAN® (vinblastine), ONCOVIN® (vincristine), and NAVELBINE® (vinorelbine) and the like.

In some embodiments, the anti-endosialin antibody of the invention may be co-formulated with differentiating agents. Non-limiting examples of useful differentiating agents include arsenic trioxide, retinoids, tretinoin and TARGRETIN® (bexarotene) and the like.

In some embodiments, the anti-endosialin antibody of the invention may be co-formulated with steroid compounds, such as, for example, prednisone, methylprednisolone and dexamethasone and the like.

In some embodiments, the anti-endosialin antibody of the invention may be co-formulated with hormone-related compounds. Non-limiting examples of useful hormone-related compounds include estrogens, progestins (such as MEGACE® (megestrol acetate)), FASLODEX® (fulvestrant), tamoxifen, toremifene, LUPRON® (leuprolide), ZOLADEX® (goserelin), ARIMIDEX® (anastrozole), FEMARA® (letrozole), AROMASIN® (exemestane), CASODEX® (bicalutamide), EULEXIN® (flutamide) and NILANDRON® (nilutamide).

In some embodiments, the anti-endosialin antibody of the invention may be co-formulated with COX-II (cyclooxygenase II) inhibitors. Non-limiting examples of useful COX-II inhibitors include CELEBREX® (celecoxib), valdecoxib, and rofecoxib and the like.

In some embodiments, the anti-endosialin antibody of the invention may be co-formulated with immunotherapeutic agents. Non-limiting examples of useful immunotherapeutic agents include the interferons (such as interferon-alpha), BCG, interleukin-2 (IL-2), thalidomide, lenalidomide, CAMPATH® (alemtuzumab) and RITUXAN® (rituximab) and the like.

In some embodiments, the anti-endosialin antibody of the invention may be co-formulated with an MMP inhibitor. For example, the anti-endosialin antibody may be co-formulated with anti-angiogenic agents, such as MMP-2 (matrix-metalloproteinase 2) inhibitors or MMP-9 (matrix-metalloproteinase 9) inhibitors. Preferred MMP inhibitors are those that do not demonstrate arthralgia. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, RS 13-0830, and the compounds recited in the following list: 3-[[4-(4-fluoro-phenoxy)-benzenesulfonyl]-(1-hydro-xycarbamoyl-cyclopentyl)-amino]-propionic acid; 3-exo-3-[4-(4-fluoro-pheno-xy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl-]-3-hydroxy-3-methyl-piperidine-2-carboxylic acid hydroxyamide; 4-[4-(4-fluoro-phenoxy)-benzenesulfonylamino]-tetrahydro-pyran-4-carboxyl-ic acid hydroxyamide; 3-[[4-(4-fluoro-phenoxy)-benzenesulfonyl]-(1-hydroxy-carbamoyl-cyclobutyl)-amino]-propionic acid; 4-[4-(4-chloro-phenoxy)-benze-nesulfonylamino]-tetrahydro-pyran-4-carboxylic acid hydroxyamide; (R) 3-[4-(4-chloro-phenoxy)-benzenesulfonylamino]-tetrahydro-pyran-3-carboxyl-ic acid hydroxyamide; (2R,3R) 1-[4-(4-fluoro-2-methyl-benzyloxy)-benzenes-ulfonyl]-3-hydroxy-3-methyl-piperidine-2-carboxylic acid hydroxyamide; 3-[[4-(4-fluoro-phenoxy)-benzenesulfonyl]-(1-hydroxycarbamoyl-1-methyl-et-hyl)-amino]-propionic acid; 3-[[4-(4-fluoro-phenoxy)-benzenesulfonyl]-(4-h-ydroxycarbamoyl-tetrahydro-pyran-4-yl)-amino]-propionic acid; 3-exo-3-[4-(4-chloro-phenoxy)-benzenesulfonylamino]-8-oxa-icyclo[3.2.1]oc-tane-3-carboxylic acid hydroxyamide; 3-endo-3-[4-(4-fluoro-phenoxy)-benze-nesulfonylamino]-8-oxa-icyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide and the like; and pharmaceutically acceptable salts and solvates of said compounds.

In some embodiments, the anti-endosialin antibody may be co-formulated with an integrin inhibitor. Integrin inhibitors, include, without limitation, obtustatin, rhodocetin, Vitaxin (Medlmmune), cilengitide (EMD 121974; Merck), S137 (Pfizer), S247 (Pfizer) and JSM6427 (Jerini) (see, e.g., Brown et al. (2008) International Journal of Cancer 123: 2195-2203; Stupp et al. (2007) Journal of Clinical Oncology 25: 1637-1638; Eble et al. (2003) Biochem J. 376: 77-85, all incorporated herein by reference).

The compositions of the invention may include a “therapeutically effective amount” or a “prophylactically effective amount” of an antibody or antigen-binding portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the anti-endosialin antibody or portion thereof and the particular therapeutic or prophylactic effect to be achieved, and (b) the limitations inherent in the art of compounding such an antibody for the treatment of sensitivity in individuals.

An exemplary, non-limiting range for a therapeutically or prophylactically-effective amount of an antibody or antibody portion of the invention is 0.025 to 50 mg/kg, 0.1 to 50 mg/kg, 0.1-25 mg/kg, 0.1 to 10 mg/kg or 0.1 to 3 mg/kg. In one embodiment, the antibody is administered in a formulation as a sterile aqueous solution having a pH that ranges from about 5.0 to about 6.5 and comprising from about 1 mg/ml to about 200 mg/ml of antibody, from about 1 millimolar to about 100 millimolar of Tween, from about 0.01 mg/ml to about 10 mg/ml of polysorbate 80 or polysorbate 20, from about 100 millimolar to about 400 millimolar of a non-reducing sugar selected from but not limited to trehalose or sucrose, from about 0.01 millimolar to about 1.0 millimolar of disodium EDTA dihydrate and optionally comprise a pharmaceutically acceptable antioxidant in addition to a chelating agent. Suitable antioxidants include, but are not limited to, methionine, sodium thiosulfate, catalase, and platinum. For example, the composition may contain methionine in a concentration that ranges from 1 mM to about 100 mM, and in particular, is about 27 mM. In some embodiments, a formulation contains 5 mg/ml of antibody in a buffer of 20 mM sodium citrate, pH 5.5, 140 mM NaCl, and 0.2 mg/ml polysorbate 80. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Another aspect of the present invention provides kits comprising an anti-endosialin antibody, or antigen-binding portion, of the invention or a composition comprising such an antibody or antigen-binding portion. A kit may include, in addition to the antibody or composition, diagnostic or therapeutic agents. A kit can also include instructions for use in a diagnostic or therapeutic method, as well as packaging material such as, but not limited to, ice, dry ice, styrofoam, foam, plastic, cellophane, shrink wrap, bubble wrap, cardboard and starch peanuts. In one embodiment, the kit includes the antibody or a composition comprising it and a diagnostic agent that can be used in a method described herein. In still another embodiment, the kit includes the antibody or a composition comprising it and one or more therapeutic agents that can be used in a method described herein.

The invention also relates to compositions and kits for inhibiting cancer in a mammal comprising an amount of an antibody of the invention in combination with an amount of a chemotherapeutic agent, wherein the amounts of the compound, salt, solvate, or prodrug, and of the chemotherapeutic agent are together effective in inhibiting abnormal cell growth. Many chemotherapeutic agents are presently known in the art. In some embodiments, the chemotherapeutic agent is selected from the group consisting of mitotic inhibitors, alkylating agents, anti-metabolites, intercalating antibiotics, chemokine inhibitors, cell cycle inhibitors, enzymes, topoisomerase inhibitors, biological response modifiers, anti-hormones, e.g., anti-androgens, and anti-angiogenesis agents.

The anti-endosialin antibodies may be used for in vitro or in vivo detection of endosialin in a biological sample. The anti-endosialin antibodies may be used in a conventional immunoassay, including, without limitation, an ELISA, an RIA, flow cytometry, immunocytochemistry, tissue immunohistochemistry, Western blot or immunoprecipitation. The anti-endosialin antibodies of the invention may be used to detect endosialin from humans.

In another aspect, the invention provides a method for detecting endosialin in a biological sample. The method comprises contacting a biological sample with an anti-endosialin antibody of the invention and detecting the bound antibody. The anti-endosialin antibody may be directly labeled with a detectable label or may be unlabeled. If an unlabeled antibody is used, a second antibody or other molecule that can bind the anti-endosialin antibody that is labeled is used to detect antibody bound to endosialin. As is well known to one of skill in the art, a second antibody is chosen that is able to specifically bind the specific species and class of the first antibody. For example, if the anti-endosialin antibody comprises a human IgG, then the secondary antibody may be a labeled anti-human-IgG antibody. Other molecules that can bind to antibodies include, without limitation, Protein A and Protein G, both of which are available commercially, e.g., from Pierce Chemical Co.

Suitable labels for the antibody or secondary molecule have been disclosed supra, and include various enzymes, prosthetic groups, fluorescent materials, luminescent materials, magnetic agents and radioactive materials. Examples of suitable enzymes include horseradish peroxidase, alkaline phosphatase, O-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; an example of a magnetic agent includes gadolinium; and examples of suitable radioactive material include ¹²⁵I, ¹³¹I, ³⁵S or ³H.

The anti-endosialin antibodies of the invention may be used to determine the level of endosialin in a tissue or in cells derived from the tissue. The tissue may be a diseased tissue such as a tumor or a biopsy thereof. The detection may be in a tissue sample or in vivo. An anti-endosialin antibody of the invention or an antigen-binding portion of such an antibody may be used according to the invention to detec and/or quantify endosialin in a tissue, cell surface levels of endosialin or localization of endosialin by the methods discussed above.

A preferred immunoassay for determining the localization of endosialin, e.g., cell surface levels, is an immunohistochemistry (IHC)/immunocytochemistry (ICC) assay. Immunohistochemistry and immunocytochemistry procedures are well known in the art. See, e.g., Harlow and Lane, supra. The tissue or cells to be tested may be fixed utilizing any of a variety of fixation conditions which include, without limitation, paraformaldehyde in phosphate buffer, paraformaldehyde in periodate/lysine/phosphate buffer, paraformaldehyde with glutaraldehyde (for Transmission Electron Microscopy), or cold acetone or alcohol (for frozen samples). The tissue also may be embedded in an embedding medium for IHC. Embedding media for immunohistochemistry experiments may include, but are not limited to, paraffin wax or any form of cryomatrix (for frozen samples). To obtain three-dimensional images, it may also be possible to perform immunohistochemistry experiments using a whole-mount preparation. Antigen retrieval, when necessary, may be accomplished by performing Heat Induced Epitope Retrieval (HIER) or Proteolytic Induced Epitope Retrieval (PIER) or a combination thereof. Antibody penetration of cells or tissues for IHC/ICC, when necessary, may be accomplished using a variety of reagents including, without limitation, Triton X-100, saponin or sodium borohydride. Blocking treatment, if necessary, may be performed by treating cells or tissue with a variety of blocking reagents including, but not limited to, serum albumin. The methods of detection of endosialin in a tissue or cell sample are numerous, and may include, without limitation, direct antibody detection, indirect antibody detection, peroxidase anti-peroxidase method, avidin-biotin complex method, labeled streptavidin biotin method or any one of a variety of polymeric signal amplification methods.

An anti-endosialin antibody or antigen-binding portion of the invention may be used for double staining of cells or tissues in combination with a second antibody that recognizes an antigen other than endosialin. For IHC of ICC, an anti-endosialin antibody or antigen-binding portion may be used at a concentration of up to 0.001 μg/ml, 0.005 μg/ml, 0.01 μg/ml, 0.05 μg/ml, 0.1 μg/ml, 0.5 μg/ml, 1 μg/ml, 2.5 μg/ml, 5 μg/ml, 10 μg/ml, 15 μg/ml, 20 μg/ml, or 25 μg/ml, or at a dilution of 1:10,000, 1:1000, 1:750, 1:500, 1:250, 1:200, 1:100, 1:75, 1:50, 1:10, or 1:5. Antibody staining of cells or tissues for IHC or ICC may be for 0.5 minute, 1 minute, 2 minutes, 5 minutes, 10 minutes, 20 minutes, 30 minutes, 45 minutes, 1 hour, 1.5 hours, 2 hours, or 5 hours before being washed off. Because of the surprising potency of the rabbit and humanized anti-endosialin antibodies of the invention in IHC with FFPE tissue samples, antibody concentrations and staining time are reduced.

Endosialin in cells and tissues also may be measured with ELISA or Western blot assays utilizing an anti-endosialin antibody of the invention or an antigen-binding portion thereof. A preferred immunoassay for measuring cell surface endosialin includes the steps of labeling the cell surface proteins with a detectable label, such as biotin or ¹²⁵I, immunoprecipitating the endosialin with an anti-endosialin antibody and then detecting the labeled endosialin. Methods such as ELISA, RIA, Western blot, cell surface labeling of integral membrane proteins and immunoprecipitation are well known in the art. See, e.g., Harlow and Lane, supra. In addition, high throughput screening may be performed by scaling up any one of the above immunoassays in order to test a large number of compounds for either activation or inhibition of endosialin.

The invention also contemplates methods for measuring endosialin in cells or tissues using an anti-endosialin antibody of the invention in a competition immunoassay. A competition immunoassay utilizes endosialin standards labeled with a detectable substance and an unlabeled anti-endosialin antibody to assay endosialin in a biological sample. In this assay, the biological sample, the labeled endosialin standards and the anti-endosialin antibody are combined and the amount of labeled endosialin standard bound to the unlabeled antibody is determined. The amount of endosialin in the biological sample is inversely proportional to the amount of labeled endosialin standard bound to the anti-endosialin antibody.

Any of the above-mentioned immunoassays may be used to detect or measure endosialin in cells in cell culture, for example to identify compounds that activate or inhibit endosialin. Cell surface endosialin or total endosialin may be measured. To measure total endosialin, cells are lysed and the total endosialin level is measured using one of the immunoassays described above.

The antibodies of the present invention, especially humanized antibodies also may be used in vivo to detect endosialin in tissues and organs, for example in endosialin-expressing tumors. For in vivo detection of endosialin, a labeled endosialin binding molecule, such as an antibody or an antigen-binding portion of an antibody is administered to a patient in need of such a diagnostic test and subjecting the patient to imaging analysis in order to determine the location of the endosialin-expressing tissues. Imaging analysis is well known in the medical art, and includes, without limitation, x-ray analysis, magnetic resonance imaging (MRI) or computed tomography (CE). In another embodiment of the method, a tumor or tissue biopsy is obtained from the patient to determine whether it expresses endosialin. For imaging, the anti-endosialin antibody may be labeled with a detectable agent that can be imaged in a patient. For example, the antibody may be labeled with a contrast agent, such as barium, which can be used for x-ray analysis, or a magnetic contrast agent, such as a gadolinium chelate, which can be used for MRI or CE. Other labeling agents include, without limitation, radioisotopes, such as ⁹⁹Tc. According to the invention, the anti-endosialin antibody could also be unlabeled and imaging is by administering a second antibody or other molecule that is detectable and that can bind the anti-endosialin antibody.

Using any of the above-described diagnostic methods, one can determine whether a tissue such as a tumor expresses endosialin such that the patient could be a candidate for treatment with an antagonist endosialin binding molecule of the invention, including an antagonist rabbit anti-endosialin monoclonal antibody, a humanized rabbit anti-endosialin antibody or an antigen-binding portion thereof. Accordingly, the invention encompasses a method for identifying a subject who is a candidate for treatment with an antagonist endosialin binding molecule of the invention, such as an antagonist anti-endosialin antibody or an antigen-binding portion of such an antibody, comprising the step of detecting the presence or absence of endosialin in a tissue of the subject or in a biological sample and identifying the subject as a candidate for treatment with an antagonist endosialin binding molecule of the invention if endosialin is present in the tissue or sample.

The invention further contemplates a method for informing a treatment decision for a subject comprising obtaining information about whether the subject is suffering from a condition that is mediated in whole or in part by endosialin by detecting the presence or absence of endosialin in an appropriate tissue in the subject or in an appropriate biological sample, such as a tissue sample from the subject and electing therapy with an antagonist endosialin binding molecule of the invention if endosialin is present in the tissue or sample. The invention also contemplates a method for determining the amount of an antagonist anti-endosialin antibody of the invention by measuring the level of endosialin expression and/or activity. The invention further contemplates a method for monitoring treatment of a subject with an antagonist endosialin binding molecule of the invention comprising detecting or measuring endosialin in a tissue in the subject or in a tissue sample and comparing the amount of endosialin to the amount in a pretreatment or an earlier post-treatment sample from the subject wherein reduced endosialin compared to the earlier sample indicates that the antagonist endosialin binding molecule is effective to reduce endosialin in the subject.

Still further, according to the invention, any of the above-mentioned methods for detecting endosialin may be used as an indicator of tumor angiogenesis, tumor migration and/or tumor invasion.

In another embodiment, the invention provides methods for inhibiting endosialin activity comprising contacting or exposing a cell expressing endosialin with or to an antagonist endosialin binding molecule, including an anti-endosialin antibody or an antigen binding portion of such an antibody. In some methods, the antagonist endosialin binding molecule is administered to a subject in need thereof. The subject may be suffering from a disease or condition characterized by pathogenic angiogenesis or endosialin-mediated abnormal cellular growth. Non-limiting examples include cancer, tumor growth, angiogenic conditions, neoplastic disorders, and hyperproliferative disorders. The subject may be a human subject or a veterinary subject, including a non-human animal model of a human disease.

According to the methods of the invention, an antagonist endosialin binding molecule of the invention can be administered neat or may be incorporated into a pharmaceutical composition suitable for administration to a subject. The pharmaceutical composition may comprise a pharmaceutically acceptable carrier such as a solvent, dispersion media, coatings, antibacterial and antifungal agents, isotonic and absorption delaying agents, and the like that are physiologically compatible. Examples of pharmaceutically acceptable carriers include but are not limited to one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The antagonist may be administered once or multiple times. Where multiple administrations are used, they may be daily, weekly, monthly or if any appropriate periodically including multiple daily doses. The administering may be on a schedule such as three times daily, twice daily, once daily, once every two days, once every three days, once weekly, once every two weeks, once every month, once every two months, once every three months and once every six months. The antibody may also be administered continuously, e.g. via a minipump. The antibody may be administered, for example, via a mucosal, buccal, intranasal, inhalable, intravenous, subcutaneous, intramuscular, parenteral, or intratumor route. The antibody may be administered once, at least twice or for at least the period of time until the condition is treated, palliated or cured. The antibody generally will be administered for as long as the condition is present or longer to prevent recurrence of the condition. The antibody will generally be administered as part of a pharmaceutical composition as described supra. The dosage of antibody will generally be in the range of 0.1 to 100 mg/kg, more preferably 0.5 to 50 mg/kg, more preferably 1 to 20 mg/kg, and even more preferably 1 to 10 mg/kg. The serum concentration of the antibody may be measured by any method known in the art.

In another embodiment, the anti-endosialin antibody may be co-administered with another therapeutic agent including another antagonist endosialin binding molecule. The additional therapeutic agent also may be an oligonucleotide that reduces expression of endosialin or of a nucleic acid that encodes a product that promotes angiogenesis by RNA interference, including single stranded or double stranded nucleic acid molecules. In the case of a subject suffering from hyperproliferative disorder, such as cancer or a tumor, the additional therapeutic agent may be an antineoplastic agent. In one aspect, the invention relates to a method for the treatment of a hyperproliferative disorder in a subject in need thereof comprising administering to said subject a therapeutically effective amount of an antagonist endosialin binding molecule of the invention in combination with an anti-tumor agent selected from the group consisting of, but not limited to, mitotic inhibitors, alkylating agents, anti-metabolites, intercalating agents, growth factor inhibitors, cell cycle inhibitors, enzymes, topoisomerase inhibitors, biological response modifiers, anti-hormones, kinase inhibitors, matrix metalloprotease inhibitors, genetic therapeutics, anti-androgens, antineoplastic agents and cytotoxic agents. In another preferred embodiment, the antibody or combination therapy is administered along with radiotherapy, chemotherapy, photodynamic therapy, surgery or other immunotherapy.

According to the invention, an anti-endosialin antibody of the invention may be administered with an antibody or other agent that is known to inhibit tumor or cancer cell proliferation, e.g., an antibody or agent that inhibits erbB2 receptor, E-selectin, EGF-R, CD20, VEGF (for example, AVASTIN® (bevacizumab), LUCENTIS® (ranibizumab) and MACUGEN® (pegaptanib)), VEGF receptor 1 (VEGFR1), VEGF receptor 2 (VEGFR2) or VEGF receptor 3 (VEGFR3) and the like.

The anti-endosialin antibody or antigen-binding portion of the invention may be co-administered with chemotherapeutic agents including, without limitation, GLEEVEC® (imatinib), ERBITUX® (cetuximab), L-asparaginase, IRESSA® (gefitinib), TARCEVA® (erlotinib) and VELCADE® (bortezomib) and the like.

More specifically, the anti-endosialin antibody of the invention may be co-administered with alkylating agents. Examples of useful alkylating agents include, without limitation, altretamine (hexamethylmelamine), busulfan, carboplatin, carmustine (BCNU), chlorambucil, cisplatin, CYTOXAN® (cyclophosphamide), dacarbazine (DTIC), ifosfamide, lomustine, mechlorethamine (nitrogen mustard), melphalan, oxalaplatin, streptozocin, TEMODAR® (temozolomide), thiotepa and the like.

The anti-endosialin antibody of the invention may be co-administered with antimetabolites. Examples of useful antimetabolites include, without limitation, 5-fluorouracil (5-FU), 6-mercaptopurine (6-MP), XELODA® (capecitabine), ARA-C® (cytarabine), fludarabine, GEMZAR® (gemcitabine), methotrexate, ALIMTA® (pemetrexed) and the like.

The anti-endosialin antibody of the invention may be co-administered with topoisomerase I and II inhibitors, including, without limitation, CAMPTOSAR® (irinotecan HCl), SN-38, camptothecin, HYCAMTIN® (topotecan), etoposide, teniposide, ELLENCE® (epirubicin), ADRIAMYCIN® (doxorubicin), idarubicin, mitoxantrone, lamellarin D, HU-331 (Kogan et al. (2007) Molecular Cancer Therapeutics 6: 173-183) and the like.

In some embodiments, the anti-endosialin antibody of the invention may be co-administered with anti-tumor antibiotics, such as actinomycin-D, bleomycin, mitomycin-C and the like.

In some embodiments, the anti-endosialin antibody of the invention may be co-administered with mitotic inhibitors. Non-limiting examples of useful mitotic inhibitors include EMCYT® (estramustine), IXEMPRA® (ixabepilone), TAXOTERE® (docetaxel), TAXOL® (paclitaxel), VELBAN® (vinblastine), ONCOVIN® (vincristine), NAVELBINE® (vinorelbine) and the like.

In some embodiments, the anti-endosialin antibody of the invention may be co-administered with differentiating agents. Non-limiting examples of useful differentiating agents include arsenic trioxide, retinoids, tretinoin TARGRETIN® (bexarotene) and the like.

In some embodiments, the anti-endosialin antibody of the invention may be co-administered with steroid compounds, such as, for example, prednisone, methylprednisolone, dexamethasone and the like.

In some embodiments, the anti-endosialin antibody of the invention may be co-administered with hormone-related compounds. Non-limiting examples of useful hormone-related compounds include estrogens, progestins (such as MEGACE® (megestrol acetate)), FASLODEX® (fulvestrant), tamoxifen, toremifene, LUPRON® (leuprolide), ZOLADEX® (goserelin), ARIMIDEX® (anastrozole), FEMARA® (letrozole), AROMASIN® (exemestane), CASODEX® (bicalutamide), EULEXIN® (flutamide), NILANDRON® (nilutamide) and the like.

In some embodiments, the anti-endosialin antibody of the invention may be co-administered with a COX-II (cyclooxygenase II) inhibitor. Non-limiting examples of useful COX-II inhibitors include CELEBREX® (celecoxib), valdecoxib, rofecoxib and the like.

In some embodiments, the anti-endosialin antibody of the invention may be co-administered with immunotherapeutic agents. Non-limiting examples of useful immunotherapeutic agents include the interferons (such as interferon-alpha), BCG, interleukin-2 (IL-2), thalidomide, lenalidomide, CAMPATH® (alemtuzumab), RITUXAN® (rituximab).

In some embodiments, the anti-endosialin antibody of the invention may be co-administered with an MMP inhibitor. For example, the anti-endosialin antibody may be co-administered with anti-angiogenic agents, such as MMP-2 (matrix-metalloproteinase 2) inhibitors or MMP-9 (matrix-metalloproteinase 9) inhibitors. Preferred MMP inhibitors are those that do not demonstrate arthralgia. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, RS 13-0830, and the compounds recited in the following list: 3-[[4-(4-fluoro-phenoxy)-benzenesulfonyl]-(1-hydro-xycarbamoyl-cyclopentyl)-amino]-propionic acid; 3-exo-3-[4-(4-fluoro-pheno-xy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl-]-3-hydroxy-3-methyl-piperidine-2-carboxylic acid hydroxyamide; 4-[4-(4-fluoro-phenoxy)-benzenesulfonylamino]-tetrahydro-pyran-4-carboxyl-ic acid hydroxyamide; 3-[[4-(4-fluoro-phenoxy)-benzenesulfonyl]-(1-hydroxy-carbamoyl-cyclobutyl)-amino]-propionic acid; 4-[4-(4-chloro-phenoxy)-benze-nesulfonylamino]-tetrahydro-pyran-4-carboxylic acid hydroxyamide; (R) 3-[4-(4-chloro-phenoxy)-benzenesulfonylamino]-tetrahydro-pyran-3-carboxyl-ic acid hydroxyamide; (2R,3R) 1-[4-(4-fluoro-2-methyl-benzyloxy)-benzenes-ulfonyl]-3-hydroxy-3-methyl-piperidine-2-carboxylic acid hydroxyamide; 3-[[4-(4-fluoro-phenoxy)-benzenesulfonyl]-(1-hydroxycarbamoyl-1-methyl-et-hyl)-amino]-propionic acid; 3-[[4-(4-fluoro-phenoxy)-benzenesulfonyl]-(4-h-ydroxycarbamoyl-tetrahydro-pyran-4-yl)-amino]-propionic acid; 3-exo-3-[4-(4-chloro-phenoxy)-benzenesulfonylamino]-8-oxa-icyclo[3.2.1]oc-tane-3-carboxylic acid hydroxyamide; 3-endo-3-[4-(4-fluoro-phenoxy)-benze-nesulfonylamino]-8-oxa-icyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of said compounds.

In some embodiments, the anti-endosialin antibody may be co-administered with an integrin inhibitor. Integrin inhibitors, include, without limitation, obtustatin, rhodocetin, Vitaxin (Medlmmune), cilengitide (EMD 121974; Merck), S137 (Pfizer), S247 (Pfizer) and JSM6427 (Jerini) (see, e.g., Brown et al. (2008) International Journal of Cancer 123: 2195-2203; Stupp et al. (2007) Journal of Clinical Oncology 25: 1637-1638; Eble et al. (2003) Biochem J. 376: 77-85, all incorporated herein by reference).

Co-administration of an antibody of the invention or an antigen-binding portion of such antibody with an additional therapeutic agent (combination therapy) encompasses administering a pharmaceutical composition comprising the anti-endosialin antibody and the additional therapeutic agent as well as administering two or more separate pharmaceutical compositions: one comprising the anti-endosialin antibody and the other(s) comprising the additional therapeutic agent(s). Further, co-administration or combination therapy includes the antibody and additional therapeutic agents are administered simultaneously or sequentially, or both. For instance, the antibody may be administered once every three days, while the additional therapeutic agent is administered once daily at the same as the antibody or at a different time. An antagonist antibody may be administered prior to or subsequent to treatment with the additional therapeutic agent, for example, an antagonist antibody of the invention may be administered therapy with the additional agent. Similarly, administration of an antagonist anti-endosialin antibody of the invention may be part of a treatment regimen that includes other treatment modalities including radiation, surgery, exercise, phototherapy, including laser therapy, and dietary supplements. The combination therapy may be administered to prevent recurrence of the condition. Preferably, the combination therapy is administered multiple times. The combination therapy may be administered from three times daily to once every six months. The administering may be on a schedule such as three times daily, twice daily, once daily, once every two days, once every three days, once weekly, once every two weeks, once every month, once every two months, once every three months and once every six months, or may be administered continuously, e.g. via a minipump. The combination therapy may be administered, for example, via an oral, mucosal, buccal, intranasal, inhalable, intravenous, subcutaneous, intramuscular or parenteral route.

In one embodiment, the antibody is administered in a formulation as a sterile aqueous solution having a pH that ranges from about 5.0 to about 8.0, preferably from about 6.5 to about 7.5, and more preferably from about 7.0 to about 7.2. The formulation may comprise from about 1 mg/ml to about 200 mg/ml, from about 5 mg/ml to about 50 mg/ml, or from about 10 mg/ml to about 25 mg/ml, of antibody. The formulation may comprise from about 1 millimolar to about 100 millimolar of Tween, from about 0.01 mg/ml to about 10 mg/ml of polysorbate 80, from about 100 millimolar to about 400 millimolar of trehalose, and from about 0.01 millimolar to about 1.0 millimolar of disodium EDTA dihydrate. In a preferred embodiment, the antibody is administered in a formulation of 5.0±0.5 mg/mL of antibody in 10 mM sodium phosphate, 150 mM sodium chloride, pH 7.2, 0.01% USP Tween 80.

In a still further embodiment, the anti-endosialin antibody is labeled with a radiolabel, an immunotoxin or a toxin, or is a fusion protein comprising a cytotoxic peptide. The anti-endosialin antibody or anti-endosialin antibody fusion protein directs the radiolabel, immunotoxin, toxin or toxic peptide to the endosialin-expressing tumor or cancer cell. In a preferred embodiment, the radiolabel, immunotoxin, toxin or toxic peptide is internalized after the anti-endosialin antibody binds to the endosialin on the surface of the tumor or cancer cell.

It is further contemplated by the present invention that any of the compositions herein may be administered to a subject susceptible to or suffering from a condition associated with pathogenic angiogenesis (“an angiogenic condition”).

Examples of angiogenic conditions that may be treated/prevented by the compositions/methods of the present invention include, but are not limited to, cancer (both solid and hematologic), age-related macular degeneration (AMD), developmental abnormalities (organogenesis), diabetic blindness, endometriosis, ocular neovascularization, psoriasis, rheumatoid arthritis (RA), and skin discolorations (e.g., hemangioma, nevus flammeus, or nevus simplex).

For example, the present invention relates to methods for treating or preventing conditions associated with ocular neovascularization using any of the compositions/methods herein. Conditions associated with ocular neovascularization include, but are not limited to, diabetic retinopathy, age related macular degeneration (“ARMD”), rubeotic glaucoma, interstitial keratitis, retinopathy of prematurity, ischemic retinopathy (e.g., sickle cell), pathological myopic, ocular histoplasmosis, pterygia, punitiate inner choroidopathy, and the like.

The invention further provides a method of reducing endosialin binding to a ligand selected from fibronectin, collagen I and collagen IV, comprising the step of exposing a cell expressing endosialin to an antagonist endosialin binding molecule of the invention under conditions that permit binding of the molecule to endosialin. The endosialin may be human, non-human, primate or mouse endosialin. The binding molecule can be an antagonist anti-endosialin antibody or an antigen-binding portion thereof.

Also encompassed by the invention is a method for reducing endosialin mediated angiogenesis, comprising the step of exposing a cell expressing endosialin to an antagonist endosialin binding molecule of the invention under conditions that permit binding of the molecule to endosialin. The endosialin may be human, non-human, primate or mouse endosialin. The binding molecule can be an antagonist anti-endosialin antibody or an antigen-binding portion thereof.

Further, the invention encompasses a method for reducing growth of an endosialin-expressing tumor, comprising the step of exposing a cell expressing endosialin to an antagonist endosialin binding molecule of the invention under conditions that permit binding of the molecule to endosialin. The endosialin may be human, non-human, primate or mouse endosialin. The binding molecule can be an antagonist anti-endosialin antibody or an antigen-binding portion thereof.

TABLE 1
TABLE OF SEQUENCE IDENTIFIERS (SEQ ID NOs)

| | | 1-3-1 | 1-25-2 | 1-55-2 | Clone 8 | Hu 1-3-1 | Hu 1-25-2 | Hu 1-55-2 |
|---|---|---|---|---|---|---|---|---|
| V_{H} | DNA | 6 | 38 | 70 | 102 | 148 | 192 | 224 |
| | PROT | 14 | 46 | 78 | 110 | 157 | 200 | 232 |
| HFW1 | DNA | 7 | 39 | 71 | 103 | 149 | 193 | 225 |
| | PROT | 15 | 47 | 79 | 111 | 158 | 201 | 233 |
| HCDR1 | DNA | 8 | 40 | 72 | 104 | 150 | 194 | 226 |
| | PROT | 16 | 48 | 80 | 112 | 159 | 202 | 234 |
| HFW2 | DNA | 9 | 41 | 73 | 105 | 151 | 195 | 227 |
| | PROT | 17 | 49 | 81 | 113 | 160 | 203 | 235 |
| HCDR2 | DNA | 10 | 42 | 74 | 106 | 152 | 196 | 228 |
| | PROT | 18 | 50 | 82 | 114 | 161 | 204 | 236 |
| HFW3 | DNA | 11 | 43 | 75 | 107 | 153 | 197 | 229 |
| | PROT | 19 | 51 | 83 | 115 | 162 | 205 | 237 |
| HCDR3 | DNA | 12 | 44 | 76 | 108 | 154 | 198 | 230 |
| | PROT | 20 | 52 | 84 | 116 | 163 | 206 | 238 |
| HFW4 | DNA | 13 | 45 | 77 | 109 | 155 | 199 | 231 |
| | PROT | 21 | 53 | 85 | 117 | 164 | 207 | 239 |
| V_{L} | DNA | 22 | 54 | 86 | 118 | 166 | 208 | 240 |
| | PROT | 30 | 62 | 94 | 126 | 175 | 216 | 248 |
| LFW1 | DNA | 23 | 55 | 87 | 119 | 167 | 209 | 241 |
| | PROT | 31 | 63 | 95 | 127 | 176 | 217 | 249 |
| LCDR1 | DNA | 24 | 56 | 88 | 120 | 168 | 210 | 242 |
| | PROT | 32 | 64 | 96 | 128 | 177 | 218 | 250 |
| LFW2 | DNA | 25 | 57 | 89 | 121 | 169 | 211 | 243 |
| | PROT | 33 | 65 | 97 | 129 | 178 | 219 | 251 |
| LCDR2 | DNA | 26 | 58 | 90 | 122 | 170 | 212 | 244 |
| | PROT | 34 | 66 | 98 | 130 | 179 | 220 | 252 |
| LFW3 | DNA | 27 | 59 | 91 | 123 | 171 | 213 | 245 |
| | PROT | 35 | 67 | 99 | 131 | 180 | 221 | 253 |
| LCDR3 | DNA | 28 | 60 | 92 | 124 | 172 | 214 | 246 |
| | PROT | 36 | 68 | 100 | 132 | 181 | 222 | 254 |
| LFW4 | DNA | 29 | 61 | 93 | 125 | 173 | 215 | 247 |
| | PROT | 37 | 69 | 101 | 133 | 182 | 223 | 255 |

It is understood that the examples and embodiments described herein are for illustrative purposes only and that various modifications or changes in light thereof will be apparent to persons skilled in the art and are to be included within the and can be made without departing from the true scope of the invention.

### EXAMPLES

### Example 1

### Generation of Rabbit Anti-hEndosialin MAbs

To generate rabbit anti-human endosialin (TEM1) antibodies 1-3-1, 1-25-2 and 1-55-2, we prepared a soluble human endosialin extracellular domain-mouse Fc fusion protein as follows. We cloned the Fc fragment from the anti-MORab-003 mouse monoclonal antibody (Ebel et al. Cancer Immun. 2007. 7:6-13) by RT-PCR. The 5′ primer (1443: CAGTGTTAACGACGACGACGACAAAGAGCCCAGCGGACCAATTTCAACAATC AACCCC (SEQ ID NO: 258)) used for this cloning contained an HpaI site and an enterkinase cleavage site. The 3′ primer (1451: GGGTTCGAATCATTTACCCGGAGACCGGGAGATGG (SEQ ID NO: 259)) contained a BstBI site. The RT-PCR product was cloned into pEF6 (Invitrogen, Carlsbad, Calif.) at the HpaI/BstBI sites, producing pEF6-EK-IgG2b. Then, we cloned the extracellular domain of hTEM1 in-frame into the EcoRI/HpaI sites of pEF6-EK-IgG2b. The construct was transfected into CHO-K1 cells and selected with 5 μg/mL blasticidin. Secreted TEM1-Fc was purified on a protein A column using standard methods and the purified product used for rabbit immunization.

We immunized two New Zealand white rabbits with four injections of purified secreted TEM1-Fc (300 μg in the initial injection, 200 μg per booster injection). Antiserum titer was monitored using standard ELISA (TEM1) and counterscreen ELISA for Fc reactivity. A final IV boost was performed within 4-8 weeks of the last regular injection boost. Splenectomies were performed after the final IV boost. Lymphocytes were isolated from each rabbit spleen. Fusions with 240E-W3 cells (Epitomics) performed in forty 96-well plates. Standard ELISA screening for 40 plates was performed using TEM1-Fc protein. All positive hybridomas were expanded to 24-well plates and confirmatory standard and differential ELISA were performed. Fc reactive clones were counterscreened and removed, leaving the TEM1-reactive clones. Positive hybridomas were expanded for Ig gene sequencing and generation of purified Ab. Three positive hybridomas were assigned the designations 1-3-1, 1-25-2 and 1-55-2.

### Example 2

### Generation of Rabbit Anti-mEndosialin MAbs

To generate rabbit anti-mouse endosialin (TEM1) antibody clone 8, we immunized two New Zealand white rabbits with four injections of mouse TEM1 extracellular domain (ECD)-Fc fusion protein. Antiserum titer was monitored using standard ELISA (TEM1) and counterscreen ELISA for Fc reactivity. A final IV boost was performed within 4-8 weeks of the last regular injection boost. Splenectomies were performed after the final IV boost. Lymphocytes were isolated from each rabbit spleen. Fusions with 240E-W3 cells (Epitomics) were performed in forty 96-well plates. Standard ELISA screening for 40 plates was performed using TEM1-Fc protein. All positive hybridomas were expanded to 24-well plates and confirmatory standard and differential ELISA were performed. Fc reactive clones were counterscreened and removed, leaving the TEM1-reactive clones. Positive hybridomas were expanded for Ig gene sequencing and generation of purified Ab. One positive hybridoma was assigned the designation clone 8.

### Example 3

### Cloning and Sequencing of Rabbit IgG Anti-TEM1 Antibodies

To clone the variable regions of the rabbit anti-endosialin/TEM1 monoclonal antibodies, we isolated total RNA from murine hybridomas using RNAQUEOUS™ (Ambion) according to the manufacturer's instructions. We synthesized cDNA using SUPERSCRIPT III™ reverse transcriptase (Invitrogen) according to the manufacturer's instructions.

To amplify the variable region of the light chain, we carried out PCR reactions with TAQPRO™ DNA polymerase (Denville) using a 100 mixture of primers 1858 to 1863 (see Table 2). To amplify the variable regions of the heavy chains, we carried out the PCR reactions with the TAQPRO™ DNA polymerase (Denville) using a 100 mixture of primers 1852 to 1856 (see Table 2).

We cloned the PCR products into pCR4-TOPO vector (Invitrogen), transformed into E. coli Mach1 cells and selected transformants on LB Kanamycin plates. We screened colonies for inserts with flanking plasmid insert primers 390 and 391 (see Table 2) and used positive colonies to generate template miniprep DNA for DNA sequence determination. We sequenced DNA inserts with Morphotek primers 390 and 391 using Beckman Coulter DTCS sequencing reagent followed by data acquisition and analysis on a Beckman Coulter CEQ2000 (with CEQ3000 software).

TABLE 2
PRIMERS USED FOR PCR AND SEQUENCING

| Primer | |
|---|---|
| # | Primer Sequence |
| 390 | CCCAGTCACGACGTTGTAAAACG |
| | (SEQ ID NO: 134) |
| 391 | AGCGGATAACAATTTCACACAGG |
| | (SEQ ID NO: 135) |
| 1212 | GTCATCTAGACACGCTGGTTCTGCAGGTCTGCA |
| | (SEQ ID NO: 256) |
| 1213 | GATCGAATTCACCATGCTGCTGCGCCTGTTGCTGG |
| | (SEQ ID NO: 257) |
| 1443 | CAGTGTTAACGACGACGACGACAAAGAGCCCAGCG |
| | GACCAATTTCAACAATCAACCCC |
| | (SEQ ID NO: 258) |
| 1451 | GGGTTCGAATCATTTACCCGGAGACCGGGAGATGG |
| | (SEQ ID NO: 259) |
| 1852 | GCTGCCCAACCAGCCATGGCCCAGTCGGTGGAGGA |
| | GTCCRGG |
| | (SEQ ID NO: 136) |
| 1853 | GCTGCCCAACCAGCCATGGCCCAGTCGGTGAAGGA |
| | GTCCGAG |
| | (SEQ ID NO: 137) |
| 1854 | GCTGCCCAACCAGCCATGGCCCAGTCGYTGGAGGA |
| | GTCCGGG |
| | (SEQ ID NO: 138) |
| 1855 | GCTGCCCAACCAGCCATGGCCCAGSAGCAGCTGRTG |
| | GAGTCCGG |
| | (SEQ ID NO: 139) |
| 1856 | TGARGAGAYGGTGACCAGGGTGCC |
| | (SEQ ID NO: 140) |
| 1858 | GGGCCCAGGCGGCCGAGCTCGTGMTGACCCAGACTC |
| | CA |
| | (SEQ ID NO: 141) |
| 1859 | GGGCCCAGGCGGCCGAGCTCGATMTGACCCAGACTC |
| | CA |
| | (SEQ ID NO: 142) |
| 1860 | GGGCCCAGGCGGCCGAGCTCGTGATGACCCAGACTG |
| | AA |
| | (SEQ ID NO: 143) |
| 1861 | TAGGATCTCCAGCTCGGTCCC |
| | (SEQ ID NO: 144) |
| 1862 | TTTTGATTTCCACATTGGTGCC |
| | (SEQ ID NO: 145) |
| 1863 | TTTTGACSACCACCTCGGTCCC |
| | (SEQ ID NO: 146) |

### Example 4

### Generation of A431-TEM1 Cell Line

We used A431 cells (ATCC CRL-1555), a human epidermoid cancer cell line, to prepare a cell line for studying the effect of TEM1 expression on in vivo tumor growth. Briefly, we prepared p246 (pEF6-hTEM1) containing full length human TEM1 as follows. We amplified full length hTEM1 with a 5′ primer containing an EcoRI site and a Kozak initiation site (1213: GATCGAATTCACCATGCTGCTGCGCCTGTTGCTGG (SEQ ID NO: 257)) along with a 3′ primer homologous to the end of hTEM1 without a stop codon but containing an XbaI site (1212: GTCATCTAGACACGCTGGTTCTGCAGGTCTGCA (SEQ ID NO: 256)). This fragment was digested with EcoRI/XbaI and cloned into pEF6 digested with EcoRI/XbaI yielding full length hTEM1 with a C-terminal V5-His tag. We isolated p246 (pEF6-hTEM1) DNA with a MaxiPrep kit (Qiagen) and linearized fifty micrograms of plasmid with 70 units of ScaI restriction endonuclease (New England BioLabs, Ipswich, Mass.) for 1.5 hours at 37° C. We extracted the DNA with phenol/chloroform and precipitated with 2 volumes cold Ethanol. We resuspended the DNA in sterile dH2O and quantitated.

We deadhered adherent A431 cells for 2 min at room temperature with 5 mL of TRYPLE™ cell dissociation enzyme (Gibco). After recovering the cells, TRYPLE™ was inactivated with 5 mL 10% complete DMEM. We pelleted the cells and washed twice with 10 mL ice cold PBS (Gibco), counted, and resuspended at 5.8×10⁶ cells/mL in plain ice cold RPMI 1640 (Gibco). We placed 300 μL of the cell suspension in a 0.4 cm gap cuvette with 10 ug of linearized DNA and electroporated at 260V/1,000 μF. Cells were placed in 5 mL complete DMEM in a T-25 flask at 37° C./5%/CO₂ overnight. The following day, blasticidin was added to the media at 5 μg/mL and cells were selected for stable integration of the plasmid.

To enrich for cells expressing TEM1 on the surface, blasticidin resistant cells were harvested with enzyme-free dissociation media (Gibco) and washed as before. Cells were resuspended in 1 mL facs buffer (PBS/2% FBS) containing 20 μg/mL MORAb-004 antibody (U.S. Patent Application Publication Nos. 2006/0239911 and 2008/0248034; Tomkowicz et al. (2007) Proc. Natl. Acad. Sci. USA 104: 17965-17970) and incubated one hour on ice. Cells were washed twice with 5 mL facs buffer, and resuspended in 1 mL facs buffer containing 5 uL (1:1000 dilution) goat anti-human IgG (H+L) FITC (Jackson Immuno) for 30 minutes on ice. Cells were washed twice with 5 mL facs buffer, and resuspended in 1 mL facs buffer containing 5 μL VIAPROBE™ (Becton Dickinson). Cells were gated to sort 2.5E4 events of high TEM1 expressing cells. These were then expanded and frozen in liquid nitrogen until needed.

### Example 5

### Production of TEM-1 Expressing Tumors In Vivo

To investigate the effect of surface expression of TEM1 on tumor growth, we generated xenograft tumors in nude mice. Briefly, 5×10⁶ A431 or A431-TEM1 cells were injected into athymic NCr-nu/nu nude mice subcutaneously to induce tumor formation at Southern Research Institute. Following injection, tumors were allowed to grow and were harvested on day 15.

Frozen and formalin-fixed, paraffin embedded (FFPE) tumor blocks for IHC testing were prepared as follows. When tumors reached ˜2 grams, A431 and A431-TEM1 tumors were flash frozen in OTC. In parallel, A431 and A431-TEM1 tumors were formalin fixed and embedded in paraffin. All samples were prepared at Charles River.

### Example 6

### Immunohistochemistry (IHC) Assay

For the immunohistochemical staining with rabbit anti-endosialin monoclonal antibodies of the invention, we used an indirect method utilizing a Dako ENVISION™+, HRP (rabbit) kit.

Formalin fixed paraffin embedded tissue sections were deparaffinized and run through deionized water and rinsed 2× with PBST (phosphate buffered saline [0.15M NaCl, pH 72]) with 0.05% TWEEN 20™. Peroxidase solution (supplied in Dako Envision kit) was applied for 5 minutes followed by rinsing 2× with PBST. Next, a serum block was applied for 20 minutes. The serum block was composed of PBS (0.15M NaCl, pH 7.2); 0.5% casein; 1% bovine serum albumin; 5% human gamma globulins, and 1 mg/mL of heat aggregated human IgG. Next, the primary antibody (5.0±0.5 mg/mL antibody in 10 mM sodium phosphate, 150 mM sodium chloride, pH 7.2, 0.01% USP Tween 80) was added for 1 hour followed by rinsing 2× with PBST. The peroxidase labeled polymer (supplied in Dako ENVISION™ kit) was then applied for 30 minutes followed by rinsing 2× with PBST. Next, the substrate-chromogen solution (supplied in Dako ENVISION™ kit) was applied for 8 minutes followed by rinsing thoroughly in tap water. All slides were rinsed with tap water, counterstained with hematoxylin, washed, blued in saturated lithium carbonate, washed, dehydrated through alcohols, cleared in xylene, and coverslipped for interpretation.

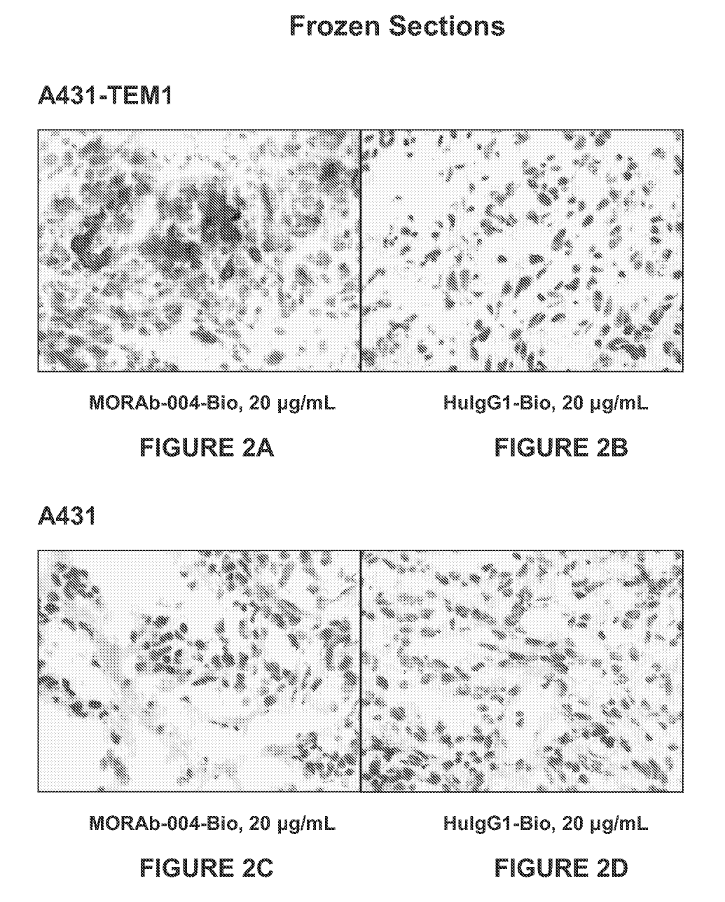

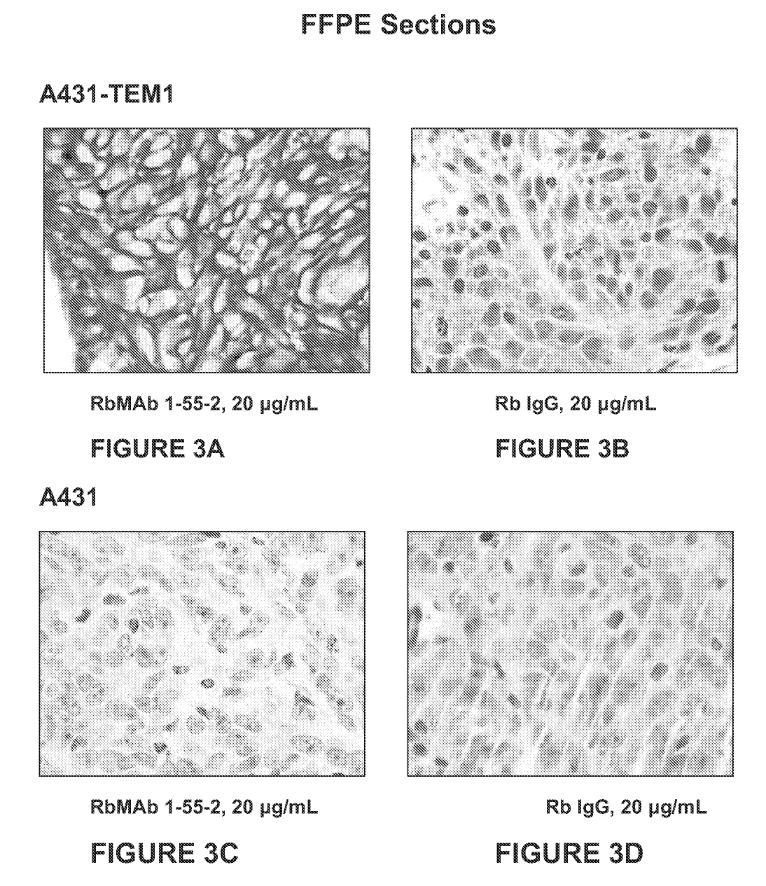

All rabbit anti-endosialin antibodies (FIG. 3) tested and MORAb-004, a humanized anti-hTEM1 monoclonal antibody (FIG. 2), stained FFPE samples from a TEM1-expressing tumor but not from the control (A431) tumor.

### Example 7

### Inhibition of Endosialin/TEM1 Binding to Fibronectin

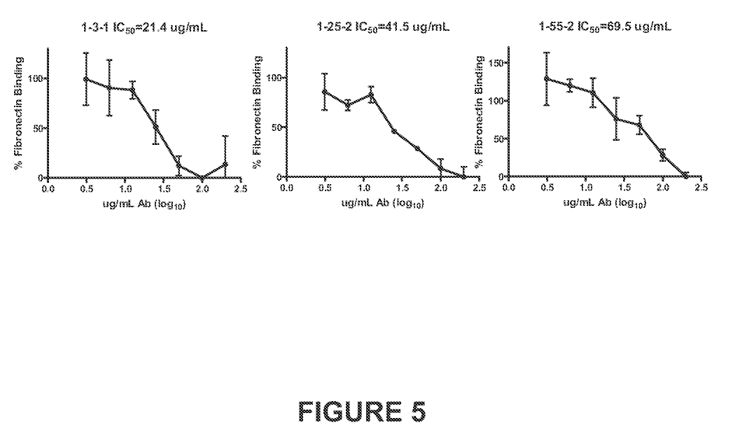

Recombinant hTEM1-Fc protein was dissolved in assay buffer at 15 μg/mL. Anti-hTEM1 rabbit antibodies were added to the hTEM1-Fc protein at two-fold serial dilutions starting at 200 μg/mL. Plates were incubated for 1.5 h at room temperature. Complexes were then added to FN-coated plates and incubated 1.5 h at room temperature. The plate was washed three times with 200 μL PBS-T and 100 μL HRP-conjugated goat-anti-mouse Ab (1:1000 dilution in assay buffer) was added for 1 h at room temperature. Plate was washed three times with 200 μL PBS-T and 100 μL SureBlue Substrate added for 10 minutes at room temperature. Reaction was stopped by the addition of 50 μL H₂SO₄ and the A₄₅₀ quantitated. The dose/response data and IC₅₀ are shown in FIG. 5.

### Example 8

### Humanization of the Rabbit Anti-Endosialin MAbs

To generate humanized anti-human endosialin (TEM1) antibodies Hu 1-3-1, Hu 1-25-2 and Hu 1-55-2, the primary amino acid sequences of the variable Hc and Lc were compared to the human germline variable domains using IgBlast (National Center for Biotechnology Information). The closest human homologues were used for the backbones of the humanized variable regions. We then grafted the rabbit CDRs in silico into these backbones and added the corresponding human constant regions of the light and heavy chains to the 3′ ends. The constructs were then codon optimized and synthesized by GenScript (Piscataway, N.J.). The constructs were cloned into the corresponding glutamine synthetase (GS) vectors (Lonza) for cell line development using standard protocols. The SEQ ID NOs corresponding to the full length heavy and light chain amino acid and nucleotide sequences of the humanized anti-endosialin antibodies are found in Table 3.

TABLE 3
FULL LENGTH HEAVY AND LIGHT CHAIN
SEQUENCE IDENTIFIERS (SEQ ID NOs)

| | | Hu 1-3-1 | Hu 1-25-2 | Hu 1-55-2 |
|---|---|---|---|---|
| HEAVY CHAIN | PROT | 156 | 185 | 189 |
| | DNA | 147 | 184 | 188 |
| LIGHT CHAIN | PROT | 174 | 187 | 191 |
| | DNA | 165 | 186 | 190 |

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well known and commonly used in the art.

All publications, patents, patent applications or other documents cited herein are hereby incorporated by reference in their entirety for all purposes to the same extent as if each individual publication, patent, patent application, or other document was individually indicated to be incorporated by reference for all purposes.

Throughout this specification and claims, the word “comprise,” or variations such as “comprises” or “comprising,” will be understood to imply the inclusion of a stated integer or group of integers but not the exclusion of any other integer or group of integers.

TABLE 4
SEQUENCE TABLE

HUMAN ENDOSIALIN DNA
Underline: extracellular domain.

(SEQ ID NO: 1)

atgctgctgcgcctgttgctggcctgggcggccgcagggcccacactg
ggccaggacccctgggctgctgagccccgtgccgcctgcggccccagc
agctgctacgctctcttcccacggcgccgcaccttcctggaggcctgg
cgggcctgccgcgagctggggggcgacctggccactcctcggaccccc
gaggaggcccagcgtgtggacagcctggtgggtgcgggcccagccagc
cggctgctgtggatcgggctgcagcggcaggcccggcaatgccagctg
cagcgcccactgcgcggcttcacgtggaccacaggggaccaggacacg
gctttcaccaactgggcccagccagcctctggaggcccctgcccggcc
cagcgctgtgtggccctggaggcaagtggcgagcaccgctggctggag
ggctcgtgcacgctggctgtcgacggctacctgtgccagtttggcttc
gagggcgcctgcccggcgctgcaagatgaggcgggccaggccggccca
gccgtgtataccacgcccttccacctggtctccacagagtttgagtgg
ctgcccttcggctctgtggccgctgtgcagtgccaggctggcagggga
gcctctctgctctgcgtgaagcagcctgagggaggtgtgggctggtca
cgggctgggcccctgtgcctggggactggctgcagccctgacaacggg
ggctgcgaacacgaatgtgtggaggaggtggatggtcacgtgtcctgc
cgctgcactgagggcttccggctggcagcagacgggcgcagttgcgag
gacccctgtgcccaggctccgtgcgagcagcagtgtgagcccggtggg
ccacaaggctacagctgccactgtcgcctgggtttccggccagcggag
gatgatccgcaccgctgtgtggacacagatgagtgccagattgccggt
gtgtgccagcagatgtgtgtcaactacgttggtggcttcgagtgttat
tgtagcgagggacatgagctggaggctgatggcatcagctgcagccct
gcaggggccatgggtgcccaggcttcccaggacctcggagatgagttg
ctggatgacggggaggatgaggaagatgaagacgaggcctggaaggcc
ttcaacggtggctggacggagatgcctgggatcctgtggatggagcct
acgcagccgcctgactttgccctggcctatagaccgagcttcccagag
gacagagagccacagataccctacccggagcccacctggccacccccg
ctcagtgcccccagggtcccctaccactcctcagtgctctccgtcacc
cggcctgtggtggtctctgccacgcatcccacactgccttctgcccac
cagcctcctgtgatccctgccacacacccagctttgtcccgtgaccac
cagatccccgtgatcgcagccaactatccagatctgccttctgcctac
caacccggtattctctctgtctctcattcagcacagcctcctgcccac
cagccccctatgatctcaaccaaatatccggagctcttccctgcccac
cagtcccccatgtttccagacacccgggtcgctggcacccagaccacc
actcatttgcctggaatcccacctaaccatgcccctctggtcaccacc
ctcggtgcccagctaccccctcaagccccagatgcccttgtcctcaga
acccaggccacccagcttcccattatcccaactgcccagccctctctg
accaccacctccaggtcccctgtgtctcctgcccatcaaatctctgtg
cctgctgccacccagcccgcagccctccccaccctcctgccctctcag
agccccactaaccagacctcacccatcagccctacacatccccattcc
aaagccccccaaatcccaagggaagatggccccagtcccaagttggcc
ctgtggctgccctcaccagctcccacagcagccccaacagccctgggg
gaggctggtcttgccgagcacagccagagggatgaccggtggctgct
ggtggcactcctggtgccaacgtgtgtctttttggtggtcctgcttgc
actgggcatcgtgtactgcacccgctgtggcccccatgcacccaacaa
gcgcatcactgactgctatcgctgggtcatccatgctgggagcaagag
cccaacagaacccatgccccccaggggcagcctcacaggggtgcagac
ctgcagaaccagcgtgtga
HUMAN ENDOSIALIN PROTEIN

(SEQ ID NO: 2)

MLLRLLLAWAAAGPTLGQDPWAAEPRAACGPSSCYALFPRRRTFLEAW
RACRELGGDLATPRTPEEAQRVDSLVGAGPASRLLWIGLQRQARQCQL
QRPLRGFTWTTGDQDTAFTNWAQPASGGPCPAQRCVALEASGEHRWLE
GSCTLAVDGYLCQFGFEGACPALQDEAGQAGPAVYTTPFHLVSTEFEW
LPFGSVAAVQCQAGRGASLLCVKQPEGGVGWSRAGPLCLGTGCSPDNG
GCEHECVEEVDGHVSCRCTEGFRLAADGRSCEDPCAQAPCEQQCEPGG
PQGYSCHCRLGFRPAEDDPHRCVDTDECQIAGVCQQMCVNYVGGFECY
CSEGHELEADGISCSPAGAMGAQASQDLGDELLDDGEDEEDEDEAWKA
FNGGWTEMPGILWMEPTQPPDFALAYRPSFPEDREPQIPYPEPTWPPP
LSAPRVPYHSSVLSVTRPVVVSATHPTLPSAHQPPVIPATHPALSRDH
QIPVIAANYPDLPSAYQPGILSVSHSAQPPAHQPPMISTKYPELFPAH
QSPMFPDTRVAGTQTTTHLPGIPPNHAPLVTTLGAQLPPQAPDALVLR
TQATQLPIIPTAQPSLTTTSRSPVSPAHQISVPAATQPAALPTLLPSQ
SPTNQTSPISPTHPHSKAPQIPREDGPSPKLALWLPSPAPTAAPTALG
EAGLAEHSQRDDRWLLVALLVPTCVFLVVLLALGIVYCTRCGPHAPNK
RITDCYRWVIHAGSKSPTEPMPPRGSLTGVQTCRTSV
MOUSE ENDOSIALIN DNA

(SEQ ID NO: 3)

atgctgctgcgcctgctgctggcctgggtggccgcggtgcccgcactg
ggccaggtcccctggacgccggagcctcgagccgcgtgcggccccagc
agctgctacgcgctctttccccggcgccgcacattcctggaagcttgg
cgggcgtgccgcgaattggggggcaacctggccacaccgcggacccca
gaggaggcccagcgtgtggacagcctggtgggggtcgggccggccaac
gggctgctatggattgggttgcagcggcaggctaggcaatgccagccg
cagcgcccactgcggggcttcatatggaccacgggagaccaggacacc
gccttcaccaactgggcccagccggctacggaaggaccctgcccagcc
cagcgctgtgcagcccttgaggccagcggagagcatcgctggctcgaa
ggctcgtgcacactggctgtcgatggctacctctgccagtttggtttt
gagggtgcctgccctgccttgccgcttgaggtgggtcaggccggtccc
gctgtctacaccacacccttcaacctggtttccagcgagttcgaatgg
ctgccctttggctccgtggcagctgtgcagtgccaagctggcagggga
gcttctctgctgtgcgtgaaacagccttcaggtggcgtgggctggtcc
cagactggcccgctgtgcccagggactggctgtggtcctgacaatggg
ggttgcgaacatgagtgtgtggaagaggtggacggtgctgtgtcctgc
cgctgcagtgaaggcttccgtctagcagcagatgggcacagttgtgaa
gacccctgtgcccaggccccctgtgagcagcagtgtgaacctggaggg
ccacaaggctatagctgccactgtcgccttggcttccggccagctgag
gatgatccacaccgctgcgtggacacggatgagtgccagattgctggt
gtgtgccagcagatgtgtgtcaactatgttggtggctttgagtgttac
tgcagcgagggtcacgagcttgaggcagatggtatcagctgtagccct
gcaggagccatgggtgcccaggcttcccaggatctcagagatgagttg
ctggatgatggagaagaaggggaggatgaagaggagccctgggaggac
tttgatggcacctggacagaggaacaggggatcctatggctggcacct
acacatccacctgactttggcctgccctataggcccaacttcccacag
gatggagagcctcagagattgcacctggagcctacctggccaccccca
cttagtgcccccaggggcccctaccactcctcagtggtgtctgccaca
cggcccatggtgatctctgccactcgacccacactaccttctgcccac
aagacctctgttatttcagctacacgcccacccctgagccctgtccac
ccacctgccatggcccctgccacacctccagctgtgttctctgagcac
cagatccccaaaatcaaggccaattatccagacctgccttttggccac
aagcctgggataacctcggccactcacccagcacggtctcctccgtac
cagccccccattatctcaaccaactatccccaagtcttccctccccac
caggcccctatgtctccagatacccacactatcacttatttgcctcca
gtcccccctcaccttgatcctggggataccacttctaaagcccatcaa
caccctttgctcccagatgctccaggtatcagaacccaggccccccag
ctttctgtctcagctctccagccccctcttcctaccaactccaggtct
tctgtccatgaaactcctgtgcctgctgccaaccagcccccagccttc
ccttcttctcccctcccccctcagaggcccactaaccagacctcatct
atcagccctacacattcctattccagagcccctctagtcccaagggaa
ggagttcccagtcccaaatcagtgccacagctgccctcggtgccctcc
acagcagctccaacagccctggcagagtcaggtcttgcaggccaaagc
caaagggatgaccgctggctgctggtggcactcctggtgccaacatgt
gtcttcttggtggtgctgcttgccctgggcattgtgtactgcactcgc
tgtggctcccacgcacccaacaagcggatcacggactgctatcgctgg
gtcacacatgctgggaacaagagctcaacagaacccatgccccccaga
ggcagccttacaggggtacagacctgtagaaccagtgtgtga
MOUSE ENDOSIALIN PROTEIN

(SEQ ID NO: 4)

MLLRLLLAWVAAVPALGQVPWTPEPRAACGPSSCYALFPRRRTFLEAW
RACRELGGNLATPRTPEEAQRVDSLVGVGPANGLLWIGLQRQARQCQP
QRPLRGFIWTTGDQDTAFTNWAQPATEGPCPAQRCAALEASGEHRWLE
GSCTLAVDGYLCQFGFEGACPALPLEVGQAGPAVYTTPFNLVSSEFEW
LPFGSVAAVQCQAGRGASLLCVKQPSGGVGWSQTGPLCPGTGCGPDNG
GCEHECVEEVDGAVSCRCSEGFRLAADGHSCEDPCAQAPCEQQCEPGG
PQGYSCHCRLGFRPAEDDPHRCVDTDECQIAGVCQQMCVNYVGGFECY
CSEGHELEADGISCSPAGAMGAQASQDLRDELLDDGEEGEDEEEPWED
FDGTWTEEQGILWLAPTHPPDFGLPYRPNFPQDGEPQRLHLEPTWPPP
LSAPRGPYHSSVVSATRPMVISATRPTLPSAHKTSVISATRPPLSPVH
PPAMAPATPPAVFSEHQIPKIKANYPDLPFGHKPGITSATHPARSPPY
QPPIISTNYPQVFPPHQAPMSPDTHTITYLPPVPPHLDPGDTTSKAHQ
HPLLPDAPGIRTQAPQLSVSALQPPLPTNSRSSVHETPVPAANQPPAF
PSSPLPPQRPTNQTSSISPTHSYSRAPLVPREGVPSPKSVPQLPSVPS
TAAPTALAESGLAGQSQRDDRWLLVALLVPTCVFLVVLLALGIVYCTR
CGSHAPNKRITDCYRWVTHAGNKSSTEPMPPRGSLTGVQTCRTSV
ENDOSIALIN SEQUENCE FROM PLASMID pEF6-hTEM1

(SEQ ID NO: 5)

ATGCTGCTGCGCCTGTTGCTGGCCTGGGCGGCCGCAGGGCCCACACTG
GGCCAGGACCCCTGGGCTGCTGAGCCCCGTGCCGCCTGCGGCCCCAGC
AGCTGCTACGCTCTCTTCCCACGGCGCCGCACCTTCCTGGAGGCCTGG
CGGGCCTGCCGCGAGCTGGGGGGCGACCTGGCCACTCCTCGGACCCCC
GAGGAGGCCCAGCGTGTGGACAGCCTGGTGGGTGCGGGCCCAGCCAGC
CGGCTGCTGTGGATCGGGCTGCAGCGGCAGGCCCGGCAATGCCAGCTG
CAGCGCCCACTGCGCGGCTTCACGTGGACCACAGGGGACCAGGACACG
GCTTTCACCAACTGGGCCCAGCCAGCCTCTGGAGGCCCCTGCCCGGCC
CAGCGCTGTGTGGCCCTGGAGGCAAGTGGCGAGCACCGCTGGCTGGAG
GGCTCGTGCACGCTGGCTGTCGACGGCTACCTGTGCCAGTTTGGCTTC
GAGGGCGCCTGCCCGGCGCTGCAAGATGAGGCGGGCCAGGCCGGCCCA
GCCGTGTATACCACGCCCTTCCACCTGGTCTCCACAGAGTTTGAGTGG
CTGCCCTTCGGCTCTGTGGCCGCTGTGCAGTGCCAGGCTGGCAGGGGA
GCCTCTCTGCTCTGCGTGAAGCAGCCTGAGGGAGGTGTGGGCTGGTCA
CGGGCTGGGCCCCTGTGCCTGGGGACTGGCTGCAGCCCTGACAACGGG
GGCTGCGAACACGAATGTGTGGAGGAGGTGGATGGTCACGTGTCCTGC
CGCTGCACTGAGGGCTTCCGGCTGGCAGCAGACGGGCGCAGTTGCGAG
GACCCCTGTGCCCAGGCTCCGTGCGAGCAGCAGTGTGAGCCCGGTGGG
CCACAAGGCTACAGCTGCCACTGTCGCCTGGGTTTCCGGCCAGCGGAG
GATGATCCGCACCGCTGTGTGGACACAGATGAGTGCCAGATTGCCGGT
GTGTGCCAGCAGATGTGTGTCAACTACGTTGGTGGCTTCGAGTGTTAT
TGTAGCGAGGGACATGAGCTGGAGGCTGATGGCATCAGCTGCAGCCCT
GCAGGGGCCATGGGTGCCCAGGCTTCCCAGGACCTCGGAGATGAGTTG
CTGGATGACGGGGAGGATGAGGAAGATGAAGACGAGGCCTGGAAGGCC
TTCAACGGTGGCTGGACGGAGATGCCTGGGATCCTGTGGATGGAGCCT
ACGCAGCCGCCTGACTTTGCCCTGGCCTATAGACCGAGCTTCCCAGAG
GACAGAGAGCCACAGATACCCTACCCGGAGCCCACCTGGCCACCCCCG
CTCAGTGCCCCCAGGGTCCCCTACCACTCCTCAGTGCTCTCCGTCACC
CGGCCTGTGGTGGTCTCTGCCACGCATCCCACACTGCCTTCTGCCCAC
CAGCCTCCTGTGATCCCTGCCACACACCCAGCTTTGTCCCGTGACCAC
CAGATCCCCGTGATCGCAGCCAACTATCCAGATCTGCCTTCTGCCTAC
CAACCCGGTATTCTCTCTGTCTCTCATTCAGCACAGCCTCCTGCCCAC
CAGCCCCCTATGATCTCAACCAAATATCCGGAGCTCTTCCCTGCCCAC
CAGTCCCCCATGTTTCCAGACACCCGGGTCGCTGGCACCCAGACCACC
ACTCATTTGCCTGGAATCCCACCTAACCATGCCCCTCTGGTCACCACC
CTCGGTGCCCAGCTACCCCCTCAAGCCCCAGATGCCCTTGTCCTCAGA
ACCCAGGCCACCCAGCTTCCCATTATCCCAACTGCCCAGCCCTCTCTG
ACCACCACCTCCAGGTCCCCTGTGTCTCCTGCCCATCAAATCTCTGTG
CCTGCTGCCACCCAGCCCGCAGCCCTCCCCACCCTCCTGCCCTCTCAG
AGCCCCACTAACCAGACCTCACCCATCAGCCCTACACATCCCCATTCC
AAAGCCCCCCAAATCCCAAGGGAAGATGGCCCCAGTCCCAAGTTGGCC
CTGTGGCTGCCCTCACCAGCTCCCACAGCAGCCCCAACAGCCCTGGGG
GAGGCTGGTCTTGCCGAGCACAGCCAGAGGGATGACCGGTGGCTGCTG
GTGGCACTCCTGGTGCCAACGTGTGTCTTTTTGGTGGTCCTGCTTGCA
CTGGGCATCGTGTACTGCACCCGCTGTGGCCCCCATGCACCCAACAAG
CGCATCACTGACTGCTATCGCTGGGTCATCCATGCTGGGAGCAAGAGC
CCAACAGAACCCATGCCCCCCAGGGGCAGCCTCACAGGGGTGCAGACC
TGCAGAACCAGCGTGTCTAGAGGGCCCTTCGAAGGTAAGCCTATCCCT
AACCCTCTCCTCGGTCTCGATTCTACGCGTACCGGTCATCATCACCAT
CACCATTGA
1-3-1 Heavy Chain Variable Region
NUCLEOTIDE SEQUENCE:

(SEQ ID NO: 6)

GCCCAGTCGGTGGAGGAGTCCGGGGGAGACCTGGTCAGGCCGGGGGCG
TCCCTGACACTCACCTGCTCAGCTTCTGGATTCACCATCAATAGGAAC
TACTGGATATGCTGGGTCCGCCAGGCTCCAGGGAGGGGGCCGGAGTGG
ATCGCTTGTATTTATGGTGGTAGTAGTGGTACCACTTACTACACGAAC
TGGGCGAAAGGCCGGTTCACCATCTCCAAAGCCGCGTCGACCACGGTG
ACTCTGCAAATGACCAGTCTGACAGTCGCGGACACGGCCACCTATTTC
TGTGCGAGAGTTGATAATGGTGGTGATTGGTATTTCAGGTTGTGGGGC
CCAGGCACCCTGGTCACCATCTCCTCA
1-3-1 Heavy Chain FW1:

(SEQ ID NO: 7)

GCCCAGTCGGTGGAGGAGTCCGGGGGAGACCTGGTCAGGCCGGGGGCG
TCCCTGACACTCACCTGC
1-3-1 Heavy Chain CDR1:

(SEQ ID NO: 8)

TCAGCTTCTGGATTCACCATCAATAGGAACTAC
1-3-1 Heavy Chain FW2:

(SEQ ID NO: 9)

TGGATATGCTGGGTCCGCCAGGCTCCAGGGAGGGGGCCGGAGTGGATC
GCT
1-3-1 Heavy Chain CDR2:

(SEQ ID NO: 10)

TGTATTTATGGTGGTAGTAGTGGTACCACTTACTACACGAACTGGGCG
AAAGGC
1-3-1 Heavy Chain FW3:

(SEQ ID NO: 11)

CGGTTCACCATCTCCAAAGCCGCGTCGACCACGGTGACTCTGCAAATG
ACCAGTCTGACAGTCGCGGACACGGCCACCTATTTCTGTGCGAGA
1-3-1 Heavy Chain CDR3:

(SEQ ID NO: 12)

GTTGATAATGGTGGTGATTGGTATTTCAGGTTG
1-3-1 Heavy Chain FW4:

(SEQ ID NO: 13)

TGGGGCCCAGGCACCCTGGTCACCATCTCCTCA
PROTEIN SEQUENCE

(SEQ ID NO: 14)

AQSVEESGGDLVRPGASLTLTCSASGFTINRNYWICWVRQAPGRGPEW
IACIYGGSSGTTYYTNWAKGRFTISKAASTTVTLQMTSLTVADTATYF
CARVDNGGDWYFRLWGPGTLVTISS
1-3-1 Heavy Chain FW1:

(SEQ ID NO: 15)

AQSVEESGGDLVRPGASLTLTC
1-3-1 Heavy Chain CDR1:

(SEQ ID NO: 16)

SASGFTINRNY
1-3-1 Heavy Chain FW2:

(SEQ ID NO: 17)

WICWVRQAPGRGPEWIA
1-3-1 Heavy Chain CDR2:

(SEQ ID NO: 18)

CIYGGSSGTTYYTNWAKG
1-3-1 Heavy Chain FW3:

(SEQ ID NO: 19)

RFTISKAASTTVTLQMTSLTVADTATYFCAR
1-3-1 Heavy Chain CDR3:

(SEQ ID NO: 20)

VDNGGDWYFRL
1-3-1 Heavy Chain FW4:

(SEQ ID NO: 21)

WGPGTLVTISS
1-3-1 Kappa Chain Variable Region
NUCLEOTIDE SEQUENCE:

(SEQ ID NO: 22)

GAGCTCGATCTGACCCAGACTCCAGCCTCTGTGGAGGTAGCTGTGGGA
GGCACAGTCACCATCAAGTGCCAGGCCAGTCAGAGCATTAGTAGCTAC
TTAGCCTGGTATCAGCAGAAACCAGGACAGCCTCCCAAGCTCCTGATC
TACAGGGCATCCACGCTGGCATCTGGGGTCTCATCGCGGTTCAAAGGC
AGTGGATCTGGGACAGAGTTCACTCTCACCATCAGCGGTGTGCAGTGT
GACGATGCTGCCACTTACTACTGTCAACAGGGTGTGCGTGTGGTTGAT
ATTGATAATTCTTTCGGCGGAGGGACCGAACTGGTGGTCAAA
1-3-1 Kappa Chain FW1:

(SEQ ID NO: 23)

GAGCTCGATCTGACCCAGACTCCAGCCTCTGTGGAGGTAGCTGTGGGA
GGCACAGTCACCATCAAGTGC
1-3-1 Kappa Chain CDR1:

(SEQ ID NO: 24)

CAGGCCAGTCAGAGCATTAGTAGCTACTTAGCC
1-3-1 Kappa Chain FW2:

(SEQ ID NO: 25)

TGGTATCAGCAGAAACCAGGACAGCCTCCCAAGCTCCTGATCTAC
1-3-1 Kappa Chain CDR2:

(SEQ ID NO: 26)

AGGGCATCCACGCTGGCATCT
1-3-1 Kappa Chain FW3:

(SEQ ID NO: 27)

GGGGTCTCATCGCGGTTCAAAGGCAGTGGATCTGGGACAGAGTTCACT
CTCACCATCAGCGGTGTGCAGTGTGACGATGCTGCCACTTACTACTGT
1-3-1 Kappa Chain CDR3:

(SEQ ID NO: 28)

CAACAGGGTGTGCGTGTGGTTGATATTGATAATTCT
1-3-1 Kappa Chain FW4:

(SEQ ID NO: 29)

TTCGGCGGAGGGACCGAACTGGTGGTCAAA
PROTEIN SEQUENCE

(SEQ ID NO: 30)

ELDLTQTPASVEVAVGGTVTIKCQASQSISSYLAWYQQKPGQPPKLLI
YRASTLASGVSSRFKGSGSGTEFTLTISGVQCDDAATYYCQQGVRVVD
IDNSFGGGTELVVK
1-3-1 Kappa Chain FW1:

(SEQ ID NO: 31)

ELDLTQTPASVEVAVGGTVTIKC
1-3-1 Kappa Chain CDR1:

(SEQ ID NO: 32)

QASQSISSYLA
1-3-1 Kappa Chain FW2:

(SEQ ID NO: 33)

WYQQKPGQPPKLLIY
1-3-1 Kappa Chain CDR2:

(SEQ ID NO: 34)

RASTLAS
1-3-1 Kappa Chain FW3:

(SEQ ID NO: 35)

GVSSRFKGSGSGTEFTLTISGVQCDDAATYYC
1-3-1 Kappa Chain CDR3:

(SEQ ID NO: 36)

QQGVRVVDIDNS
1-3-1 Kappa Chain FW4:

(SEQ ID NO: 37)

FGGGTELVVK
1-25-2 Heavy Chain Variable Region
NUCLEOTIDE SEQUENCE:

(SEQ ID NO: 38)

GCCCAGTCGTTGGAGGAGTCCGGGGGAGACCTGGTCAAGCCTGAGGGA
TCCCTGACACTCACCTGCACAGCCTCTGGATTCTCCTTCAGTAGCAGC
TACTGGGGATGCTGGGTCCGCCAGGCTCCAGGGAAGGGGCCTGAGTGG
ATCGCATGCATTTATGGTGGTAGTAGTGGTACCACTTATTACCCGAAC
TGGGCGAAAGGCCGATTCTCCATCTCCAAAACCTCGTCGACCACGGTG
ACTCTGCAAATGGCCAGTCTGACAGCCGCGGACACGGCCACCTATTTC
TGTGCGAGAGTGACTAATGGTGGTGATTGGGATTTTAAATTGTGGGGC
CCAGGCACCCTGGTCACCATCTCTTCA
1-25-2 Heavy Chain FW1:

(SEQ ID NO: 39)

GCCCAGTCGTTGGAGGAGTCCGGGGGAGACCTGGTCAAGCCTGAGGGA
TCCCTGACACTCACCTGCACAGCCTCT
1-25-2 Heavy Chain CDR1:

(SEQ ID NO: 40)

GGATTCTCCTTCAGTAGCAGCTACTGGGGATGC
1-25-2 Heavy Chain FW2:

(SEQ ID NO: 41)

TGGGTCCGCCAGGCTCCAGGGAAGGGGCCTGAGTGGATCGCA
1-25-2 Heavy Chain CDR2:

(SEQ ID NO: 42)

TGCATTTATGGTGGTAGTAGTGGTACCACTTATTACCCGAACTGGGCG
AAAGGC
1-25-2 Heavy Chain FW3:

(SEQ ID NO: 43)

CGATTCTCCATCTCCAAAACCTCGTCGACCACG
1-25-2 Heavy Chain CDR3:

(SEQ ID NO: 44)

GTGACTCTGCAAATGGCCAGTCTGACAGCCGCGGACACGGCCACCTAT
TTCTGTGCGAGAGTGACTAATGGTGGTGATTGGGATTTTAAATTG
1-25-2 Heavy Chain FW4:

(SEQ ID NO: 45)

TGGGGCCCAGGCACCCTGGTCACCATCTCTTCA
PROTEIN SEQUENCE

(SEQ ID NO: 46)

AQSLEESGGDLVKPEGSLTLTCTASGFSFSSSYWGCWVRQAPGKGPEW
IACIYGGSSGTTYYPNWAKGRFSISKTSSTTVTLQMASLTAADTATYF
CARVTNGGDWDFKLWGPGTLVTISS
1-25-2 Heavy Chain FW1:

(SEQ ID NO: 47)

AQSLEESGGDLVKPEGSLTLTCTAS
1-25-2 Heavy Chain CDR1:

(SEQ ID NO: 48)

GFSFSSSYWGC
1-25-2 Heavy Chain FW2:

(SEQ ID NO: 49)

WVRQAPGKGPEWIA
1-25-2 Heavy Chain CDR2:

(SEQ ID NO: 50)

CIYGGSSGTTYYPNWAKG
1-25-2 Heavy Chain FW3:

(SEQ ID NO: 51)

RFSISKTSSTT
1-25-2 Heavy Chain CDR3:

(SEQ ID NO: 52)

VTLQMASLTAADTATYFCARVTNGGDWDFKL
1-25-2 Heavy Chain FW4:

(SEQ ID NO: 53)

WGPGTLVTISS
1-25-2 Kappa Chain Variable Region
NUCLEOTIDE SEQUENCE:

(SEQ ID NO: 54)

GAGCTCGTGCTGACCCAGACTCCAGCCTCTGTGGAGGTAGCTGTGGGA
GGCACAGTCACCATCAAGTGCCAGGCCAGTCAGACCATTAATAACTAC
TTGACCTGGTATCAGCAGAAACCAGGACAGCCTCCCAAGCTCCTGATC
TACAGGGCATCCACTCTGGCATCTGGGGTCCCATCGCGGTTCAAAGGC
AGTGGATCTGGGACAGAGTTCACTCTCACCATCAGCGACCTGGAGTGT
GCCGATGCTGCCACTTACTACTGTCAACAGACTGTTCGTGTGGTTGAT
GTTGATAATAGTTTCGGCGGAGGGACCGAGGTGGTGGTCAAA
1-25-2 Kappa Chain FW1:

(SEQ ID NO: 55)

GAGCTCGTGCTGACCCAGACTCCAGCCTCTGTGGAGGTAGCTGTGGGA
GGCACAGTCACCATCAAGTGC
1-25-2 Kappa Chain CDR1:

(SEQ ID NO: 56)

CAGGCCAGTCAGACCATTAATAACTACTTGACC
1-25-2 Kappa Chain FW2:

(SEQ ID NO: 57)

TGGTATCAGCAGAAACCAGGACAGCCTCCCAAGCTCCTGATCTAC
1-25-2 Kappa Chain CDR2:

(SEQ ID NO: 58)

AGGGCATCCACTCTGGCATCT
1-25-2 Kappa Chain FW3:

(SEQ ID NO: 59)

GGGGTCCCATCGCGGTTCAAAGGCAGTGGATCTGGGACAGAGTTCACT
CTCACCATCAGCGACCTGGAGTGTGCCGATGCTGCCACTTACTACTGT
1-25-2 Kappa Chain CDR3:

(SEQ ID NO: 60)

CAACAGACTGTTCGTGTGGTTGATGTTGATAATAGT
1-25-2 Kappa Chain FW4:

(SEQ ID NO: 61)

TTCGGCGGAGGGACCGAGGTGGTGGTCAAA
PROTEIN SEQUENCE

(SEQ ID NO: 62)

ELVLTQTPASVEVAVGGTVTIKCQASQTINNYLTWYQQKPGQPPKLLI
YRASTLASGVPSRFKGSGSGTEFTLTISDLECADAATYYCQQTVRVVD
VDNSFGGGTEVVVK
1-25-2 Kappa Chain FW1:

(SEQ ID NO: 63)

ELVLTQTPASVEVAVGGTVTIKC
1-25-2 Kappa Chain CDR1:

(SEQ ID NO: 64)

QASQTINNYLT
1-25-2 Kappa Chain FW2:

(SEQ ID NO: 65)

WYQQKPGQPPKLLIY
1-25-2 Kappa Chain CDR2:

(SEQ ID NO: 66)

RASTLAS
1-25-2 Kappa Chain FW3:

(SEQ ID NO: 67)

GVPSRFKGSGSGTEFTLTISDLECADAATYYC
1-25-2 Kappa Chain CDR3:

(SEQ ID NO: 68)

QQTVRVVDVDNS
1-25-2 Kappa Chain FW4:

(SEQ ID NO: 69)

FGGGTEVVVK
1-55-2 Heavy Chain Variable Region
NUCLEOTIDE SEQUENCE:

(SEQ ID NO: 70)

CAGCAGCAGCTGATGGAGTCCGGGGGAGACCTGGTCAAGCCTGAGGGA
TCCCTGACACTCACCTGCACAGCCTCTGGATTCTCCTTCAGTAGCAGC
TACTGGGGATGCTGGGTCCGCCAGGCTCCAGGGAAGGGGCCTGAGTGG
ATCGCATGCATTTATGGTGGTAGTAGTGGTACCACTTATTACCCGAAC
TGGGCGAAAGGCCGATTCTCCATCTCCAAAACCTCGTCGACCACGGTG
ACTCTGCAAATGGCCAGTCTGACAGCCGCGGACACGGCCACCTATTTC
TGTGCGAGAGTGACTAATGGTGGTGATTGGGATTTTAAATTGTGGGGC
CCAGGCACCCTGGTCACCGTCTCCTCA
1-55-2 Heavy Chain FW1:

(SEQ ID NO: 71)

CAGCAGCAGCTGATGGAGTCCGGGGGAGACCTGGTCAAGCCTGAGGGA
TCCCTGACACTCACCTGCACAGCCTCT
1-55-2 Heavy Chain CDR1:

(SEQ ID NO: 72)

GGATTCTCCTTCAGTAGCAGCTACTGGGGATGC
1-55-2 Heavy Chain FW2:

(SEQ ID NO: 73)

TGGGTCCGCCAGGCTCCAGGGAAGGGGCCTGAGTGGATCGCA
1-55-2 Heavy Chain CDR2:

(SEQ ID NO: 74)

TGCATTTATGGTGGTAGTAGTGGTACCACTTATTACCCGAACTGGGCG
AAAGGC
1-55-2 Heavy Chain FW3:

(SEQ ID NO: 75)

CGATTCTCCATCTCCAAAACCTCGTCGACCACG
1-55-2 Heavy Chain CDR3:

(SEQ ID NO: 76)

GTGACTCTGCAAATGGCCAGTCTGACAGCCGCGGACACGGCCACCTAT
TTCTGTGCGAGAGTGACTAATGGTGGTGATTGGGATTTTAAATTG
1-55-2 Heavy Chain FW4:

(SEQ ID NO: 77)

TGGGGCCCAGGCACCCTGGTCACCGTCTCCTCA
PROTEIN SEQUENCE

(SEQ ID NO: 78)

QQQLMESGGDLVKPEGSLTLTCTASGFSFSSSYWGCWVRQAPGKGPEW
IACIYGGSSGTTYYPNWAKGRFSISKTSSTTVTLQMASLTAADTATYF
CARVTNGGDWDFKLWGPGTLVTVSS
1-55-2 Heavy Chain FW1:

(SEQ ID NO: 79)

QQQLMESGGDLVKPEGSLTLTCTAS
1-55-2 Heavy Chain CDR1:

(SEQ ID NO: 80)

GFSFSSSYWGC
1-55-2 Heavy Chain FW2:

(SEQ ID NO: 81)

WVRQAPGKGPEWIA
1-55-2 Heavy Chain CDR2:

(SEQ ID NO: 82)

CIYGGSSGTTYYPNWAKG
1-55-2 Heavy Chain FW3:

(SEQ ID NO: 83)

RFSISKTSSTT
1-55-2 Heavy Chain CDR3:

(SEQ ID NO: 84)

VTLQMASLTAADTATYFCARVTNGGDWDFKL
1-55-2 Heavy Chain FW4:

(SEQ ID NO: 85)

WGPGTLVTVSS
1-55-2 Kappa Chain Variable Region
NUCLEOTIDE SEQUENCE:

(SEQ ID NO: 86)

GAGCTCGTGATGACCCAGACTCCAGCCTCTGTGGAGGTAGCTGTGGGA
GGCACAGTCACCATCAAGTGCCAGGCCAGTCAGAGCATTAATACCTAC
TTAGCCTGGTATCAGCAGAAACCAGGGCAGCCTCCCAAGCTCCTGATC
TACAGGGCATCCACTCTGGCATCTGGGGTCCCATCGCGGTTCAAAGGC
AGTGGATCTGGGACAGAGTTCACTCTCACCATTAGCGACCTGGAGTGT
GCCGATGCTGCCACTTACTACTGTCAACAGAGTGTTCGTGTTATTGAT
GTTGATAATACTTTCGGCGGAGGGACCGAGGTGGTCGTCAAAA
1-55-2 Kappa Chain FW1:

(SEQ ID NO: 87)

GAGCTCGTGATGACCCAGACTCCAGCCTC TGTGGAGGTAGCTGTGG
GAGGCACAGTCACCATCAAGTGC
1-55-2 Kappa Chain CDR1:

(SEQ ID NO: 88)

CAGGCCAGTCAGAGCATTAATACCTACTTAGCC
1-55-2 Kappa Chain FW2:

(SEQ ID NO: 89)

TGGTATCAGCAGAAACCAGGGCAGCCTCCCAAGCTCCTGATCTAC
1-55-2 Kappa Chain CDR2:

(SEQ ID NO: 90)

AGGGCATCCACTCTGGCATCT
1-55-2 Kappa Chain FW3:

(SEQ ID NO: 91)

GGGGTCCCATCGCGGTTCAAAGGCAGTGGATCTGGGACAGAGTTCACT
CTCACCATTAGCGACCTGGAGTGTGCCGATGCTGCCACTTACTACTGT
1-55-2 Kappa Chain CDR3:

(SEQ ID NO: 92)

CAACAGAGTGTTCGTGTTATTGATGTTGATAATACT
1-55-2 Kappa Chain FW4:

(SEQ ID NO: 93)

TTCGGCGGAGGGACCGAGGTGGTCGTCAAAA
PROTEIN SEQUENCE

(SEQ ID NO: 94)

ELVMTQTPASVEVAVGGTVTIKCQASQSINTYLAWYQQKPGQPPKLLI
YRASTLASGVPSRFKGSGSGTEFTLTISDLECADAATYYCQQSVRVID
VDNTFGGGTEVVVK
1-55-2 Kappa Chain FW1:

(SEQ ID NO: 95)

ELVMTQTPASVEVAVGGTVTIKC
1-55-2 Kappa Chain CDR1:

(SEQ ID NO: 96)

QASQSINTYLA
1-55-2 Kappa Chain FW2:

(SEQ ID NO: 97)

WYQQKPGQPPKLLIY
1-55-2 Kappa Chain CDR2:

(SEQ ID NO: 98)

RASTLAS
1-55-2 Kappa Chain FW3:

(SEQ ID NO: 99)

GVPSRFKGSGSGTEFTLTISDLECADAATYYC
1-55-2 Kappa Chain CDR3:

(SEQ ID NO: 100)

QQSVRVIDVDNT
1-55-2 Kappa Chain FW4:

(SEQ ID NO: 101)

FGGGTEVWK
Clone 8 Heavy Chain Variable Region
NUCLEOTIDE SEQUENCE:

(SEQ ID NO: 102)

CAACCAGCCATGGCCCAGTCGGTGGAGGAGTCCGGGGGAGGCCTGGTC
AAGCCTGAGGGATCCCTGACACTCTCCTGCAAAGCCTCTGGATTCTCC
TTCAGTAACAACTCCTACATATGCTGGGTCCGCCAGGCTCCAGGGAAG
GGTCTGGAGTGGATCGCATGCATTGAGTTTGGTCGTGGTAACTCACAC
TACGCGAGCTGGGCGAAAGGCCGATTCACCATCACCAGAAGCACCAGC
CTAAACACGGTGACTCTGCAACTGAACAGTCTGACAGCCGCGGACACG
GCCACCTATTTCTGTGCGAGAGGATCTCGTGGTGTTGGTTATGGTTAT
GGCTTGTGGGGCCCAGGCACCCTGGTCACCATCTCTTCAAAG
Clone 8 Heavy Chain FW1:

(SEQ ID NO: 103)

CAACCAGCCATGGCCCAGTCGGTGGAGGAGTCCGGGGGAGGCCTGGTC
AAGCCTGAGGGATCCCTGACACTCTCCTGCAAAGCCTCT
Clone 8 Heavy Chain CDR1:

(SEQ ID NO: 104)

GGATTCTCCTTCAGTAACAACTCCTACATATGC
Clone 8 Heavy Chain FW2:

(SEQ ID NO: 105)

TGGGTCCGCCAGGCTCCAGGGAAGGGTCTGGAGTGGATCGCA
Clone 8 Heavy Chain CDR2:

(SEQ ID NO: 106)

TGCATTGAGTTTGGTCGTGGTAACTCACACTACGCGAGCTGGGCGAAA
GGC
Clone 8 Heavy Chain FW3:

(SEQ ID NO: 107)

CGATTCACCATCACCAGAAGCACCAGCCTAAACACGGTGACTCTGCAA
CTGAACAGTCTGACAGCCGCGGACACGGCCACCTATTTCTGTGCGAGA
Clone 8 Heavy Chain CDR3:

(SEQ ID NO: 108)

GGATCTCGTGGTGTTGGTTATGGTTATGGCTTG
Clone 8 Heavy Chain FW4:

(SEQ ID NO: 109)

TGGGGCCCAGGCACCCTGGTCACCATCTCTTCAAAG
PROTEIN SEQUENCE

(SEQ ID NO: 110)

AQSVEESGGGLVKPEGSLTLSCKASGFSFSNNSYICWVRQAPGKGLEW
IACIEFGRGNSHYASWAKGRFTITRSTSLNTVTLQLNSLTAADTATYF
CARGSRGVGYGYGLWGPGTLVTISS
Clone 8 Heavy Chain FW1:

(SEQ ID NO: 111)

AQSVEESGGGLVKPEGSLTLSCKAS
Clone 8 Heavy Chain CDR1:

(SEQ ID NO: 112)

GFSFSNNSYIC
Clone 8 Heavy Chain FW2:

(SEQ ID NO: 113)

WVRQAPGKGLEWIA
Clone 8 Heavy Chain CDR2:

(SEQ ID NO: 114)

CIEFGRGNSHYASWAKG
Clone 8 Heavy Chain FW3:

(SEQ ID NO: 115)

RFTITRSTSLNTVTLQLNSLTAADTATYFCAR
Clone 8 Heavy Chain CDR3:

(SEQ ID NO: 116)

GSRGVGYGYGL
Clone 8 Heavy Chain FW4:

(SEQ ID NO: 117)

WGPGTLVTISS
Clone 8 Kappa Chain Variable Region
NUCLEOTIDE SEQUENCE:

(SEQ ID NO: 118)

GAGCTCGATATGACCCAGACTCCATCCCCTGTGTCTGCAGCTGTGGGA
GGCACAGTCACCATCAACTGCCAGGCCAGTCAGAGTGTTTACAGTAAC
AGCCGCTTATCCTGGTATCAGCAGAAACCAGGGCAGCCTCCCAAGCAA
CTGATCTATTCTGCATCCACTCTGGCATCTGGGGTCCCATCGCGGTTC
AAGGGCAGTGGATCTGGGACACATTTCACTCTCACCCTCAGCGGCGTA
CAGTGTGACGATGCTGCCACTTACTACTGTCAAGGCGAATTTGCTTGT
AGTAGTGCTGATTGTAGTGCTTTCGGCGGAGGGACCGAGCTGGAGATC
CTAAAG
Clone 8 Kappa Chain FW1:

(SEQ ID NO: 119)

GAGCTCGATATGACCCAGACTCCATCCCCTGTGTCTGCAGCTGTGGGA
GGCACAGTCACCATCAACTGC
Clone 8 Kappa Chain CDR1:

(SEQ ID NO: 120)

CAGGCCAGTCAGAGTGTTTACAGTAACAGCCGCTTATCC
Clone 8 Kappa Chain FW2:

(SEQ ID NO: 121)

TGGTATCAGCAGAAACCAGGGCAGCCTCCCAAGCAACTGATCTAT
Clone 8 Kappa Chain CDR2:

(SEQ ID NO: 122)

TCTGCATCCACTCTGGCATCT
Clone 8 Kappa Chain FW3:

(SEQ ID NO: 123)

GGGGTCCCATCGCGGTTCAAGGGCAGTGGATCTGGGACACATTTCACT
CTCACCCTCAGCGGCGTACAGTGTGACGATGCTGCCACTTACTACTGT
Clone 8 Kappa Chain CDR3:

(SEQ ID NO: 124)

CAAGGCGAATTTGCTTGTAGTAGTGCTGATTGTAGTGCT
Clone 8 Kappa Chain FW4:

(SEQ ID NO: 125)

TTCGGCGGAGGGACCGAGCTGGAGATCCTAAAG
PROTEIN SEQUENCE

(SEQ ID NO: 126)

ELDMTQTPSPVSAAVGGTVTINCQASQSVYSNSRLSWYQQKPGQPPKQ
LIYSASTLASGVPSRFKGSGSGTHFTLTLSGVQCDDAATYYCQGEFAC
SSADCSAFGGGTELEILK
Clone 8 Kappa Chain FW1:

(SEQ ID NO: 127)

ELDMTQTPSPVSAAVGGTVTINC
Clone 8 Kappa Chain CDR1:

(SEQ ID NO: 128)

QASQSVYSNSRLS
Clone 8 Kappa Chain FW2:

(SEQ ID NO: 129)

WYQQKPGQPPKQLIY
Clone 8 Kappa Chain CDR2:

(SEQ ID NO: 130)

SASTLAS
Clone 8 Kappa Chain FW3:

(SEQ ID NO: 131)

GVPSRFKGSGSGTHFTLTLSGVQCDDAATYYC
Clone 8 Kappa Chain CDR3:

(SEQ ID NO: 132)

QGEFACSSADCSA
Clone 8 Kappa Chain FW4:

(SEQ ID NO: 133)

FGGGTELEILK
PRIMERS USED FOR PCR AND SEQUENCING:

(SEQ ID NO: 134)

CCCAGTCACGACGTTGTAAAACG

(SEQ ID NO: 135)

AGCGGATAACAATTTCACACAGG

(SEQ ID NO: 136)

GCTGCCCAACCAGCCATGGCCCAGTCGGTGGAGGAGTCCRGG

(SEQ ID NO: 137)

GCTGCCCAACCAGCCATGGCCCAGTCGGTGAAGGAGTCCGAG

(SEQ ID NO: 138)

GCTGCCCAACCAGCCATGGCCCAGTCGYTGGAGGAGTCCGGG

(SEQ ID NO: 139)

GCTGCCCAACCAGCCATGGCCCAGSAGCAGCTGRTGGAGTCCGG

(SEQ ID NO: 140)

TGARGAGAYGGTGACCAGGGTGCC

(SEQ ID NO: 141)

GGGCCCAGGCGGCCGAGCTCGTGMTGACCCAGACTCCA

(SEQ ID NO: 142)

GGGCCCAGGCGGCCGAGCTCGATMTGACCCAGACTCCA

(SEQ ID NO: 143)

GGGCCCAGGCGGCCGAGCTCGTGATGACCCAGACTGAA

(SEQ ID NO: 144)

TAGGATCTCCAGCTCGGTCCC

(SEQ ID NO: 145)

TTTTGATTTCCACATTGGTGCC

(SEQ ID NO: 146)

TTTTGACSACCACCTCGGTCCC
1-3-1 Humanized Heavy Chain
NUCLEOTIDE SEQUENCE:

(SEQ ID NO: 147)

GAGGTGCAGCTGGTGGAGTCTGGCGGAGGCCTGGTGCAGCCAGGCGGA
AGCCTGAGGCTGTCCTGCGCCGCCTCCTCCGCCTCTGGCTTCACAATC
AACCGGAACTACTGGGTGCGGCAGGCCCCTGGCAAGGGCCTGGAGTGG
GTGAGCAGCATCTACGGCGGCTCCTCTGGAACCACATACTACACCAAC
TGGGCTAAGGGCCGCTTCACCATCTCTAGGGACAACTCTAAGAACACC
CTGTACCTGCAGATGAACAGCCTGAGGGCCGAGGACACCGCCGTGTAT
TACTGCGCCAGGGTGGACAACGGAGGCGATTGGTACTTCAGGCTGTGG
GGCCAGGGCACCCCCGTGACAGTGTCTTCCGCTTCTACCAAGGGCCCT
TCCGTGTTCCCTCTGGCCCCTTCCTCCAAGTCCACCTCCGGCGGCACC
GCCGCTCTGGGCTGCCTGGTGAAGGACTACTTCCCTGAGCCCGTGACC
GTGAGCTGGAACTCTGGCGCCCTGACCAGCGGAGTGCACACCTTCCCT
GCCGTGCTGCAGTCCTCCGGCCTGTACTCCCTGTCCTCCGTGGTGACC
GTGCCTTCCTCCTCCCTGGGCACCCAGACCTACATCTGCAACGTGAAC
CACAAGCCTTCCAACACCAAGGTGGACAAGAAGGTGGAGCCTAAGTCC
TGCGACAAGACCCACACCTGCCCTCCCTGCCCTGCCCCTGAGCTGCTG
GGCGGACCCTCCGTGTTCCTGTTCCCTCCTAAGCCTAAGGACACCCTG
ATGATCTCCCGGACCCCTGAGGTGACCTGCGTGGTGGTGGACGTGTCC
CACGAGGATCCTGAGGTGAAGTTCAATTGGTACGTGGACGGCGTGGAG
TGGCACAACGCCAAGACCAAGCCTCGGGAGGAACAGTACAACTCCACC
TACCGGGTGGTGTCTGTGCTGACCGTGCTGCACCAGGACTGGCTGAAC
GGCAAGGAATACAAGTGCAAGGTCTCCAACAAGGCCCTGCCTGCCCCC
ATCGAAAAGACCATCTCCAAGGCCAAGGGCCAGCCTCGCGAGCCTCAG
GTGTACACCCTGCCCCCCTCCCGGGACGAGCTGACCAAGAACCAGGTG
TCCCTGACCTGTCTGGTGAAGGGCTTCTACCCTTCCGATATCGCCGTG
GAGTGGGAGTCCAACGGCCAGCCTGAGAACAACTACAAGACCACCCCT
CCTGTGCTGGACTCCGACGGCTCCTTCTTCCTGTACTCCAAGCTGACC
GTGGACAAGTCCCGGTGGCAGCAGGGCAACGTGTTCTCCTGCTCCGTG
ATGCACGAGGCCCTGCACAACCACTACACCCAGAAGTCCCTGTCCCTG
TCTCCTGGCAAGTGA
HUMANIZED 1-3-1 HEAVY CHAIN VARIABLE REGION:

(SEQ ID NO: 148)

GAGGTGCAGCTGGTGGAGTCTGGCGGAGGCCTGGTGCAGCCAGGCGGA
AGCCTGAGGCTGTCCTGCGCCGCCTCCTCCGCCTCTGGCTTCACAATC
AACCGGAACTACTGGGTGCGGCAGGCCCCTGGCAAGGGCCTGGAGTGG
GTGAGCAGCATCTACGGCGGCTCCTCTGGAACCACATACTACACCAAC
TGGGCTAAGGGCCGCTTCACCATCTCTAGGGACAACTCTAAGAACACC
CTGTACCTGCAGATGAACAGCCTGAGGGCCGAGGACACCGCCGTGTAT
TACTGCGCCAGGGTGGACAACGGAGGCGATTGGTACTTCAGGCTGTGG
GGCCAGGGCACCCCCGTGACAGTGTCTTCC
HUMANIZED 1-3-1 HEAVY CHAIN FW1:

(SEQ ID NO: 149)

GAGGTGCAGCTGGTGGAGTCTGGCGGAGGCCTGGTGCAGCCAGGCGGA
AGCCTGAGGCTGTCCTGCGCCGCCTCC
HUMANIZED 1-3-1 HEAVY CHAIN CDR1:

(SEQ ID NO: 150)

TCCGCCTCTGGCTTCACAATCAACCGGAACTAC
HUMANIZED 1-3-1 HEAVY CHAIN FW2:

(SEQ ID NO: 151)

TGGGTGCGGCAGGCCCCTGGCAAGGGCCTGGAGTGGGTGAGC
HUMANIZED 1-3-1 HEAVY CHAIN CDR2:

(SEQ ID NO: 152)

AGCATCTACGGCGGCTCCTCTGGAACCACATACTACACCAACTGGGCT
AAGGGC
HUMANIZED 1-3-1 HEAVY CHAIN FW3:

(SEQ ID NO: 153)

CGCTTCACCATCTCTAGGGACAACTCTAAGAACACCCTGTACCTGCAG
ATGAACAGCCTGAGGGCCGAGGACACCGCCGTGTATTACTGCGCCAGG
HUMANIZED 1-3-1 HEAVY CHAIN CDR3:

(SEQ ID NO: 154)

GTGGACAACGGAGGCGATTGGTACTTCAGGCTG
HUMANIZED 1-3-1 HEAVY CHAIN FW4:

(SEQ ID NO: 155)

TGGGGCCAGGGCACCCCCGTGACAGTGTCTTCC
PROTEIN SEQUENCE:

(SEQ ID NO: 156)

EVQLVESGGGLVQPGGSLRLSCAASSASGFTINRNYWVRQAPGKGLEW
VSSIYGGSSGTTYYTNWAKGRFTISRDNSKNTLYLQMNSLRAEDTAVY
YCARVDNGGDINYFRLWGQGTPVTVSSASTKGPSVFPLAPSSKSTSGG
TAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCS
VMHEALHNHYTQKSLSLSPGK
HUMANIZED 1-3-1 HEAVY CHAIN VARIABLE REGION:

(SEQ ID NO: 157)

EVQLVESGGGLVQPGGSLRLSCAASSASGFTINRNYWVRQAPGKGLEW
VSSIYGGSSGTTYYTNWAKGRFTISRDNSKNTLYLQMNSLRAEDTAVY
YCARVDNGGDWYFRLWGQGTPVTVSS
HUMANIZED 1-3-1 HEAVY CHAIN FW1:

(SEQ ID NO: 158)

EVQLVESGGGLVQPGGSLRLSCAAS
HUMANIZED 1-3-1 HEAVY CHAIN CDR1:

(SEQ ID NO: 159)

SASGFTINRNY
HUMANIZED 1-3-1 HEAVY CHAIN FW2:

(SEQ ID NO: 160)

WVRQAPGKGLEWVS
HUMANIZED 1-3-1 HEAVY CHAIN CDR2:

(SEQ ID NO: 161)

SIYGGSSGTTYYTNWAKG
HUMANIZED 1-3-1 HEAVY CHAIN FW3:

(SEQ ID NO: 162)

RFTISRDNSKNTLYLQMNSLRAEDTAVYYCAR
HUMANIZED 1-3-1 HEAVY CHAIN CDR3:

(SEQ ID NO: 163)

VDNGGDWYFRL
HUMANIZED 1-3-1 HEAVY CHAIN FW4:

(SEQ ID NO: 164)

WGQGTPVTVSS
1-3-1 Humanized Kappa Light Chain
NUCLEOTIDE SEQUENCE:

(SEQ ID NO: 165)

GACATCCAGATGACACAGTCCCCCTCCACCCTGTCTGCCAGCGTGGGC
GATAGGGTGACCATCACATGTCAGGCCAGCCAGTCTATCTCTAGCTAC
CTGGCCTGGTACCAGCAGAAGCCTGGCAAGGCTCCCAAGCTGCTGATC
TATAGGGCTAGCACACTGGCCTCTGGAGTGCCTAGCCGGTTCTCTGGC
TCCGGCAGCGGAACCGAGTTTACCCTGACCATCTCCTCTCTGCAGCCT
GACGACTTCGCCACCTACTATTGCCAGCAGGGCGTGAGGGTGGTGGAC
ATCGACAACTCTTTCGGCCAGGGAACCAAGGTGGAGATCAAGAGGACC
GTGGCTGCCCCCAGCGTGTTCATCTTCCCCCCCTCTGACGAGCAGCTGA
AGTCCGGCACAGCTAGCGTGGTGTGCCTGCTGAACAATTTTTACCCCAG
AGAGGCTAAGGTGCAGTGGAAGGTGGACAATGCCCTGCAGAGCGGCAAC
AGCCAGGAGAGCGTGACCGAGCAGGACTCCAAGGACAGCACCTACTCCC
TGTCTTCCACCCTGACACTGAGCAAGGCCGACTACGAGAAGCACAAGGT
GTACGCTTGCGAGGTGACCCACCAGGGCCTGTCTTCTCCCGTGACCAAG
TCTTTCAACAGGGGCGAGTGCTGA
HUMANIZED 1-3-1 LIGHT CHAIN VARIABLE REGION:

(SEQ ID NO: 166)

GACATCCAGATGACACAGTCCCCCTCCACCCTGTCTGCCAGCGTGGGC
GATAGGGTGACCATCACATGTCAGGCCAGCCAGTCTATCTCTAGCTAC
CTGGCCTGGTACCAGCAGAAGCCTGGCAAGGCTCCCAAGCTGCTGATC
ATTAGGGCTAGCACACTGGCCTCTGGAGTGCCTAGCCGGTTCTCTGGC
TCCGGCAGCGGAACCGAGTTTACCCTGACCATCTCCTCTCTGCAGCCT
GACGACTTCGCCACCTACTATTGCCAGCAGGGCGTGAGGGTGGTGGAC
ATCGACAACTCTTTCGGCCAGGGAACCAAGGTGGAGATCAAG
HUMANIZED 1-3-1 LIGHT CHAIN FW1:

(SEQ ID NO: 167)

GACATCCAGATGACACAGTCCCCCTCCACCCTGTCTGCCAGCGTGGGC
GATAGGGTGACCATCACATGT
HUMANIZED 1-3-1 LIGHT CHAIN CDR1:

(SEQ ID NO: 168)

CAGGCCAGCCAGTCTATCTCTAGCTACCTGGCC
HUMANIZED 1-3-1 LIGHT CHAIN FW2:

(SEQ ID NO: 169)

TGGTACCAGCAGAAGCCTGGCAAGGCTCCCAAGCTGCTGATCTAT
HUMANIZED 1-3-1 LIGHT CHAIN CDR2:

(SEQ ID NO: 170)

AGGGCTAGCACACTGGCCTCT
HUMANIZED 1-3-1 LIGHT CHAIN FW3:

(SEQ ID NO: 171)

GGAGTGCCTAGCCGGTTCTCTGGCTCCGGCAGCGGAACCGAGTTTACC
CTGACCATCTCCTCTCTGCAGCCTGACGACTTCGCCACCTACTATTGC
HUMANIZED 1-3-1 LIGHT CHAIN CDR3:

(SEQ ID NO: 172)

CAGCAGGGCGTGAGGGTGGTGGACATCGACAACTCT
HUMANIZED 1-3-1 LIGHT CHAIN FW4:

(SEQ ID NO: 173)

CAGCAGGGCGTGAGGGTGGTGGACATCGACAACTCT
PROTEIN SEQUENCE:

(SEQ ID NO: 174)

DIQMTQSPSTLSASVGDRVTITCQASQSISSYLA WYQQKPGKAPKLLI
YRASTLASGVPSRFSGSGSGTEFTLTISSLQPDDFATYYCQQGVRWDI
DNSFGQGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
HUMANIZED 1-3-1 LIGHT CHAIN VARIABLE REGION:

(SEQ ID NO: 175)

DIQMTQSPSTLSASVGDRVTITCQASQSISSYLAWYQQKPGKAPKLLI
YRASTLASGVPSRFSGSGSGTEFTLTISSLQPDDFATYYCQQGVRWDI
DNSFGQGTKVEIKR
HUMANIZED 1-3-1 LIGHT CHAIN FW1:

(SEQ ID NO: 176)

DIQMTQSPSTLSASVGDRVTITC
HUMANIZED 1-3-1 LIGHT CHAIN CDR1:

(SEQ ID NO: 177)

QASQSISSYLA
HUMANIZED 1-3-1 LIGHT CHAIN FW2:

(SEQ ID NO: 178)

WYQQKPGKAPKLLIY
HUMANIZED 1-3-1 LIGHT CHAIN CDR2:

(SEQ ID NO: 179)

RASTLAS
HUMANIZED 1-3-1 LIGHT CHAIN FW3:

(SEQ ID NO: 180)

GVPSRFSGSGSGTEFTLTISSLQPDDFATYYC
HUMANIZED 1-3-1 LIGHT CHAIN CDR3:

(SEQ ID NO: 181)

QQGVRWDIDNS
HUMANIZED 1-3-1 LIGHT CHAIN FW4:

(SEQ ID NO: 182)

FGQGTKVEIKR
1-25-2 Humanized Heavy Chain
NUCLEOTIDE SEQUENCE:

(SEQ ID NO: 184)

ATGGGATGGAGCTGTATCATCCTCTTCTTGGTAGCAACAGCTACAGGT
GTCCACTCCGAGGTGCAGCTGGTGGAGTCTGGGGGAGGCTTGGTCCAG
CCTGGGGGGTCCCTGAGACTCTCCTGTGCAGCCTCTGGATTCTCCTTC
AGTAGCAGCTACTGGGGATGCCGCCAGGCTCCAGGGAAGGGGCTGGAG
TGGGTCTCATGCATTTATGGTGGTAGTAGTGGTACCACTTATTACCCG
AACTGGGCGAAAGGCAGATTCACCATCTCCAGAGACAATTCCAAGAAC
ACGCTGTATCTTCAAATGAACAGCCTGAGAGCCGAGGACACGGCTGTG
TATTACTGTGCGAGAGTGACTCTGCAAATGGCCAGTCTGACAGCCGCG
GACACGGCCACCTATTTCTGTGCGAGAGTGACTAATGGTGGTGATTGG
GATTTTAAATTGTGGGGCCAAGGGACCCCGGTCACCGTCTCCTCAGCC
TCCACCAAGGGCCCATCGGTCTTCCCCCTGGCACCCTCCTCCAAGAGC
ACCTCTGGGGGCACAGCGGCCCTGGGCTGCCTGGTCAAGGACTACTTC
CCCGAACCGGTGACGGTGTCGTGGAACTCAGGCGCCCTGACCAGCGGC
GTGCACACCTTCCCGGCTGTCCTACAGTCCTCAGGACTCTACTCCCTC
AGCAGCGTGGTGACCGTGCCCTCCAGCAGCTTGGGCACCCAGACCTAC
ATCTGCAACGTGAATCACAAGCCCAGCAACACCAAGGTGGACAAGAAA
GTTGAGCCCAAATCTTGTGACAAAACTCACACATGCCCACCGTGCCCA
GCACCTGAACTCCTGGGGGGACCGTCAGTCTTCCTCTTCCCCCCAAAA
CCCAAGGACACCCTCATGATCTCCCGGACCCCTGAGGTCACATGCGTG
GTGGTGGACGTGAGCCACGAAGACCCTGAGGTCAAGTTCAACTGGTAC
GTGGACGGCGTGGAGGTGCATAATGCCAAGACAAAGCCGCGGGAGGAG
CAGTACAACAGCACGTACCGTGTGGTCAGCGTCCTCACCGTCCTGCAC
CAGGACTGGCTGAATGGCAAGGAGTACAAGTGCAAGGTCTCCAACAAA
GCCCTCCCAGCCCCCATCGAGAAAACCATCTCCAAAGCCAAAGGGCAG
CCCCGAGAACCACAGGTGTACACCCTGCCCCCATCCCGGGATGAGCTG
ACCAAGAACCAGGTCAGCCTGACCTGCCTGGTCAAAGGCTTCTATCCC
AGCGACATCGCCGTGGAGTGGGAGAGCAATGGGCAGCCGGAGAACAAC
TACAAGACCACGCCTCCCGTGCTGGACTCCGACGGCTCCTTCTTCTTA
TATTCAAAGCTCACCGTGGACAAGAGCAGGTGGCAGCAGGGGAACGTC
TTCTCATGCTCCGTGATGCATGAGGCTCTGCACAACCACTACACGCAG
AAGAGCCTCTCCCTGTCTCCCGGGAAATGA
HUMANIZED 1-25-2 HEAVY CHAIN VARIABLE REGION:

(SEQ ID NO: 192)

GAGGTGCAGCTGGTGGAGTCTGGGGGAGGCTTGGTCCAGCCTGGGGGG
TCCCTGAGACTCTCCTGTGCAGCCTCTGGATTCTCCTTCAGTAGCAGC
TACTGGGGATGCCGCCAGGCTCCAGGGAAGGGGCTGGAGTGGGTCTCA
TGCATTTATGGTGGTAGTAGTGGTACCACTTATTACCCGAACTGGGCG
AAAGGCAGATTCACCATCTCCAGAGACAATTCCAAGAACACGCTGTAT
CTTCAAATGAACAGCCTGAGAGCCGAGGACACGGCTGTGTATTACTGT
GCGAGAGTGACTCTGCAAATGGCCAGTCTGACAGCCGCGGACACGGCC
ACCTATTTCTGTGCGAGAGTGACTAATGGTGGTGATTGGGATTTTAAA
TTGTGGGGCCAAGGGACCCCGGTCACCGTCTCCTCA
HUMANIZED 1-25-2 HEAVY CHAIN FW1:

(SEQ ID NO: 193)

GAGGTGCAGCTGGTGGAGTCTGGGGGAGGCTTGGTCCAGCCTGGGGGG
TCCCTGAGACTCTCCTGTGCAGCCTCT
HUMANIZED 1-25-2 HEAVY CHAIN CDR1:

(SEQ ID NO: 194)

GGATTCTCCTTCAGTAGCAGCTACTGGGGA
HUMANIZED 1-25-2 HEAVY CHAIN FW2:

(SEQ ID NO: 195)

TGCCGCCAGGCTCCAGGGAAGGGGCTGGAGTGGGTCTCA
HUMANIZED 1-25-2 HEAVY CHAIN CDR2:

(SEQ ID NO: 196)

TGCATTTATGGTGGTAGTAGTGGTACCACTTATTACCCGAACTGGGCG
AAAGGC
HUMANIZED 1-25-2 HEAVY CHAIN FW3:

(SEQ ID NO: 197)

AGATTCACCATCTCCAGAGACAATTCCAAGAACACGCTGTATCTTCAA
ATGAACAGCCTGAGAGCCGAGGACACGGCTGTGTATTACTGTGCGAGA
GTGACTCTGCAAATGGCCAGTCTGACAGCCGCGGACACGGCCACCTAT
TTCTGTGCGAGA
HUMANIZED 1-25-2 HEAVY CHAIN CDR3:

(SEQ ID NO: 198)

GTGACTAATGGTGGTGATTGGGATTTTAAATTG
HUMANIZED 1-25-2 HEAVY CHAIN FW4:

(SEQ ID NO: 199)

TGGGGCCAAGGGACCCCGGTCACCGTCTCCTCA
PROTEIN SEQUENCE:

(SEQ ID NO: 185)

MGWSCIILFLVATATGVHSEVQLVESGGGLVQPGGSLRLSCAASGFSF
SSSYWGCRQAPGKGLEWVSCIYGGSSGTTYYPNWAKGRFTISRDNSKN
TLYLQMNSLRAEDTAVYYCARVTLQMASLTAADTATYFCARVTNGGDW
DFKLWGQGTPVTVSSASTKGPSVFPLAPSSKSTSGGTAALGCLVKDYF
PEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENN
PYKTTPVLDSDGSFFLYSKLTVDKSRWQQGNVFSCSVMHEALHNHYTQ
KSLSLSPGK
HUMANIZED 1-25-2 HEAVY CHAIN VARIABLE REGION:

(SEQ ID NO: 200)

EVQLVESGGGLVQPGGSLRLSCAASGFSFSSSYWGCRQAPGKGLEWVS
CIYGGSSGTTYYPNWAKGRFTISRDNSKNTLYLQMNSLRAEDTAVYYC
ARVTLQMASLTAADTATYFCARVTNGGDWDFKLWGQGTPVTVSS
HUMANIZED 1-25-2 HEAVY CHAIN FW1:

(SEQ ID NO: 201)

EVQLVESGGGLVQPGGSLRLSCAAS
HUMANIZED 1-25-2 HEAVY CHAIN CDR1:

(SEQ ID NO: 202)

GFSFSSSYWG
HUMANIZED 1-25-2 HEAVY CHAIN FW2:

(SEQ ID NO: 203)

CRQAPGKGLEWVS
HUMANIZED 1-25-2 HEAVY CHAIN CDR2:

(SEQ ID NO: 204)

CIYGGSSGTTYYPNWAKG
HUMANIZED 1-25-2 HEAVY CHAIN FW3:

(SEQ ID NO: 205)

RFTISRDNSKNTLYLQMNSLRAEDTAVYYCARVTLQMASLTAADTATY
FCAR
HUMANIZED 1-25-2 HEAVY CHAIN CDR3:

(SEQ ID NO: 206)

VTNGGDWDFKL
HUMANIZED 1-25-2 HEAVY CHAIN FW4:

(SEQ ID NO: 207)

WGQGTPVTVSS
1-25-2 Humanized Kappa Light Chain
NUCLEOTIDE SEQUENCE:

(SEQ ID NO: 186)

ATGGGATGGAGCTGTATCATCCTCTTCTTGGTAGCAACAGCTACAGGT
GTCCACTCCGACATCCAGATGACCCAGTCTCCATCCTCCCTGTCTGCA
TCTGTAGGAGACAGAGTCACCATCACTTGCCAGGCCAGTCAGACCATT
AATAACTACTTGACCTGGTATCAGCAGAAACCAGGGAAAGTTCCTAAG
CTCCTGATCTATAGGGCATCCACTCTGGCATCTGGGGTCCCATCTCGG
TTCAGTGGCAGTGGATCTGGGACAGATTTCACTCTCACCATCAGCAGC
CTGCAGCCTGAAGATGTTGCAACTTATTACTGTCAACAGACTGTTCGT
GTGGTTGATGTTGATAATAGTTTCGGCCAAGGGACCAAGGTGGAAATC
AAACGAACTGTGGCTGCACCATCTGTCTTCATCTTCCCGCCATCTGAT
GAGCAGTTGAAATCTGGAACTGCCTCTGTTGTGTGCCTGCTGAATAAC
TTCTATCCCAGAGAGGCCAAAGTACAGTGGAAGGTGGATAACGCCCTC
CAATCGGGTAACTCCCAGGAGAGTGTCACAGAGCAGGACAGCAAGGAC
AGCACCTACAGCCTCAGCAGCACCCTGACGCTGAGCAAAGCAGACTAC
GAGAAACACAAAGTCTACGCCTGCGAAGTCACCCATCAGGGCCTGAGC
TCGCCCGTCACAAAGAGCTTCAACAGGGGAGAGTGTTAA
HUMANIZED 1-25-2 LIGHT CHAIN VARIABLE REGION:

(SEQ ID NO: 208)

GACATCCAGATGACCCAGTCTCCATCCTCCCTGTCTGCATCTGTAGGA
GACAGAGTCACCATCACTTGCCAGGCCAGTCAGACCATTAATAACTAC
TTGACCTGGTATCAGCAGAAACCAGGGAAAGTTCCTAAGCTCCTGATC
TATAGGGCATCCACTCTGGCATCTGGGGTCCCATCTCGGTTCAGTGGC
AGTGGATCTGGGACAGATTTCACTCTCACCATCAGCAGCCTGCAGCCT
GAAGATGTTGCAACTTATTACTGTCAACAGACTGTTCGTGTGGTTGAT
GTTGATAATAGTTTCGGCCAAGGGACCAAGGTGGAAATCAAA
HUMANIZED 1-25-2 LIGHT CHAIN FW1:

(SEQ ID NO: 209)

GACATCCAGATGACCCAGTCTCCATCCTCCCTGTCTGCATCTGTAGGA
GACAGAGTCACCATCACTTGC
HUMANIZED 1-25-2 LIGHT CHAIN CDR1:

(SEQ ID NO: 210)

CAGGCCAGTCAGACCATTAATAACTACTTGACC
HUMANIZED 1-25-2 LIGHT CHAIN FW2:

(SEQ ID NO: 211)

TGGTATCAGCAGAAACCAGGGAAAGTTCCTAAGCTCCTGATCTAT
HUMANIZED 1-25-2 LIGHT CHAIN CDR2:

(SEQ ID NO: 212)

AGGGCATCCACTCTGGCATCT
HUMANIZED 1-25-2 LIGHT CHAIN FW3:

(SEQ ID NO: 213)

GGGGTCCCATCTCGGTTCAGTGGCAGTGGATCTGGGACAGATTTCACT
CTCACCATCAGCAGCCTGCAGCCTGAAGATGTTGCAACTTATTACTGT
HUMANIZED 1-25-2 LIGHT CHAIN CDR3:

(SEQ ID NO: 214)

CAACAGACTGTTCGTGTGGTTGATGTTGATAATAGT
HUMANIZED 1-25-2 LIGHT CHAIN FW4:

(SEQ ID NO: 215)

TTCGGCCAAGGGACCAAGGTGGAAATCAAA
PROTEIN SEQUENCE:

(SEQ ID NO: 187)

MGWSCIILFLVATATGVHSDIQMTQSPSSLSASVGDRVTITCQASQTI
NNYLTWYQQKPGKVPKLLIYRASTLASGVPSRFSGSGSGTDFTLTISS
LQPEDVATYYCQQTVRVVDVDNSFGQGTKVEIKRTVAAPSVFIFPPSD
EQLKSGTASVVCLLNNFYPREAKVQWKVDNALQSGNSQESVTEQDSKD
STYSLSSTLTLSKADYEKHKVYACEVTHQGLSSPVTKSFNRGEC
HUMANIZED 1-25-2 LIGHT CHAIN VARIABLE REGION:

(SEQ ID NO: 216)

DIQMTQSPSSLSASVGDRVTITCQASQTINNYLTWYQQKPGKVPKLLI
YRASTLASGVPSRFSGSGSGTDFTLTISSLQPEDVATYYCQQTVRVVD
VDNSFGQGTKVEIK
HUMANIZED 1-25-2 LIGHT CHAIN FW1:

(SEQ ID NO: 217)

DIQMTQSPSSLSASVGDRVTITC
HUMANIZED 1-25-2 LIGHT CHAIN CDR1:

(SEQ ID NO: 218)

QASQTINNYLT
HUMANIZED 1-25-2 LIGHT CHAIN FW2:

(SEQ ID NO: 219)

WYQQKPGKVPKLLIY
HUMANIZED 1-25-2 LIGHT CHAIN CDR2:

(SEQ ID NO: 220)

RASTLAS
HUMANIZED 1-25-2 LIGHT CHAIN FW3:

(SEQ ID NO: 221)

GVPSRFSGSGSGTDFTLTISSLQPEDVATYYC
HUMANIZED 1-25-2 LIGHT CHAIN CDR3:

(SEQ ID NO: 222)

QQTVRVVDVDNS
HUMANIZED 1-25-2 LIGHT CHAIN FW4:

(SEQ ID NO: 223)

FGQGTKVEIK
1-55-2 Humanized Heavy Chain
NUCLEOTIDE SEQUENCE:

(SEQ ID NO: 188)

ATGGGATGGAGCTGTATCATCCTCTTCTTGGTAGCAACAGCTACAGGT
GTCCACTCCGAGGTGCAGCTGGTGGAGTCTGGGGGAGGCTTGGTCCAG
CCTGGGGGGTCCCTGAGACTCTCCTGTGCAGCCTCTGGATTCTCCTTC
AGTAGCAGCTACTGGGGATGCTGGGTCCGCCAGGCTCCAGGGAAGGGG
CTGGAGTGGGTCTCATGCATTTATGGTGGTAGTAGTGGTACCACTTAT
TACCCGAACTGGGCGAAAGGCAGATTCACCATCTCCAGAGACAATTCC
AAGAACACGCTGTATCTTCAAATGAACAGCCTGAGAGCCGAGGACACG
GCTGTGTATTACTGTGCGAGAGTGACTCTGCAAATGGCCAGTCTGACA
GCCGCGGACACGGCCACCTATTTCTGTGCGAGAGTGACTAATGGTGGT
GATTGGGATTTTAAATTGTGGGGCCAAGGGACCCCGGTCACCGTCTCC
TCAGCCTCCACCAAGGGCCCATCGGTCTTCCCCCTGGCACCCTCCTCC
AAGAGCACCTCTGGGGGCACAGCGGCCCTGGGCTGCCTGGTCAAGGAC
TACTTCCCCGAACCGGTGACGGTGTCGTGGAACTCAGGCGCCCTGACC
AGCGGCGTGCACACCTTCCCGGCTGTCCTACAGTCCTCAGGACTCTAC
TCCCTCAGCAGCGTGGTGACCGTGCCCTCCAGCAGCTTGGGCACCCAG
ACCTACATCTGCAACGTGAATCACAAGCCCAGCAACACCAAGGTGGAC
AAGAAAGTTGAGCCCAAATCTTGTGACAAAACTCACACATGCCCACCG
TGCCCAGCACCTGAACTCCTGGGGGGACCGTCAGTCTTCCTCTTCCCC
CCAAAACCCAAGGACACCCTCATGATCTCCCGGACCCCTGAGGTCACA
TGCGTGGTGGTGGACGTGAGCCACGAAGACCCTGAGGTCAAGTTCAAC
TGGTACGTGGACGGCGTGGAGGTGCATAATGCCAAGACAAAGCCGCGG
GAGGAGCAGTACAACAGCACGTACCGTGTGGTCAGCGTCCTCACCGTC
CTGCACCAGGACTGGCTGAATGGCAAGGAGTACAAGTGCAAGGTCTCC
AACAAAGCCCTCCCAGCCCCCATCGAGAAAACCATCTCCAAAGCCAAA
GGGCAGCCCCGAGAACCACAGGTGTACACCCTGCCCCCATCCCGGGAT
GAGCTGACCAAGAACCAGGTCAGCCTGACCTGCCTGGTCAAAGGCTTC
TATCCCAGCGACATCGCCGTGGAGTGGGAGAGCAATGGGCAGCCGGAG
AACAACTACAAGACCACGCCTCCCGTGCTGGACTCCGACGGCTCCTTC
TTCTTATATTCAAAGCTCACCGTGGACAAGAGCAGGTGGCAGCAGGGG
AACGTCTTCTCATGCTCCGTGATGCATGAGGCTCTGCACAACCACTAC
ACGCAGAAGAGCCTCTCCCTGTCTCCCGGGAAATGA
HUMANIZED 1-55-2 HEAVY CHAIN VARIABLE REGION:

(SEQ ID NO: 224)

GAGGTGCAGCTGGTGGAGTCTGGGGGAGGCTTGGTCCAGCCTGGGGGG
TCCCTGAGACTCTCCTGTGCAGCCTCTGGATTCTCCTTCAGTAGCAGC
TACTGGGGATGCTGGGTCCGCCAGGCTCCAGGGAAGGGGCTGGAGTGG
GTCTCATGCATTTATGGTGGTAGTAGTGGTACCACTTATTACCCGAAC
TGGGCGAAAGGCAGATTCACCATCTCCAGAGACAATTCCAAGAACACG
CTGTATCTTCAAATGAACAGCCTGAGAGCCGAGGACACGGCTGTGTAT
TACTGTGCGAGAGTGACTCTGCAAATGGCCAGTCTGACAGCCGCGGAC
ACGGCCACCTATTTCTGTGCGAGAGTGACTAATGGTGGTGATTGGGAT
TTTAAATTGTGGGGCCAAGGGACCCCGGTCACCGTCTCCTCA
HUMANIZED 1-55-2 HEAVY CHAIN FW1:

(SEQ ID NO: 225)

GAGGTGCAGCTGGTGGAGTCTGGGGGAGGCTTGGTCCAGCCTGGGGGG
TCCCTGAGACTCTCCTGTGCAGCCTCT
HUMANIZED 1-55-2 HEAVY CHAIN CDR1:

(SEQ ID NO: 226)

GGATTCTCCTTCAGTAGCAGCTACTGGGGATGC
HUMANIZED 1-55-2 HEAVY CHAIN FW2:

(SEQ ID NO: 227)

TGGGTCCGCCAGGCTCCAGGGAAGGGGCTGGAGTGGGTCTCA
HUMANIZED 1-55-2 HEAVY CHAIN CDR2:

(SEQ ID NO: 228)

TGCATTTATGGTGGTAGTAGTGGTACCACTTATTACCCGAACTGGGCG
AAAGGC
HUMANIZED 1-55-2 HEAVY CHAIN FW3:

(SEQ ID NO: 229)

AGATTCACCATCTCCAGAGACAATTCCAAGAACACGCTGTATCTTCAA
ATGAACAGCCTGAGAGCCGAGGACACGGCTGTGTATTACTGTGCGAGA
GTGACTCTGCAAATGGCCAGTCTGACAGCCGCGGACACGGCCACCTAT
TTCTGTGCGAGA
HUMANIZED 1-55-2 HEAVY CHAIN CDR3:

(SEQ ID NO: 230)

GTGACTAATGGTGGTGATTGGGATTTTAAATTG
HUMANIZED 1-55-2 HEAVY CHAIN FW4:

(SEQ ID NO: 231)

TGGGGCCAAGGGACCCCGGTCACCGTCTCCTCA
PROTEIN SEQUENCE:

(SEQ ID NO: 189)

MGWSCIILFLVATATGVHSEVQLVESGGGLVQPGGSLRLSCAASGFSF
SSSYWGCWVRQAPGKGLEWVSCIYGGSSGTTYYPNWAKGRFTISRDNS
KNTLYLQMNSLRAEDTAVYYCARVTLQMASLTAADTATYFCARVTNGG
DWDFKLWGQGTPVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
HUMANIZED 1-55-2 HEAVY CHAIN VARIABLE REGION:

(SEQ ID NO: 232)

EVQLVESGGGLVQPGGSLRLSCAASGFSFSSSYWGCWVRQAPGKGLEW
VSCIYGGSSGTTYYPNWAKGRFTISRDNSKNTLYLQMNSLRAEDTAVY
YCARVTLQMASLTAADTATYFCARVTNGGDWDFKLWGQGTPVTVSS
HUMANIZED 1-55-2 HEAVY CHAIN FW1:

(SEQ ID NO: 233)

EVQLVESGGGLVQPGGSLRLSCAAS
HUMANIZED 1-55-2 HEAVY CHAIN CDR1:

(SEQ ID NO: 234)

GFSFSSSYWGC
HUMANIZED 1-55-2 HEAVY CHAIN FW2:

(SEQ ID NO: 235)

WVRQAPGKGLEWVS
HUMANIZED 1-55-2 HEAVY CHAIN CDR2:

(SEQ ID NO: 236)

CIYGGSSGTTYYPNWAKG
HUMANIZED 1-55-2 HEAVY CHAIN FW3:

(SEQ ID NO: 237)

RFTISRDNSKNTLYLQMNSLRAEDTAVYYCARVTLQMASLTAADTATY
FCAR
HUMANIZED 1-55-2 HEAVY CHAIN CDR3:

(SEQ ID NO: 238)

VTNGGDWDFKL
HUMANIZED 1-55-2 HEAVY CHAIN FW4:

(SEQ ID NO: 239)

WGQGTPVTVSS
1-55-2 Humanized Kappa Light Chain
NUCLEOTIDE SEQUENCE:

(SEQ ID NO: 190)

ATGGGATGGAGCTGTATCATCCTCTTCTTGGTAGCAACAGCTACAGGT
GTCCACTCCGACATCCAGATGACCCAGTCTCCATCCTCCCTGTCTGCA
TCTGTAGGAGACAGAGTCACCATCACTTGCCAGGCCAGTCAGAGCATT
AATACCTACTTAGCCTGGTATCAGCAGAAACCAGGGAAAGTTCCTAAG
CTCCTGATCTATAGGGCATCCACTCTGGCATCTGGGGTCCCATCTCGG
TTCAGTGGCAGTGGATCTGGGACAGATTTCACTCTCACCATCAGCAGC
CTGCAGCCTGAAGATGTTGCAACTTATTACTGTCAACAGAGTGTTCGT
GTTATTGATGTTGATAATACTTTCGGCCAAGGGACCAAGGTGGAAATC
AAACGAACTGTGGCTGCACCATCTGTCTTCATCTTCCCGCCATCTGAT
GAGCAGTTGAAATCTGGAACTGCCTCTGTTGTGTGCCTGCTGAATAAC
TTCTATCCCAGAGAGGCCAAAGTACAGTGGAAGGTGGATAACGCCCTC
CAATCGGGTAACTCCCAGGAGAGTGTCACAGAGCAGGACAGCAAGGAC
AGCACCTACAGCCTCAGCAGCACCCTGACGCTGAGCAAAGCAGACTAC
GAGAAACACAAAGTCTACGCCTGCGAAGTCACCCATCAGGGCCTGAGC
TCGCCCGTCACAAAGAGCTTCAACAGGGGAGAGTGTTAA
HUMANIZED 1-55-2 LIGHT CHAIN VARIABLE REGION:

(SEQ ID NO: 240)

GACATCCAGATGACCCAGTCTCCATCCTCCCTGTCTGCATCTGTAGGA
GACAGAGTCACCATCACTTGCCAGGCCAGTCAGAGCATTAATACCTAC
TTAGCCTGGTATCAGCAGAAACCAGGGAAAGTTCCTAAGCTCCTGATC
TATAGGGCATCCACTCTGGCATCTGGGGTCCCATCTCGGTTCAGTGGC
AGTGGATCTGGGACAGATTTCACTCTCACCATCAGCAGCCTGCAGCCT
GAAGATGTTGCAACTTATTACTGTCAACAGAGTGTTCGTGTTATTGAT
GTTGATAATACTTTCGGCCAAGGGACCAAGGTGGAAATCAAA
HUMANIZED 1-55-2 LIGHT CHAIN FW1:

(SEQ ID NO: 241)

GACATCCAGATGACCCAGTCTCCATCCTCCCTGTCTGCATCTGTAGGA
GACAGAGTCACCATCACTTGC
HUMANIZED 1-55-2 LIGHT CHAIN CDR1:

(SEQ ID NO: 242)

CAGGCCAGTCAGAGCATTAATACCTACTTAGCC
HUMANIZED 1-55-2 LIGHT CHAIN FW2:

(SEQ ID NO: 243)

TGGTATCAGCAGAAACCAGGGAAAGTTCCTAAGCTCCTGATCTAT
HUMANIZED 1-55-2 LIGHT CHAIN CDR2:

(SEQ ID NO: 244)

AGGGCATCCACTCTGGCATCT
HUMANIZED 1-55-2 LIGHT CHAIN FW3:

(SEQ ID NO: 245)

GGGGTCCCATCTCGGTTCAGTGGCAGTGGATCTGGGACAGATTTCACT
CTCACCATCAGCAGCCTGCAGCCTGAAGATGTTGCAACTTATTACTGT
HUMANIZED 1-55-2 LIGHT CHAIN CDR3:

(SEQ ID NO: 246)

CAACAGAGTGTTCGTGTTATTGATGTTGATAATACT
HUMANIZED 1-55-2 LIGHT CHAIN FW4:

(SEQ ID NO: 247)

TTCGGCCAAGGGACCAAGGTGGAAATCAAA
PROTEIN SEQUENCE:

(SEQ ID NO: 191)

MGWSCIILFLVATATGVHSDIQMTQSPSSLSASVGDRVTITCQASQSI
NTYLAWYQQKPGKVPKLLIYRASTLASGVPSRFSGSGSGTDFTLTISS
LQPEDVATYYCQQSVRVIDVDNTFGQGTKVEIKRTVAAPSVFIFPPSD
EQLKSGTASVVCLLNNFYPREAKVQWKVDNALQSGNSQESVTEQDSKD
STYSLSSTLTLSKADYEKHKVYACEVTHQGLSSPVTKSFNRGEC
HUMANIZED 1-55-2 LIGHT CHAIN VARIABLE REGION:

(SEQ ID NO: 248)

DIQMTQSPSSLSASVGDRVTITCQASQSINTYLAWYQQKPGKVPKLLI
YRASTLASGVPSRFSGSGSGTDFTLTISSLQPEDVATYYCQQSVRVID
VDNTFGQGTKVEIK
HUMANIZED 1-55-2 LIGHT CHAIN FW1:

(SEQ ID NO: 249)

DIQMTQSPSSLSASVGDRVTITC
HUMANIZED 1-55-2 LIGHT CHAIN CDR1:

(SEQ ID NO: 250)

QASQSINTYLA
HUMANIZED 1-55-2 LIGHT CHAIN FW2:

(SEQ ID NO: 251)

WYQQKPGKVPKLLIY
HUMANIZED 1-55-2 LIGHT CHAIN CDR2:

(SEQ ID NO: 252)

RASTLAS
HUMANIZED 1-55-2 LIGHT CHAIN FW3:

(SEQ ID NO: 253)

GVPSRFSGSGSGTDFTLTISSLQPEDVATYYC
HUMANIZED 1-55-2 LIGHT CHAIN CDR3:

(SEQ ID NO: 254)

QQSVRVIDVDNT
HUMANIZED 1-55-2 LIGHT CHAIN FW4:

(SEQ ID NO: 255)

FGQGTKVEIK
HUMAN ENDOSIALIN-FC FUSION PROTEIN
Underlined sequence indicates extracellular
domain of hTEM1.
Bolded text is the in-frame HpaI site.
Italicized text is the enterkinase site.
UPPER CASE TEXT is the murine IgG2b Fc fragment.

(SEQ ID NO: 183)

atgctgctgcgcctgttgctggcctgggcggccgcagggcccac
actgggccaggacccctgggctgctgagccccgtgccgcctgcg
gccccagcagctgctacgctctcttcccacggcgccgcaccttc
ctggaggcctggcgggcctgccgcgagctggggggcgacctgg
ccactcctcggacccccgaggaggcccagcgtgtggacagcctg
gtgggtgcgggcccagccagccggctgctgtggatcgggctgca
gcggcaggcccggcaatgccagctgcagcgcccactgcgcggc
ttcacgtggaccacaggggaccaggacacggctttcaccaact
gggcccagccagcctctggaggcccctgcccggcccagcgctgt
gtggccctggaggcaagtggcgagcaccgctggctggagggct
cgtgcacgctggctgtcgacggctacctgtgccagtttggcttcg
agggcgcctgcccggcgctgcaagatgaggcgggccaggccgg
cccagccgtgtataccacgcccttccacctggtctccacagagt
ttgagtggctgcccttcggctctgtggccgctgtgcagtgccag
gctggcaggggagcctctctgctctgcgtgaagcagcctgaggg
aggtgtgggctggtcacgggctgggcccctgtgcctggggactg
gctgcagccctgacaacgggggctgcgaacacgaatgtgtgga
ggaggtggatggtcacgtgtcctgccgctgcactgagggcttccg
gctggcagcagacgggcgcagttgcgaggacccctgtgcccag
gctccgtgcgagcagcagtgtgagcccggtgggccacaaggcta
cagctgccactgtcgcctgggtttccggccagcggaggatgatc
cgcaccgctgtgtggacacagatgagtgccagattgccggtgtg
tgccagcagatgtgtgtcaactacgttggtggcttcgagtgtta
ttgtagcgagggacatgagctggaggctgatggcatcagctgca
gccctgcaggggccatgggtgcccaggcttcccaggacctcgga
gatgagttgctggatgacggggaggatgaggaagatgaagacg
aggcctggaaggccttcaacggtggctggacggagatgcctgg
gatcctgtggatggagcctacgcagccgcctgactttgccctggc
ctatagaccgagcttcccagaggacagagagccacagataccc
tacccggagcccacctggccacccccgctcagtgcccccagggt
cccctaccactcctcagtgctctccgtcacccggcctgtggtgg
tctctgccacgcatcccacactgccttctgcccaccagcctcctg
tgatccctgccacacacccagctttgtcccgtgaccaccagatc
cccgtgatcgcagccaactatccagatctgccttctgcctacca
acccggtattctctgtctctctcattcagcacagcctcctgccc
accagccccctatgatctcaaccaaatatccggagctcttccct
gcccaccagtcccccatgtttccagacacccgggtcgctggcac
ccagaccaccactcatttgcctggaatcccacctaaccatgccc
ctctggtcaccaccctcggtgcccagctaccccctcaagcccca
gatgcccttgtcctcagaacccaggccacccagcttcccattat
cccaactgcccagccctctctgaccaccacctccaggtcccctg
tgtctcctgcccatcaaatctctgtgcctgctgccacccagccc
gcagccctccccaccctcctgccctctcagagccccactaacca
gacctcacccatcagccctacacatccccattccaaagcccccc
aaatcccaagggaagatggccccagtcccaagttggccctgtgg
ctgccctcaccagctcccacagcagccccaacagccctgggg
gaggctggtcttgccgagcacagccagagggatgaccgg gttaac gac
gacgacgacaaaGAGCCCAGCGGACCAATTTCAACAATCAACCCCTCT
CCTCCATCCAAGGAGTCTCACAAAAGCCCAGCTCCTAACCTCGAGGGT
GGACCATCCGTCTTCATCTTCCCTCCAAATATCAAGGATGTACTCATG
ATCTCCCTGACACCCAAGGTCACGTGTGTGGTGGTGGATGTGAGCGAG
GATGACCCAGACGTCCAGATCAGCTGGTTTGTGAACAACGTGGAAGTA
CACACAGCYCAGACACAAACCCATAGAGAGGATTACAACAGTACTATC
CGGGTGGTCAGCACCCTCCCCATCCAGCACCAGGACTGGATGAGTGGC
AAGGAGTTCAAATGCAAGGTCAACAACAAAGACCTCCCATCACCCATC
GAGAGAACCATCTCAAAAATTAAAGGGCTAGTCAGAGCTCCACAAGTA
TACATCTTGCCGCCACCAGCAGAGCAGTTGTCCAGGAAAGATGTCAGT
CTCACTTGCCTGGTCGTGGGCTTCAACCCTGGAGACATCAGTGTGGAG
TGGACCAGCAATGGGCATACAGAGGAGAACTACAAGGACACCGCACCA
GTCCTGGACTCTGACGGTTCTTACTTCATATATAGCAAGCTCAATATG
AAAACAAGCAAGTGGGAGAAAACAGATTCCTTCTCATGCAACGTGAGA
CACGAGGGTCTGAAAAATTACTACCTGAAGAAGACCATCTCCCGGTCT
CCGGGTAAATGA
Key:
In the heavy and light chain sequences and the heavy and light chain variable region sequences, CDRs are indicated with bold text.

What is claimed is:

1. A rabbit monoclonal antibody or an antigen-binding portion thereof that specifically binds endosialin.

2. The rabbit monoclonal antibody or antigen-binding portion of claim 1, which is an endosialin antagonist.

3. The rabbit monoclonal antibody or antigen-binding portion of claim 1, wherein the monoclonal antibody or antigen-binding portion possesses one or more properties selected from the group consisting of:
(a) reduces endosialin binding to fibronectin;
(b) reduces fibronectin binding to endosialin with an IC₅₀ of 75 μg/mL or lower;
(c) reduces endosialin-mediated angiogenesis;
(d) reduces growth of an endosialin-expressing tumor;
(e) reduces proliferation of human epidermoid cancer cells; and
(f) reduces endosialin-mediated signalling.

4. The rabbit monoclonal antibody or antigen-binding portion of claim 1, which does not bind endosialin in the lectin domain.

5. The rabbit monoclonal antibody or antigen binding portion of claim 1, which is useful for immunohistochemistry (IHC) staining.

6. The rabbit monoclonal antibody or antigen-binding portion of claim 5, wherein the IHC staining is in a frozen tissue sample or in a formalin-fixed, paraffin-embedded tissue sample.

7. The rabbit monoclonal antibody of claim 6 which stains a formalin-fixed, paraffin-embedded tissue sample at least twice the visual intensity of non-specific, background control staining.

8. The rabbit monoclonal antibody or antigen-binding portion according to claim 2, wherein said antibody or antigen-binding portion is selected from the group consisting of:
(a) an antibody or antigen-binding portion that internalizes in endosialin-expressing cells;
(b) an antibody or antigen-binding portion that exhibits antibody dependent cellular cytotoxicity (ADCC);
(c) an antibody or antigen-binding portion that exhibits complement-dependent cytotoxicity (CDC);
(d) an antibody or antigen-binding portion that stains a formalin-fixed, paraffin embedded tissue sample with substantially the same intensity as humanized antibody 1-55-2, humanized antibody 1-3-1, humanized antibody 1-25-2, antibody 1-55-2, antibody 1-25-2 or antibody 1-3-1; and
(e) any combination of (a)-(d)

9. A monoclonal antibody or an antigen-binding portion thereof that specifically binds endosialin, wherein the antibody is selected from the group consisting of:
(a) an antibody comprising a heavy chain CDR3 sequence comprising SEQ ID NO: 238, 20, 52, 84, 116, 163 or 206;
(b) an antibody comprising a light chain CDR3 sequence comprising SEQ ID NO: 254, 36, 68, 100, 132, 181 or 222;
(c) an antibody comprising a heavy chain CDR3 sequence comprising SEQ ID NO: 238 and a light chain CDR 3 sequence comprising SEQ ID NO: 254
(d) an antibody comprising a heavy chain CDR3 sequence comprising SEQ ID NO: 20 and a light chain CDR3 sequence comprising SEQ ID NO: 36;
(e) an antibody comprising a heavy chain CDR3 sequence comprising SEQ ID NO: 52 and a light chain CDR 3 sequence comprising SEQ ID NO: 68;
(f) an antibody comprising a heavy chain CDR3 sequence comprising SEQ ID NO: 84 and a light chain CDR 3 sequence comprising SEQ ID NO: 100;
(g) an antibody comprising a heavy chain CDR3 sequence comprising SEQ ID NO: 116 and a light chain CDR 3 sequence comprising SEQ ID NO: 132;
(h) an antibody comprising a heavy chain CDR3 sequence comprising SEQ ID NO: 163 and a light chain CDR 3 sequence comprising SEQ ID NO: 181; and
(i) an antibody comprising a heavy chain CDR3 sequence comprising SEQ ID NO: 206 and a light chain CDR 3 sequence comprising SEQ ID NO: 222.

10. The antibody or antigen-binding portion according to claim 9, wherein the antibody is selected from the group consisting of:
(a) an antibody that comprises heavy chain CDR1, CDR2 and CDR3 regions comprising SEQ ID NOS: 234, 236 and 238, respectively;
(b) an antibody that comprises heavy chain CDR1, CDR2 and CDR3 regions comprising SEQ ID NOS: 16, 18 and 20, respectively;
(c) an antibody that comprises heavy chain CDR1, CDR2 and CDR3 regions comprising SEQ ID NOS: 48, 50 and 52, respectively;
(d) an antibody that comprises heavy chain CDR1, CDR2 and CDR3 regions comprising SEQ ID NOS: 80, 82 and 84, respectively;
(e) an antibody that comprises heavy chain CDR1, CDR2 and CDR3 regions comprising SEQ ID NOS: 112, 114 and 116, respectively;
(f) an antibody that comprises heavy chain CDR1, CDR2 and CDR3 regions comprising SEQ ID NOS: 159, 161 and 163, respectively;
(g) an antibody that comprises heavy chain CDR1, CDR2 and CDR3 regions comprising SEQ ID NOS: 202, 204 and 206, respectively;
(h) an antibody that comprises light chain CDR1, CDR2 and CDR3 regions comprising SEQ ID NOS: 250, 252, and 254, respectively;
(i) an antibody that comprises light chain CDR1, CDR2 and CDR3 regions comprising SEQ ID NOS: 32, 34 and 36, respectively;
(j) an antibody that comprises light chain CDR1, CDR2 and CDR3 regions comprising SEQ ID NOS: 64, 66 and 68, respectively;
(k) an antibody that comprises light chain CDR1, CDR2 and CDR3 regions comprising SEQ ID NOS: 96, 98 and 100, respectively;
(l) an antibody that comprises light chain CDR1, CDR2 and CDR3 regions comprising SEQ ID NOS: 128, 130 and 132, respectively;
(m) an antibody that comprises light chain CDR1, CDR2 and CDR3 regions comprising SEQ ID NOS: 177, 179, and 181, respectively;
(n) an antibody that comprises light chain CDR1, CDR2 and CDR3 regions comprising SEQ ID NOS: 218, 220, and 222, respectively;
(o) an antibody that comprises heavy chain CDR1, CDR2 and CDR3 regions comprising SEQ ID NOS: 234, 236 and 238, respectively, and comprises light chain CDR1, CDR2 and CDR3 regions comprising SEQ ID NOS: 250, 252 and 254, respectively;
(p) an antibody that comprises heavy chain CDR1, CDR2 and CDR3 regions comprising SEQ ID NOS: 16, 18 and 20, respectively, and comprises light chain CDR1, CDR2 and CDR3 regions comprising SEQ ID NOS: 32, 34 and 36, respectively;
(q) an antibody that comprises heavy chain CDR1, CDR2 and CDR3 regions comprising SEQ ID NOS: 48, 50 and 52, respectively, and comprises light chain CDR1, CDR2 and CDR3 regions comprising SEQ ID NOS: 64, 66 and 68, respectively;
(r) an antibody that comprises heavy chain CDR1, CDR2 and CDR3 regions comprising SEQ ID NOS: 80, 82 and 84, respectively, and comprises light chain CDR1, CDR2 and CDR3 regions comprising SEQ ID NOS: 96, 98 and 100, respectively;
(s) an antibody that comprises heavy chain CDR1, CDR2 and CDR3 regions comprising SEQ ID NOS: 112, 114 and 116, respectively, and comprises light chain CDR1, CDR2 and CDR3 regions comprising SEQ ID NOS: 128, 130 and 132, respectively;
(t) an antibody that comprises heavy chain CDR1, CDR2 and CDR3 regions comprising SEQ ID NOS: 159, 161 and 163, respectively, and comprises light chain CDR1, CDR2 and CDR3 regions comprising SEQ ID NOS: 177, 179 and 181, respectively; and
(u) an antibody that comprises heavy chain CDR1, CDR2 and CDR3 regions comprising SEQ ID NOS: 202, 204 and 206, respectively, and comprises light chain CDR1, CDR2 and CDR3 regions comprising SEQ ID NOS: 218, 220 and 222, respectively.

11. A monoclonal antibody or an antigen-binding portion thereof that specifically binds endosialin, wherein the antibody is selected from the group consisting of:
(a) an antibody comprising a heavy chain variable region comprising an amino acid sequence that is at least 85% identical to the sequence of SEQ ID NO: 232, 14, 46, 78, 110, 157 or 200;
(b) an antibody comprising a light chain variable region comprising an amino acid sequence that is at least 85% identical to the sequence of SEQ ID NO: 248, 30, 62, 94, 126, 175 or 216;
(c) an antibody comprising a heavy chain variable region comprising an amino acid sequence that is at least 85% identical to the sequence of SEQ ID NO: 232 and a light chain variable region comprising an amino acid sequence that is at least 85% identical to the sequence of SEQ ID NO: 248;
(d) an antibody comprising a heavy chain variable region comprising an amino acid sequence that is at least 85% identical to the sequence of SEQ ID NO: 14 and a light chain variable region comprising an amino acid sequence that is at least 85% identical to the sequence of SEQ ID NO: 30;
(e) an antibody comprising a heavy chain variable region comprising an amino acid sequence that is at least 85% identical to the sequence of SEQ ID NO: 46 and a light chain variable region comprising an amino acid sequence that is at least 85% identical to the sequence of SEQ ID NO: 62;
(f) an antibody comprising a heavy chain variable region comprising an amino acid sequence that is at least 85% identical to the sequence of SEQ ID NO: 78 and a light chain variable region comprising an amino acid sequence that is at least 85% identical to the sequence of SEQ ID NO: 94;
(g) an antibody comprising a heavy chain variable region comprising an amino acid sequence that is at least 85% identical to the sequence of SEQ ID NO: 110 and a light chain variable region comprising an amino acid sequence that is at least 85% identical to the sequence of SEQ ID NO: 126;
(h) an antibody comprising a heavy chain variable region comprising an amino acid sequence that is at least 85% identical to the sequence of SEQ ID NO: 157 and a light chain variable region comprising an amino acid sequence that is at least 85% identical to the sequence of SEQ ID NO: 175; and
(i) an antibody comprising a heavy chain variable region comprising an amino acid sequence that is at least 85% identical to the sequence of SEQ ID NO: 200 and a light chain variable region comprising an amino acid sequence that is at least 85% identical to the sequence of SEQ ID NO: 216.

12. The antibody or antigen-binding portion according to claim 11, wherein the antibody is selected from the group consisting of:
(a) an antibody comprising a heavy chain variable region comprising an amino acid sequence that is at least 95% identical to the sequence of SEQ ID NO: 232, 14, 46, 78, 110, 157 or 200;
(b) an antibody comprising a light chain variable region comprising an amino acid sequence that is at least 95% identical to the sequence of SEQ ID NO: 248, 30, 62, 94, 126, 175 or 216;
(c) an antibody comprising a heavy chain variable region comprising an amino acid sequence that is at least 95% identical to the sequence of SEQ ID NO: 232 and a light chain variable region comprising an amino acid sequence that is at least 95% identical to the sequence of SEQ ID NO: 248;
(d) an antibody comprising a heavy chain variable region comprising an amino acid sequence that is at least 95% identical to the sequence of SEQ ID NO: 14 and a light chain variable region comprising an amino acid sequence that is at least 95% identical to the sequence of SEQ ID NO: 30;
(e) an antibody comprising a heavy chain variable region comprising an amino acid sequence that is at least 95% identical to the sequence of SEQ ID NO: 46 and a light chain variable region comprising an amino acid sequence that is at least 95% identical to the sequence of SEQ ID NO: 62;
(f) an antibody comprising a heavy chain variable region comprising an amino acid sequence that is at least 95% identical to the sequence of SEQ ID NO: 78 and a light chain variable region comprising an amino acid sequence that is at least 95% identical to the sequence of SEQ ID NO: 94;
(g) an antibody comprising a heavy chain variable region comprising an amino acid sequence that is at least 95% identical to the sequence of SEQ ID NO: 110 and a light chain variable region comprising an amino acid sequence that is at least 95% identical to the sequence of SEQ ID NO: 126;
(h) an antibody comprising a heavy chain variable region comprising an amino acid sequence that is at least 95% identical to the sequence of SEQ ID NO: 157 and a light chain variable region comprising an amino acid sequence that is at least 95% identical to the sequence of SEQ ID NO: 175; and
(i) an antibody comprising a heavy chain variable region comprising an amino acid sequence that is at least 95% identical to the sequence of SEQ ID NO: 200 and a light chain variable region comprising an amino acid sequence that is at least 95% identical to the sequence of SEQ ID NO: 216.

13. The antibody or antigen-binding portion according to claim 11, wherein the antibody is selected from the group consisting of:
(a) an antibody comprising a heavy chain variable region comprising the sequence of SEQ ID NO: 232, 14, 46, 78, 110, 157 or 200;
(b) an antibody comprising a light chain variable region comprising the sequence of SEQ ID NO: 248, 30, 62, 94, 126, 175 or 216;
(c) an antibody comprising a heavy chain variable region comprising the sequence of SEQ ID NO: 232 and a light chain variable region comprising the sequence of SEQ ID NO: 248;
(d) an antibody comprising a heavy chain variable region comprising the sequence of SEQ ID NO: 14 and a light chain variable region comprising the sequence of SEQ ID NO: 30;
(e) an antibody comprising a heavy chain variable region comprising the sequence of SEQ ID NO: 46 and a light chain variable region comprising the sequence of SEQ ID NO: 62;
(f) an antibody comprising a heavy chain variable region comprising the sequence of SEQ ID NO: 78 and a light chain variable region comprising the sequence of SEQ ID NO: 94;
(g) an antibody comprising a heavy chain variable region comprising the sequence of SEQ ID NO: 110 and a light chain variable region comprising the sequence of SEQ ID NO: 126;
(h) an antibody comprising a heavy chain variable region comprising the sequence of SEQ ID NO: 157 and a light chain variable region comprising the sequence of SEQ ID NO: 175; and
(i) an antibody comprising a heavy chain variable region comprising the sequence of SEQ ID NO: 200 and a light chain variable region comprising the sequence of SEQ ID NO: 216.

14. The antibody of claim 13, which is a humanized antibody or an antigen-binding portion of said humanized antibody.

15. A monoclonal antibody that specifically binds endosialin, wherein the antibody comprises heavy chain CDR1, CDR2 and CDR3 regions comprising SEQ ID NOS: 234, 236 and 238, respectively, and comprises light chain CDR1, CDR2 and CDR3 regions comprising SEQ ID NOS: 250, 252 and 254, respectively.

16. The antibody according to claim 15, wherein the antibody comprises a heavy chain variable region comprising the sequence of SEQ ID NO: 232 and a light chain variable region comprising the sequence of SEQ ID NO: 248.

17. The antibody according to claim 15, wherein the antibody comprises a heavy chain sequence of SEQ ID NO: 189, with or without a signal sequence, and a light chain sequence of SEQ ID NO: 191, with or without a signal sequence.

18. The humanized antibody according to claim 14 comprising a human immunoglobulin heavy chain constant region, wherein said constant region is selected from the group consisting of: a human IgM constant region, a human IgG1 constant region, a human IgG2 constant region, a human IgG3 constant region, a human IgG4 constant region, a human IgE constant region A1 and a human IgA2M constant region.

19. A hybridoma cell line selected from the group consisting of: 1-55-2 (ATCC Deposit No. PTA-9652), 1-3-1 (ATCC Deposit No. PTA-9653), 1-25-2 (ATCC Deposit No. PTA-9650) and Clone 8 (ATCC Deposit No. PTA-9651).

20. A monoclonal antibody comprising the heavy chain amino acid sequence, light chain amino acid sequence or both, of the monoclonal antibody produced by a hybridoma cell line of claim 19.

21. The antibody according to claim 9 or an antigen-binding portion thereof that is detectably labeled.

22. The antibody according to claim 21, wherein the antibody is labeled with fluorescein, fluorescein isothiocyanate, rhodamine, phycoerythrin, 5-dimethylamine-1-napthalenesulfonyl chloride, lanthanide phosphor, horseradish peroxidase, β-galactosidase, luciferase, alkaline phosphatase, glucose oxidase, biotin, a radiolabeled amino acid, a paramagnetic ion, a radioactive ion, a fluorogenic ion or an X-ray imaging agent.

23. A composition comprising the antibody or antigen-binding portion according to claim 9.

24. The composition according to claim 23, further comprising an agent selected from a diagnostic agent and a therapeutic agent.

25. An immunohistochemistry assay kit comprising an antibody or antigen-binding portion according to claim 9.

26. An isolated nucleic acid molecule selected from the group consisting of:
(a) a nucleic acid molecule comprising SEQ ID NO: 224;
(b) a nucleic acid molecule comprising SEQ ID NO: 188, with or without a signal sequence;
(c) a nucleic acid molecule comprising SEQ ID NO: 6;
(d) a nucleic acid molecule comprising SEQ ID NO: 38;
(e) a nucleic acid molecule comprising SEQ ID NO: 70;
(f) a nucleic acid molecule comprising SEQ ID NO: 102;
(g) a nucleic acid molecule comprising SEQ ID NO: 148;
(h) a nucleic acid molecule comprising SEQ ID NO: 147, with or without a signal sequence;
(i) a nucleic acid molecule comprising SEQ ID NO: 192;
(j) a nucleic acid molecule comprising SEQ ID NO: 184, with or without a signal sequence;
(k) a nucleic acid molecule comprising SEQ ID NO: 240;
(l) a nucleic acid molecule comprising SEQ ID NO: 190, with or without a signal sequence;
(m) a nucleic acid molecule comprising SEQ ID NO: 22;
(n) a nucleic acid molecule comprising SEQ ID NO: 54;
(o) a nucleic acid molecule comprising SEQ ID NO: 86;
(p) a nucleic acid molecule comprising SEQ ID NO: 118;
(q) a nucleic acid molecule comprising SEQ ID NO: 166;
(r) a nucleic acid molecule comprising SEQ ID NO: 165, with or without a signal sequence;
(s) a nucleic acid molecule comprising SEQ ID NO: 208;
(t) a nucleic acid molecule comprising SEQ ID NO: 186, with or without a signal sequence;
(u) a nucleic acid molecule comprising SEQ ID NO: 224 and SEQ ID NO: 240;
(v) a nucleic acid molecule comprising SEQ ID NO: 188, with or without a signal sequence, and SEQ ID NO: 190, with or without a signal sequence;
(w) a nucleic acid molecule comprising SEQ ID NO: 6 and SEQ ID NO: 22;
(x) a nucleic acid molecule comprising SEQ ID NO: 38 and SEQ ID NO: 54;
(y) a nucleic acid molecule comprising SEQ ID NO: 70 and SEQ ID NO: 86;
(z) a nucleic acid molecule comprising SEQ ID NO: 102 and SEQ ID NO: 118;
(aa) a nucleic acid molecule comprising SEQ ID NO: 148 and SEQ ID NO: 166;
(bb) a nucleic acid molecule comprising SEQ ID NO: 192 and SEQ ID NO: 208;
(cc) a nucleic acid molecule comprising SEQ ID NO: 147, with or without a signal sequence, and SEQ ID NO: 165, with or without a signal sequence; and
(dd) a nucleic acid molecule comprising SEQ ID NO: 184, with or without a signal sequence, and SEQ ID NO: 186, with or without a signal sequence.

27. A vector comprising a nucleic acid molecule according to claim 26.

28. A host cell comprising a nucleic acid molecule according to claim 26.

29. A host cell comprising a vector according to claim 27.

30. A method for producing an antibody that specifically binds endosialin or an antigen-binding portion of said antibody, comprising culturing a host cell according to claim 28 under suitable conditions and recovering said antibody or antigen-binding portion.

31. A method for producing an antibody that specifically binds endosialin or an antigen-binding portion of said antibody, comprising culturing a host cell according to claim 29 under suitable conditions and recovering said antibody or antigen-binding portion.

32. A method for detecting endosialin in a cell or tissue comprising the steps of:
(a) contacting the cell or tissue with an anti-endosialin antibody or an antigen-binding portion thereof according to claim 9; and
(b) detecting association of said antibody or antigen-binding portion with said cell or tissue.

33. A method for identifying a candidate for treatment that targets endosialin-bearing cells comprising obtaining information on the presence or absence of endosialin in a sample from a patient, wherein the presence of endosialin in the sample is indicated by binding of an antibody or antigen-binding portion according to claim 9 and wherein the presence of endosialin in the sample indicates that the patient is a candidate for said treatment.

34. A method for reducing endosialin binding to fibronectin comprising the step of exposing the endosialin to an antibody or antigen-binding portion according to claim 9 under conditions that permit complexing of said antibody or antigen-binding portion with the endosialin.

35. A method for reducing endosialin-mediated angiogenesis in a subject in need thereof, comprising the step of administering an antibody or antigen-binding portion according to claim 9.

36. A method for reducing the growth of an endosialin-expressing tumor in a subject comprising administering an antibody or antigen-binding portion according to claim 9.

37. A method for monitoring treatment of a subject in need thereof with an endosialin antagonist comprising the steps of:
(a) obtaining a biological sample containing cells or tissue from the subject after treatment with the endosialin antagonist; and
(b) detecting and/or measuring endosialin in the sample using an antibody or antigen-binding portion according to claim 9;
wherein reduced endosialin compared to the amount in a previous sample indicates that the antagonist is effective.
